SEQUENCE LISTING (4 sequences)

SEQ ID NO: 1 - RNA, 31 nt, Artificial sequence
  Synthetic aptamer
guggacuauaccgcguaaugcugccuccacn

SEQ ID NO: 2 - RNA, 15 nt, Artificial Sequence
  Synthetic oligonucleotide
cgcgguauaguccac

SEQ ID NO: 3 - RNA, 29 nt, Artificial Sequence
  Synthetic oligonucleotide
ggaggacggcgcgccuggcauaagccucc

SEQ ID NO: 4 - RNA, 12 nt, Artificial sequence
  Synthetic aptamer
uuaugccaggcg

(19) United States
(12) Patent Application Publication
(10) Pub. No.: US 2014/0309287 A1
(43) Pub. Date: Oct. 16, 2014

(54) OLIGONUCLEOTIDE FORMULATION

(71) Applicants: CHRISTOPHER P. RUSCONI, Durham, NC (US); DOUGLAS BROOKS, Durham, NC (US); ALISON G. FLOYD, Raleigh, NC (US)

(72) Inventors: CHRISTOPHER P. RUSCONI, Durham, NC (US); DOUGLAS BROOKS, Durham, NC (US); ALISON G. FLOYD, Raleigh, NC (US)

(73) Assignee: REGADO BIOSCIENCES, INC., Basking Ridge, NJ (US)

(21) Appl. No.: 14/099,586

(22) Filed: Dec. 6, 2013

Related U.S. Application Data
(60) Provisional application No. 61/734,277, filed on Dec. 6, 2012.
(60) Provisional application No. 61/798,466, filed on Mar. 15, 2013.

(51) Int. Cl. A61K 47/20; A61K 31/7088; A61K 47/48

(52) U.S. Cl. CPC A61K 47/20; A61K 47/48215; A61K 31/7088
USPC 514/44 R

(57) ABSTRACT

The present invention relates generally to pharmaceutical formulations of conjugated oligonucleotides, such as pegylated aptamers, as well as to a method of preparing and using the same. The pharmaceutical formulations have desirable shelf life characteristics under varied storage conditions. Formulations of oligonucleotides or oligonucleotides conjugates and methods for their preparation and use are provided having a shelf life of at least about 24 months or, more preferably, at least about 36 months at about 2 to about 30° C. In particular embodiments, the formulations have a pH of about 7 or less and contain methionine. Optionally, dissolved oxygen content is reduced during the manufacturing process is used to improve desired shelf life characteristics.

### CROSS REFERENCE TO RELATED APPLICATIONS

This application claims the benefit of U.S. provisional patent application 61/734,277 filed Dec. 6, 2012 and U.S. provisional patent application 61/798,466 filed Mar. 15, 2013, the contents of which are hereby incorporated by reference in their entirety.

### FIELD OF INVENTION

The present invention relates generally to pharmaceutical formulations of conjugated oligonucleotides, such as pegylated aptamers, as well as to a method of preparing and using the same. The formulations have desirable shelf life characteristics under varied conditions.

### BACKGROUND OF THE INVENTION

Oligonucleotide therapeutics represent a promising class of therapeutic agents distinguished by their structure, target and mechanism of action. Numerous agents within the class are currently in development, including aptamers, antisense molecules, ribozymes and interfering RNAs.

The therapeutic profile of agents in this class is often improved by conjugation of the oligonucleotide of interest to another molecule. Conjugates include, for example, peptides and proteins, carbohydrates, antibodies, enzymes, polymers, drugs and fluorophores. Conjugation to polyethylene glycol (PEG), for example, can improve the pharmacokinetic, pharmacodynamic, and immunological profile of an oligonucleotide therapeutic, and thus, enhance its therapeutic effect. A branched-PEG anti-VEGF aptamer, pegaptanib (Macugen®), was approved by FDA in 2004 for the treatment of age-related macular degeneration.

Despite its benefits, conjugation to PEG (known as pegylation) can have an adverse effect on the shelf life or potency of an oligonucleotide therapeutic because PEG is susceptible to auto-oxidation. Knop, K. et al. Chem. Int. Ed., 2010, 49, 6288-6308; Han, S. et al., Polymer Vol. 38 No. 2, pp. 317-33, 1997. Oxidation can be initiated by heat, light, or transition metals and is propagated by oxygen or other oxidizers. As oxidation progresses, PEG degrades into a family of related substances with different molecular weights, which can be observed chromatographically. Yang et al. Eur. Polym. J. 32 (1996), 535-547; Han et al. Polymer, 38 (1997), 317-323.

There remains a need to improve the shelf life of conjugated oligonucleotide therapeutics, including pegylated oligonucleotides.

### SUMMARY OF INVENTION

The present invention relates to pharmaceutical formulations of conjugated oligonucleotides, such as pegylated aptamers, as well as to methods of preparing and using the same. The pharmaceutical formulations have a shelf life of at least about twenty four (24) months, or more preferably, at least about thirty six (36) months under varied storage conditions.

In one aspect, the present invention is a pharmaceutical formulation comprising a pegylated aptamer and methionine, wherein the formulation has a pH of about 7 or less, an ambient or reduced dissolved oxygen level and a shelf life of at least about 24 months, or, more preferably, at least about thirty six (36) months under varied storage conditions.

The concentration of the pegylated aptamer may vary. In a particular embodiment, the concentration of the pegylated aptamer is from about 1 to about 100, about 20 to about 30, or about 24 mg/mL.

The concentration of methionine may vary. In a particular embodiment, methionine is present from about 0.001 to about 0.50% w/w, about 0.10 to about 0.25 or about 0.10% w/w.

The level of dissolved oxygen in the pharmaceutical formulation may vary. In a particular embodiment, the level of dissolved oxygen is ambient or between about 5 and about 10 ppm. In another particular embodiment, the level of dissolved oxygen is reduced or less than about 5 ppm.

The pharmaceutical formulations of the present invention advantageously possess a shelf-life of at least about 24 or, more preferably, at least about 36 months, under varied storage conditions. The temperature conditions to which the formulation is subjected may vary. In a particular embodiment, the temperature is between about 2° C. and about 8° C. In another particular embodiment, the temperature is between about 25° C. and about 30° C. In a specific embodiment, the temperature is about 25° C.

In an exemplary embodiment, the present invention is a pharmaceutical formulation comprising from about 20 to about 30 mg/mL of pegnivacogin and from about 0.025 to about 0.5% w/w methionine, wherein the formulation has a pH of about 7 or less, an ambient or reduced dissolved oxygen level and a shelf life of at least about 24 months, or, more preferably, at least about thirty six (36) months under varied storage conditions.

In another exemplary embodiment, the present invention is a pharmaceutical formulation comprising about 24 mg/mL of pegnivacogin and about 0.10% w/w methionine, wherein the formulation has a pH of about 7 or less, or more particularly about 6.8, an ambient or reduced dissolved oxygen level and a shelf life of at least about 24 months, or, more preferably, at least about thirty six (36) months, under varied storage conditions.

In yet another exemplary embodiment, the present invention is a pharmaceutical formulation comprising about 24 mg/mL of pegnivacogin and about 0.10% w/w methionine, wherein the formulation has a pH of about 6.8, a dissolved oxygen level of between about 5 and about 10 ppm and a shelf life of at least about 24 months, or more preferably at least about thirty six (36) months at a temperature of about 2° C. to about 8° C. or about 25° C. to about 30° C.

In a second aspect, the present invention is a method for preparing a pharmaceutical formulation. In one embodiment, the method comprises: (a) providing an aqueous solvent comprising methionine; (b) adding a pegylated aptamer to the aqueous solvent to form an aqueous formulation; (c) filtering the aqueous formulation to form a filtered aqueous formulation; (d) filling a storage container with the filtered aqueous formulation; and (e) sealing the storage container to provide a pharmaceutical formulation having a pH of about 7 or less, an ambient or reduced dissolved oxygen level and a shelf life of at least about 24 months, or, more preferably, at least about thirty six (36) months under varied storage conditions.

The pegylated aptamer may be added to the aqueous solvent as a solid or an aqueous solution, in the latter instance where the pegylated aptamer has been previously dissolved in a dissolving solvent.

In another embodiment, the method comprises: (a) providing an aqueous solvent; (b) adding a pegylated aptamer and methionine to the aqueous solvent to form an aqueous formulation; (c) filtering the aqueous formulation to form a filtered aqueous formulation; (d) filling a storage container with the filtered aqueous formulation; and (e) sealing the storage container to provide a pharmaceutical formulation having a pH of about 7 or less, an ambient or reduced dissolved oxygen level and a shelf life of at least about 24 months, or, more preferably, at least about thirty six (36) months under varied storage conditions. According to this embodiment of the method, the timing of the addition of methionine to the aqueous solvent may vary. In one embodiment, methionine is added to the aqueous solvent simultaneously with the pegylated aptamer. In another embodiment, methionine is added to the aqueous formulation after the pegylated aptamer.

Optionally, the method may further comprise one or more additional steps. In one embodiment, the method further comprises modifying the pH of one or more of the solvent, the dissolving solvent, the aqueous formulation, the filtered aqueous formulation or combinations thereof, one or more times. In particular embodiments, the pH may be measured prior to and/or after a pH modification step.

In another embodiment, the method further comprises modifying the dissolved oxygen level of one or more of the solvent, the aqueous formulation, the filtered aqueous formulation or combinations thereof, one or more times. The dissolved oxygen level can be reduced, for example, by sparging with nitrogen or other acceptable medical gas. In particular embodiments, the dissolved oxygen level may be measured prior to and/or after a dissolved oxygen modification step.

In an exemplary embodiment, the method comprises (a) providing an aqueous solvent comprising methionine; (b) adding a pegylated aptamer to the aqueous solvent to form an aqueous formulation; (c) filtering the aqueous formulation to form a filtered aqueous formulation; (d) filling a storage container with the filtered aqueous formulation; and (e) sealing the storage container to provide a pharmaceutical formulation having a pH of about 7 or less, an ambient dissolved oxygen level, and a shelf life of at least about 24 months, or more preferably, at least about thirty six (36) months under varied storage conditions included, but not limited to, temperatures between about 2° C. and about 8° C. or about 25° C. and about 30° C.

In another exemplary embodiment, the method comprises (a) providing an aqueous solvent; (b) adding a pegylated aptamer and methionine to the aqueous solvent to form an aqueous formulation; (c) filtering the aqueous formulation to form a filtered aqueous formulation; (d) filling a storage container with the filtered aqueous formulation; and (e) sealing the storage container to provide a pharmaceutical formulation having a pH of about 7 or less, a dissolved oxygen level of about 5 to about 10 ppm, and a shelf life of at least about 24 months, or more preferably, at least about thirty six (36) months under varied storage conditions included, but not limited to, temperatures between about 2° C. and about 8° C. or about 25° C. and about 30° C.

In a preferred embodiment, the present invention is a method of preparing a pharmaceutical formulation, comprising (a) providing an aqueous solvent comprising methionine; (b) adding pegnivacogin to the aqueous solvent to form an aqueous formulation; (c) filtering the aqueous formulation to form a filtered aqueous formulation; (d) filling a storage container with the filtered aqueous formulation; and (e) sealing the storage container to provide a pharmaceutical formulation having a pH of about 7 or less, an ambient dissolved oxygen level, and a shelf life of at least about 24 months, or more preferably, at least about thirty six (36) months under varied storage conditions included, but not limited to, temperatures between about 2° C. and about 8° C. or about 25° C. and about 30° C.

In another preferred embodiment, the method comprises (a) providing an aqueous solvent; (b) adding pegnivacogin and methionine to the aqueous solvent to form an aqueous formulation; (c) filtering the aqueous formulation to form a filtered aqueous formulation; (d) filling a storage container with the filtered aqueous formulation; and (e) sealing the storage container to provide a pharmaceutical formulation having a pH of about 7 or less, a dissolved oxygen level of about 5 to about 10 ppm, and a shelf life of at least about 24 months, or more preferably, at least about thirty six (36) months under varied storage conditions included, but not limited to, temperatures between about 2° C. and about 8° C. or about 25° C. and about 30° C.

In a third aspect, the present invention is a method of treating a host in need thereof by administering a therapeutically effective amount of the pharmaceutical formulation of the present invention.

In a particular embodiment, the host is in need of anticoagulation. In a specific embodiment, the pharmaceutical formulation comprises pegnivacogin and the host is undergoing a coronary revascularization procedure such as CABG or PCI.

In another particular embodiment, the host is suffering from a platelet mediated disorder. In a specific embodiment, the pharmaceutical formulation comprises RB571 and the host is suffering from ACS, rheumatoid arthritis or diabetic vasculopathy.

In another particular embodiment, the host is suffering from structural heart disease such as valvular heart disease. In a specific embodiment, the host is undergoing a transcatheter aortic valve replacement or implantation (TAVR/TAVI).

In one embodiment, the pharmaceutical formulation is administered intravenously. In a preferred embodiment, the formulation is administered as a bolus intravenous injection.

### BRIEF DESCRIPTION OF THE DRAWINGS

FIG. 1 presents overlays of AX-HPLC chromatograms for pegnivacogin formulations 040-100-R1-R17 stored for 12 months at 5° C. with nitrogen or air purging.

FIG. 2 presents overlays of AX-HPLC chromatograms for pegnivacogin formulations 040-100-R1-R17 stored for at least 24 months, 5° C. with nitrogen or air purging.

FIG. 3 presents overlays of AX-HPLC chromatograms of pegnivacogin injection (040-100R12), 21 mg/mL, pH 7.4, stored for 12 months at 5° C., 25° C. and 40° C.

FIG. 4 presents overlays of IP-HPLC chromatograms of pegnivacogin formulations 040-100-R1-R17 stored for 12 months at 5° C. with nitrogen and air purging.

FIG. 5 presents overlays of IP-HPLC chromatograms of pegnivacogin formulations 040-100-R1-R17 stored for 24 months at 5° C. with nitrogen and air purging.

FIG. 6 demonstrates the effects of temperature and pH effect on the stability of pegnivacogin formulations stored for 12 months via AX-HPLC

FIG. 7 demonstrates the effect of temperature and pH on the stability of pegnivacogin formulations stored for 36 months via AX-HPLC.

FIG. 8 demonstrates the effect of temperature and pH on the stability of pegnivacogin formulations containing antioxidants stored for 12 months via AX-HPLC.

FIG. 9 demonstrates the effect of temperature and pH on the stability of pegnivacogin formulations containing antioxidants stored for 36 months via AX-HPLC.

FIG. 10 demonstrates the effect of 0.1% Methionine and pH on Pegnivacogin Injection Stability at 40° C. (pH 6.5=R26, pH 7.0=R31, pH 7.4=R30, Phase I/II Fml=R25).

FIG. 11 demonstrates the effect of pH on the Impurity Profile of Pegnivacogin in Formulations Containing 0.1% w/w Methionine at 40° C. after 10½ Months (A) and 12 Months (B).

### DETAILED DESCRIPTION OF THE INVENTION

The present invention is directed to a pharmaceutical formulation of an oligonucleotide or conjugated oligonucleotide, such as a pegylated aptamer, as well as to methods of preparing and using the same. The pharmaceutical formulations of the present invention have a desirable shelf life under a variety of storage conditions. In a particular embodiment, the formulations have a shelf life of at least twenty four (24) months, or more preferably, at least about 36 months under varied storage conditions.

### I. Shelf Life

The shelf life of a pharmaceutical product is generally considered the time that the average drug characteristic (e.g., potency, sterility) remains within an approved specification after manufacture when stored according to specifications. Before a pharmaceutical product can be marketed, the manufacturer is generally required to apply for a label shelf life to the appropriate regulatory authority, e.g., the United States Food and Drug Administration (FDA) or the European Medicines Agency (EMEA). Since the true shelf-life of a drug product is usually unknown, it is typically estimated based on assay results of the drug characteristic from a stability study conducted during the process of drug development, as would be understood by one of skill in the art. Improved shelf life has significant economic advantages.

In one embodiment, shelf life refers to the period from the time of manufacture that the potency bioactivity of the oligonucleotide therapeutic agent such as the pegylated aptamer remains within the specifications when stored at recommended storage conditions.

In another embodiment, shelf life refers to the period of time from the time of manufacture that the bioactivity of the pegylated aptamer remains about 60%, about 70%, about 80%, about 90%, or 100% of bioactivity present at time of manufacture.

In another embodiment, the shelf life refers to the period of time from the time of manufacture that the bioactivity of the pegylated aptamer remains between about 50% to 150%, about 60% to 140%, about 70% to 130%, about 80%, to 120%, about 90% to 110%, about 95% to 105% of bioactivity present at time of manufacture.

In another embodiment, shelf life refers to the consistency of dosage units as described in terms of percent label claim. In one embodiment, shelf life refers to the period of time from the time of manufacture that the percent label claim remains between about 90% to about 110%, about 95% to about 105%, or about 98% to about 102% or about 99% to about 101%.

In another embodiment, shelf life refers to the period of time from the time of manufacture that the purity of the pegylated aptamer exceeds a specified level. In a preferred embodiment, the shelf life refers to the period of time from the time of manufacture that the percent purity exceeds about 90%, about 92%, about 94%, about 95%, about 99%, about 99.5%, or about 99.9%.

In another embodiment, the shelf life refers to the period of time from the time of manufacture that the content of total impurities remains below a specified level. In one embodiment, the shelf life refers to the period of time from the time of manufacture that the content of total impurities in the formulation is less than about 10%, less than about 8%, less than about 6%, less than about 5%, less than about 4%, less than about 3%, less than about 2%, less than about 1% or less than about 0.5%.

The content of different types of impurities in a formulation can be based on RRT (relative retention time) measurements. Impurities having a RRT greater than 1.01 (RRT≧1.01 impurities) represent oxidative degradants of PEG. In one embodiment, the shelf life refers to the period of time from the time of manufacture that the content of RRT≧1.01 impurities remains less than or equal to about 6%, less than about 5%, less than about 4%, less than about 3%, less than about 2%, or less than about 1%. Impurities having a RRT equal to 1.15 (RRT=1.15 impurities) represents cleavage of the linker between the arms of the branched PEG resulting in loss of one of the 20 kDa arms. While not to be bound by any specific mechanism, it is believed that generation of RRT=1.15 impurities is accelerated by oxidative processes (catalyzed by peroxy anions) and is also accelerated under basic conditions. In one embodiment, shelf life refers to the period of time from the time of manufacture that the content of RRT=1.15 impurities remains less than or equal to about 2%, less than about 1%, less than about 0.5% or less than about 0.1%.

The shelf life of the pharmaceutical formulation of the present invention is improved over known pharmaceutical formulations of conjugated oligonucleotide therapeutics, and in particular, the pharmaceutical formulation in Example 1.

In one embodiment, the shelf life is improved by about 5%, about 10%, about 15%, about 20%, about 25%, about 30%, about 35%, about 40%, about 45%, about 50% or greater than about 50% under the same storage conditions (e.g., temperature conditions).

In another embodiment, the shelf life of the pharmaceutical formulation of the present invention is about 1, about 2, about 3, about 4, about 5, about 6, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 38, about 40, about 42, about 44, about 46, about 48 or greater than about 48 months.

The improved shelf life of the pharmaceutical formulation of the present invention is permissible under varied storage conditions, including for example, temperature of storage. In one embodiment, the pharmaceutical formulation has an improved shelf life under refrigerated conditions. In a particular embodiment, the pharmaceutical formulation has an improved shelf life at a temperature of about 2° C. to about 8° C.

In another embodiment, the pharmaceutical formulation has an improved shelf life at a temperature of between about 8° C. and about 12° C., about 12° C. and about 18° C., about 18° C. and about 24° C.

In another embodiment, the pharmaceutical formulation has improved shelf life at room temperature, which permits room temperature handling, transport and storage. Room temperature refers to common indoor temperatures, which is commonly considered 25° C. in the United States and much of Europe. Room temperature may vary by region, however. In a particular embodiment, the pharmaceutical formulation has improved shelf life at about 25° C. to about 30° C., or about 25° C.

In another embodiment, the pharmaceutical formulation has improved shelf life at greater than about 30° C.

In one embodiment, the pharmaceutical formulation has a shelf life of at least about 12, about 14, about 16, about 18, about 20, about 22, about 24, about 26, about 28, about 30, about 32, about 34, about 36, about 38, about 40, about 42, about 44, about 46, about 48 or greater than about 48 months under refrigerated conditions.

In a particular embodiment, the pharmaceutical formulation has a shelf life of at least about 12, about 14, about 16, about 18, about 20, about 22, about 24, about 26, about 28, about 30, about 32, about 34, about 36, about 38, about 40, about 42, about 44, about 46, about 48 or greater than about 48 months between about 1 and about 15° C., about 1 and about 10° C. about 2 and about 8° C., or about 5° C.

In a particular embodiment, the pharmaceutical formulation has a shelf life of at least about 24 months between about 2° C. and about 8° C.

In another particular embodiment, the pharmaceutical formulation has a shelf life of at least about 36 months between about 2° C. and about 8° C.

In yet another particular embodiment, the pharmaceutical formulation has a shelf life of at least about 40 months between about 2° C. and about 8° C.

In yet another particular embodiment, the pharmaceutical formulation has a shelf life of at least about 42 months between about 2° C. and about 8° C.

In another particular embodiment, the pharmaceutical formulation has a shelf life of at least about 46 months between about 2° C. and about 8° C.

In another embodiment, the pharmaceutical formulation has a shelf life of at least about 12, about 14, about 16, about 18, about 20, about 22, about 24, about 26, about 28, about 30, about 32, about 34 or about 36 months at about 8° C. and about 24° C.

In a still further embodiment, the pharmaceutical formulation has a shelf life of at least about 12, about 14, about 16, about 18, about 20, about 22, about 24, about 26, about 28, about 30, about 32, about 34, about 36, about 38, about 40, about 42, about 44, about 46, about 48 months at room temperature. For example, the pharmaceutical formulation has a shelf life of at least about 24 months, or more preferably, at least about 36 months, at room temperature or between about 25° C. to 30° C. or about 25° C.

In a specific embodiment, the formulation permits a shelf life of from at least about 24 to at least about 48, at least about 26 to at least about 44, at least about 28 to at least about 42, at least about 30 to at least about 40, at least about 32 to at least about 38, or at least about 36 months under varied storage conditions, for example, between about 2° C. and about 8° C., about 8° C. and about 24° C., or about 25° C. to 30° C. and/or room temperature.

In another embodiment, the formulation has a shelf life of at least about 24 months, or more preferably, of at least about 36 months under refrigerated conditions. In a particular embodiment, the formulation has improved shelf life at about 2° C. to about 8° C.

In an exemplary embodiment, the shelf life of the formulation is improved by about 5, about 10, about 15, about 20, about 30, about 35, about 40, about 45, about 50, about 55, about 60, about 65, about 70, about 75, about 80, about 85, about 90, about 95 or about 100% or greater at about 2° C. to about 8° C.

In a specific embodiment, the formulation permits a shelf life of from at least about 24 to at least about 48, at least about 26 to at least about 44, at least about 28 to at least about 42, at least about 30 to at least about 40, at least about 32 to at least about 38, or at least about 36 months at 2° C. to about 8° C. and/or under refrigerated conditions.

In a still further embodiment, the formulation permits a shelf life of at least about 24 months, or more preferably, of at least about 36 months at greater than about 25° C.

In a particular embodiment, the formulation permits a shelf life of at least about 24 months, or more preferably, of at least about 36 months at greater than about 30° C.

In a particular embodiment, the formulation permits a shelf life of at least about 24 months, or more preferably, of at least about 36 months at greater than about 40° C.

### II. Oligonucleotides

The pharmaceutical formulation described herein is suitable for use with an oligonucleotide. Oligonucleotides are nucleic acid polymers typically fewer than about 50 nucleotides in length. They are known to be useful for research, diagnostic and therapeutic purposes.

In one embodiment, the oligonucleotide formulated according to the present invention is useful in research or diagnostics. That is, the term “pharmaceutical formulation” should not be construed to limit the use of the present invention to therapeutic methods.

In another embodiment, the oligonucleotide formulated according to the present invention is a therapeutic oligonucleotide, i.e., is useful in one or more methods of treatment of a disease or disorder in a subject in need thereof.

Oligonucleotide therapeutics are discriminated by their structure, function and mode of action. Structurally, they may be single or double-stranded DNA or RNA polymers ranging in size from about 10 to about 50 base pairs. The nucleic acid constituents and the oligonucleotide as a whole may be modified or unmodified, as described further herein. Their mechanism of action may vary. Generally, oligonucleotides exert their effect by binding to a target molecule, such as a nucleic acid sequence, protein or cell surface receptor, on the basis of complementary nucleic acid sequence or three dimensional configuration. Molecules within this class include, for example, aptamers, antisense molecules, ribozymes, deoxyribozymes, interfering RNA (siRNA, miRNA), decoys and immunostimulatory oligonucleotides. The oligonucleotides contemplated by the present invention can be synthesized by standard methods known in the art, e.g. by use of an automated DNA synthesizer (such as are commercially available from, for example, Biosearch, Applied Biosystems).

In one embodiment, the oligonucleotide suitable for use in the present invention is from about 10 to about 50 base pairs in length. In a particular embodiment, the oligonucleotide is from about 10 to about 20, about 20 to about 30, about 30 to about 40 or about 40 to about 50 base pairs in length.

In another embodiment, the oligonucleotide suitable for use in the present invention is selected from the group consisting of single-stranded RNA, double-stranded RNA, single-stranded DNA and double-stranded DNA oligonucleotides.

The oligonucleotide suitable for use in the present invention may be unmodified or comprise one or more modifications. These modifications may include, for example, modifications to the constituent nucleic acids or to the oligonucleotide molecule as a whole. Such modifications may be intended, for example, to increase the in vivo stability of the oligonucleotide or to enhance or to mediate delivery of the molecule.

In one embodiment, the oligonucleotide formulated according to the present invention comprises one or more modifications. Such modifications may provide, for example, additional charge, polarizability, hydrophobicity, hydrogen bonding, electrostatic interaction, and functionality to the nucleic acid or the oligonucleotide molecule as a whole.

In one embodiment, the oligonucleotide comprises one or more modifications to the base moiety, sugar moiety or phosphate backbone. Such modifications include, but are not limited to, 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, phosphorothioate or alkyl phosphate modifications, methylations, unusual base-pairing combinations such as the isobases isocytidine and isoguanidine and the like. Modifications can also include 3′ and 5′ modifications such as capping.

The oligonucleotide may also be modified by conjugation to a molecule or molecules having desired biological properties. Such molecules may include, without limitation, peptides and proteins, carbohydrates, antibodies, enzymes, polymers, drugs and fluorophores. In one embodiment, the oligonucleotide is conjugated to a lipophilic compound such as cholesterol, dialkyl glycerol, diacyl glycerol, or a non-immunogenic, high molecular weight compound or polymer such as polyethylene glycol (PEG) or other water soluble pharmaceutically acceptable polymers including, but not limited to, polyaminoamines (PAMAM) and polysaccharides such as dextran, or polyoxazolines (POZ).

PEG is formed by the additional reaction of ethylene oxide (EO) with mono ethylene glycols (MEG) or diethylene glycol. The generalized formula for PEG is: H(OCH₂ CH₂) n OH, wherein n=N: average number of repeating ethylene oxide groups. Compared to other polymers, PEG has a relatively narrow polydispersity (M/M) in the range of 1.0 for low molecular weight PEGs to 1.1 for high molecular weight PEGs. It is also soluble in both aqueous and organic solvents. PEG is rapidly cleared in vivo without structural change, where clearance is dependent upon molecular weight. It is only weakly immunogenic, even at high molecular weights. A general form of PEG that is used in pharmaceutical use is CH₃—(OCH₂CH₂)ₙ since methoxy PEG has improved stability as compared to —OH peg.

There are numerous sizes of PEG, represented by their average molecular weight. PEGs can range in size from about 5 to about 200 kD. Linear chain PEGs of up to about 30 kD can be produced. For PEGs of greater than 30 kD, multiple PEGs can be attached together (multi-arm or ‘branched’ PEGs) to produce PEGs of the desired size. The general synthesis of compounds with a branched, “mPEG2” attachment (two mPEGs linked via an amino acid) is described in Monfardini, et al., Bioconjugate Chem. 1995, 6:62-69. For ‘branched’ PEGs, i.e. compounds that include more than one PEG or mPEG linked to a common reactive group, the PEGs or mPEGs can be linked together through an amino acid such as a lysine or they can be linked via, for example, a glycerine. For branched PEGs in which each mPEG is about 10, about 20, or about 30 kD, the total mass is about 20, about 40 or about 60 kD and the compound is referred to by its total mass (i.e. 40 kD mPEG2 is two linked 20 kD mPEGs).

40 kD total molecular weight PEGs, that can be used as reagents in producing a pegylated compound, include, for example, [N²-(monomethoxy 20K polyethylene glycol carbamoyl)-N⁶-(monomethoxy 20K polyethylene glycol carbamoyl)]-lysine N-hydroxysuccinimide of the structure:

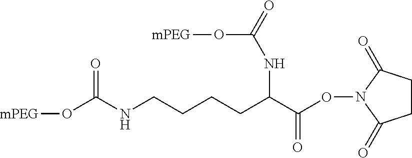

Additional PEG reagents that can be used to prepare stabilized compounds of the invention include other branched PEG N-Hydroxysuccinimide (mPEG-NHS) of the general formula:

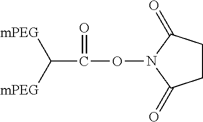

with a 40 kD or 60 kD total molecular weight (where each mPEG is about 20 or about 30 kD). As described above, the branched PEGs can be linked through any appropriate reagent, such as an amino acid, and in certain embodiments are linked via lysine residues or glycine residues.

They can also include non-branched mPEG-Succinimidyl Propionate (mPEG-SPA), of the general formula:

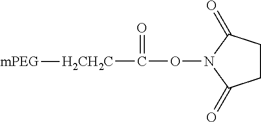

in which mPEG is about 20 kD or about 30 kD. In a specific embodiment, the reactive ester is —O—CH2CH2-CO2-NHS.

The reagents can also include a branched PEG linked through glycerol, such as the Sunbright™ series from NOF Corporation, Japan. Specific, non-limiting examples of these reagents are:

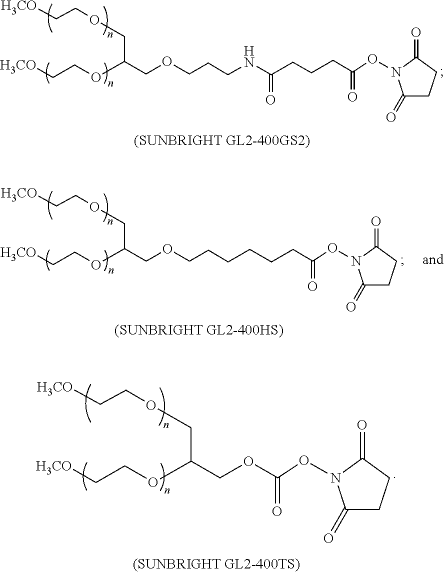

The reagents can also include non-branched Succinimidyl alpha-methylbutanoate (mPEG-SMB) of the general formula:

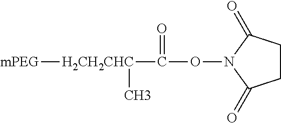

in which mPEG is between 10 and 30 kD. In a subembodiment, the reactive ester is —O—CH₂CH₂CH(CH.sub.3)-CO₂—NHS.

PEG reagents can also include nitrophenyl carbonate linked PEGs, such as of the following structure

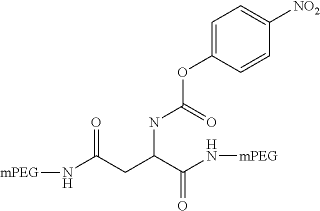

Compounds of this structure are commercially available, for example from Sunbio, Inc. Compounds including nitrophenyl carbonate can be conjugated to primary amine containing linkers. In this reaction, the O-nitrophenyl serves as the leaving group, leaving a structure [mPEG].sub.n-NH—CO—NH-linker-ligand.

PEGs with thiol-reactive groups that can be used with a thiol-modified linker, as described above, include compounds of the general structure:

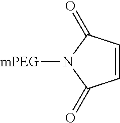

in which mPEG is about 10, about 20 or about 30 kD. Additionally, the structure can be branched, such as

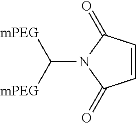

in which each mPEG is about 10, about 20, or about 30 kD and the total mass is about 20, about 40, or about 60 kD. Branched PEGs with thiol reactive groups that can be used with a thiol-modified linker, as described above, include compounds in which the branched PEG has a total molecular weight of about 40 or 60 kD (where each mPEG is 20 or 30 kD). PEG reagents can also be of the following structure:

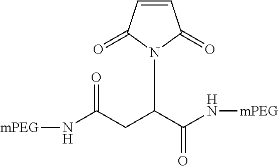

PEG-maleimide pegylates thiols of the target compound in which the double bond of the maleimic ring breaks to connect with the thiol. The rate of reaction is pH dependent and, in one embodiment, is carried out between pH 6 and 10, or between pH 7 and 9 or about pH 8.

In a particular embodiment, the oligonucleotide formulated according to the present invention is conjugated to PEG, i.e., a pegylated oligonucleotide. In a preferred embodiment, oligonucleotide is conjugated to PEG having a molecular weight from about 10 kD to about 60 kD, about 20 kD, about 30 kD, about 40 kD, about 50 kD or about 60 kD. In a particular embodiment, the oligonucleotide is conjugated to PEG having a molecular weight of about 40 kD.

In one embodiment, the oligonucleotide formulated according to the present invention is associated with a single PEG molecule. In another embodiment, the oligonucleotide is associated with two or more PEG molecules.

In yet a further embodiment, a plurality of PEG molecules can be attached to each other. In this embodiment, one or more oligonucleotides capable of binding to the same target or different targets can be associated with each PEG molecule. In embodiments where multiple oligonucleotides specific for the same target are attached to PEG, there is the possibility of bringing the same targets in close proximity to each other in order to generate specific interactions between the same targets. Where multiple oligonucleotides specific for different targets are attached to PEG, there is the possibility of bringing the distinct targets in close proximity to each other in order to generate specific interactions between the targets.

The molecule to be conjugated can be covalently bonded or associated through non-covalent interactions with the oligonucleotide of interest. In one embodiment, the molecule to be conjugated is covalently attached to the oligonucleotide. The covalent attachment may occur at a variety of positions on the oligonucleotide, e.g., to the exocyclic amino group on the base, the 5-position of a pyrimidine nucleotide, the 8-position of a purine nucleotide, the hydroxyl group of the phosphate, or a hydroxyl group or other group at the 5′ or 3′ terminus. In one embodiment, the covalent attachment is to the 5′ or 3′ hydroxyl group of the oligonucleotide.

Attachment of the lipophilic compound or non-immunogenic, high molecular weight compound molecule to other components of the complex can be direct or utilize linkers or spacers. Various linkers and attachment chemistries are known in the art. Several non-limiting embodiments are provided below. In one embodiment, an amino linker, such as the C 6 hexylamino linker, 6-(trifluoroacetamido)hexanol (2-cyanoethyl-N,N-diisopropyl)phosphoramidite, can be used to add the hexylamino linker to the 5′ end of the synthesized oligonucleotide. Other linker phosphoramidites that may be used to add linkers to the synthesized oligonucleotides are described below:

TFA-amino C4 CED phosphoramidite (available from ChemGenes, cat# CLP-1453) of the structure:

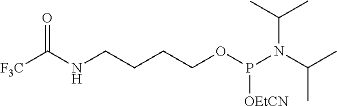

5′-amino modifier C3 TFA (available from Glen Research cat#10-1923-90) of the structure:

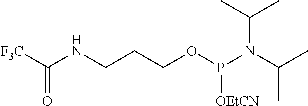

MMT amino modifier C6 CED phosphoramidite (available from Glen Research cat#10-1906)

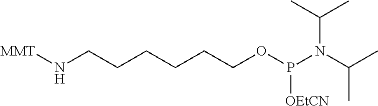

5′-amino modifier 5 (available from Glen Research cat#10-1905-90) of the structure:

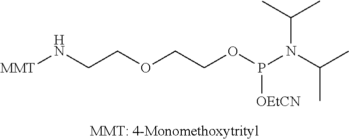

5′-amino modifier C12 (available from Glen Research cat#10-1912-90) of the structure:

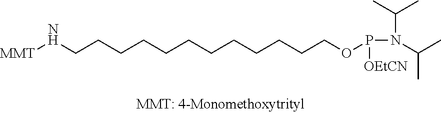

5′ thiol-modifier C6 (available from Glen Research cat#10-1926-90) of the structure:

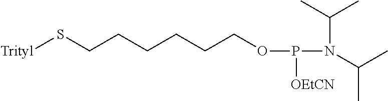

The 5′-thiol modified linker is used with PEG-maleimides, PEG-vinylsulfone, PEG-iodoacetamide and PEG-orthopyridyl-disulfide, for example.

In a particular embodiment, the oligonucleotide is bonded to the 5′-thiol through a maleimide or vinyl sulfone functionality.

The spacer may be, for example, a glycol spacer.

The oligonucleotide formulated according to the present invention may also be modified by encapsulation within a liposome.

### A. Aptamers

In one embodiment, the oligonucleotide formulated according to the present invention is an aptamer. Aptamers are short (i.e., typically 12-80 nucleotides in length) single-stranded nucleic acid polymers that bind with high affinity and specificity to targets. Potential targets for aptamers may include, for example, proteins, peptides, amino acids, small molecules, viruses and even whole cells. Meyer et al. Journal of Nucleic Acids, vol. 2011 (2011). The dissociation constants of aptamer-target complexes are comparable to those of antibodies and can reach the picomolar range. Due to their binding properties, aptamers have become useful tools for diagnostic and therapeutic applications. Keefe et al., Nature Reviews Drug Discovery, vol. 9, no. 7, pp. 537-550, 2010; Bouchard et al., Annual Review of Pharmacology and Toxicology, vol. 50, pp. 237-257, 2010.

In a particular embodiment, the aptamer formulated according to the present invention binds to a target protein.

Nucleic acid aptamers are isolated using the Systematic Evolution of Ligands by Exponential Enrichment (SELEX) process. This method allows the in vitro evolution of nucleic acid molecules with highly specific binding to target molecules. The SELEX method is described in, for example, U.S. Pat. No. 7,087,735, U.S. Pat. No. 5,475,096 and U.S. Pat. No. 5,270,163, (see also WO 91/19813).

The SELEX method involves selection from a mixture of candidate oligonucleotides and step-wise iterations of binding, partitioning and amplification, using the same general selection scheme, to achieve virtually any desired criterion of binding affinity and selectivity. Starting from a mixture of nucleic acids, such as mixtures comprising a segment of randomized sequence, the SELEX method includes steps of contacting the mixture with the target under conditions favorable for binding, partitioning unbound nucleic acids from those nucleic acids which have bound specifically to target molecules, dissociating the nucleic acid-target complexes, amplifying the nucleic acids dissociated from the nucleic acid-target complexes to yield a ligand-enriched mixture of nucleic acids, then reiterating the steps of binding, partitioning, dissociating and amplifying through as many cycles as desired to yield highly specific, high affinity aptamers to the target molecule.

The basic SELEX method has been modified to achieve a number of specific objectives. For example, U.S. Pat. No. 5,707,796 describes the use of SELEX in conjunction with gel electrophoresis to select nucleic acid molecules with specific structural characteristics, such as bent DNA. U.S. Pat. No. 5,763,177 describes a SELEX-based method for selecting aptamers containing photoreactive groups capable of binding and/or photocrosslinking to and/or photoinactivating a target molecule. U.S. Pat. No. 5,580,737 describes a method for identifying highly specific aptamers able to discriminate between closely related molecules, termed Counter-SELEX. U.S. Pat. Nos. 5,567,588 and 5,861,254 describe SELEX-based methods which achieve highly efficient partitioning between oligonucleotides having high and low affinity for a target molecule. U.S. Pat. No. 5,496,938, describes methods for obtaining improved aptamers after the SELEX process has been performed. U.S. Pat. No. 5,705,337, describes methods for covalently linking a ligand to its target.

The feasibility of identifying aptamers to small peptides in solution was demonstrated in U.S. Pat. No. 5,648,214. The ability to use affinity elution with a ligand to produce aptamers that are targeted to a specific site on the target molecule is exemplified in U.S. Pat. No. 5,780,228, which relates to the production of high affinity aptamers binding to certain lectins. Methods of preparing aptamers to certain tissues, which include groups of cell types, are described in U.S. Pat. No. 6,127,119. The production of certain modified high affinity ligands to calf intestinal phosphatase is described in U.S. Pat. No. 6,673,553. U.S. Pat. No. 6,716,580 describes an automated process of identifying aptamers that includes the use of robotic manipulators.

In its most basic form, the SELEX process may be defined by the following series of steps:

1) A candidate mixture of nucleic acids of differing sequence is prepared. The candidate mixture generally includes regions of fixed sequences (i.e., each of the members of the candidate mixture contains the same sequences in the same location) and regions of randomized sequences. The fixed sequence regions are selected either: (a) to assist in the amplification steps described below, (b) to mimic a sequence known to bind to the target, or (c) to enhance the concentration of a given structural arrangement of the nucleic acids in the candidate mixture. The randomized sequences can be totally randomized (i.e., the probability of finding a base at any position being one in four) or only partially randomized (e.g., the probability of finding a base at any location can be selected at any level between 0 and 100 percent).

2) The candidate mixture is contacted with the selected target under conditions favorable for binding between the target and members of the candidate mixture. Under these circumstances, the interaction between the target and the nucleic acids of the candidate mixture can be considered as forming nucleic acid-target pairs between the target and those nucleic acids having the strongest affinity for the target.

3) The nucleic acids with the highest affinity for the target are partitioned from those nucleic acids with lesser affinity to the target. Because only an extremely small number of sequences (and possibly only one molecule of nucleic acid) corresponding to the highest affinity nucleic acids exist in the candidate mixture, it is generally desirable to set the partitioning criteria so that a significant amount of the nucleic acids in the candidate mixture (approximately 5 to 50%) are retained during partitioning.

4) Those nucleic acids selected during partitioning as having the relatively higher affinity to the target are then amplified to create a new candidate mixture that is enriched in nucleic acids having a relatively higher affinity for the target.

5) By repeating the partitioning and amplifying steps above, the newly formed candidate mixture contains fewer and fewer weakly binding sequences, and the average degree of affinity of the nucleic acids to the target will generally increase. Taken to its extreme, the SELEX process will yield a candidate mixture containing one or a small number of unique nucleic acids representing those nucleic acids from the original candidate mixture having the highest affinity to the target molecule.

In one embodiment, the aptamer suitable for use in the present invention comprises from about 10 to about 50 nucleotides or from about 10 to about 15, about 15 to about 20, about 20 to about 25, about 25 to about 30, about 30 to about 35, about 35 to about 40, about 40 to about 45 or about 45 to about 50 nucleotides.

The aptamer formulated according to the present invention may be modified or unmodified, as described previously. In one embodiment, the aptamer comprises one or more modifications. The SELEX method encompasses the identification of high-affinity aptamers containing modified nucleotides conferring improved characteristics on the ligand, such as improved in vivo stability or improved delivery characteristics. Examples of such modifications include chemical substitutions at the ribose and/or phosphate and/or base positions. SELEX-identified aptamers containing modified nucleotides are described in U.S. Pat. No. 5,660,985 that describes oligonucleotides containing nucleotide derivatives chemically modified at the 5- and 2′-positions of pyrimidines. U.S. Pat. No. 5,580,737 describes specific aptamers containing one or more nucleotides modified with 2′-amino (2′—NH2), 2′-fluoro (2′-F), and/or 2′-O-methyl (2′-OMe). U.S. Pat. No. 5,756,703, describes oligonucleotides containing various 2′-modified pyrimidines.

The SELEX method encompasses combining selected oligonucleotides with other selected oligonucleotides and non-oligonucleotide functional units as described in U.S. Pat. Nos. 5,637,459 and 5,683,867. U.S. Pat. No. 5,637,459 describes highly specific aptamers containing one or more nucleotides modified with 2′-amino (2′—NH 2), 2′-fluoro (2′-F), and/or 2′-O-methyl (2′-OMe). The SELEX method further encompasses combining selected aptamers with lipophilic or non-immunogenic, high molecular weight compounds in a diagnostic or therapeutic complex as described in U.S. Pat. No. 6,011,020.

Where the aptamers are derived by the SELEX method, the modifications can be pre- or post-SELEX modifications. Pre-SELEX modifications can yield aptamers with both specificity for its target and improved in vivo stability. Post-SELEX modifications made to 2′-OH-containing aptamers can result in improved in vivo stability without adversely affecting the binding capacity of the aptamers. In one embodiment, the modifications of the aptamer include a 3′-3′ inverted phosphodiester linkage at the 3′ end of the molecule and 2′ fluoro (2′-F) and/or 2′ amino (2′—NH2), and/or 2′ O methyl (2′-OMe) modification of some or all of the nucleotides.

In one embodiment, the aptamer is conjugated to another molecule conferring desired biological properties, such as increased in vivo stability. The molecule conjugated to the aptamer may be, for example, a peptide, a protein, a carbohydrate, an antibody, a hybridization triggered cross-linking agent, a transport agent, hybridization-triggered cleavage agent, an enzyme, a polymer, a drug, a fluorophor, a lipophilic compound such as cholesterol, dialkyl glycerol, diacyl glycerol, or a non-immunogenic, high molecular weight compound or polymer such as polyethylene glycol (PEG) or other water soluble pharmaceutically acceptable polymers including, but not limited to, polyaminoamines (PAMAM); polysaccharides such as dextran, or polyoxazolines (POZ).

In a particular embodiment, the aptamer is conjugated to PEG, i.e., a pegylated aptamer. Numerous PEGs are known in the art, as described in Section II. In a particular embodiment, the aptamer is conjugated to PEG having a molecule weight of from about 20 kD to about 60 kD, about 20 kD, about 30 kD, about 40 kD, about 50 kD or about 60 kD. In a particular embodiment, the aptamer is conjugated to PEG having a molecular weight of about 40 kD. The aptamer may be conjugated to a single PEG or multiple PEGs.

The aptamer may be conjugated through covalent bonding or through non-covalent interactions, as described in Section II.

Attachment of the molecule to other components of the complex can be done directly or with the utilization of linkers or spacers, as described in Section II.

In a particular embodiment, the aptamer-PEG complex utilizes a hexylamino linker US Patent Application Publication No. US20120277419 describes the conjugation of an aptamer to PEG via a linker moiety.

In still other embodiments, the aptamer is encapsulated inside a liposome.

The aptamer can be modified at the base moiety, sugar moiety, or phosphate backbone, for example, to improve stability of the molecule, hybridization, etc. The aptamer may comprise at least one modified base selected from the group including, but not limited to, 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2.alpha.-thiouracil, beta.-D-mannosylqueosine, 5′-methoxycarboxymethyluracil, 5-methoxyuracil, 2-methylthio-N6 isopentenyladenine, uracil oxyacetic acid, wybutoxosine, pseudouracil, queosine, 2-thiocytosine, 5-methyl thiouracil, 2-thiouracil, 4-thiouracil, 5-methyluracil, -uracil-5-oxyacetic acid methylester, uracil oxyacetic acid (v), 5-methyl thiouracil, 3-(3-amino-3-N carboxypropyl) and 2,6-diaminopurine.

The aptamer may also comprise at least one modified sugar moiety selected from the group including, but not limited to, arabinose, 2-fluoroarabinose, xylose, and hexose. The aptamer or modulator can comprise at least one modified phosphate backbone selected from the group including, but not limited to, a phosphorothioate, a phosphorodithioate, a phosphoramidothioate, a phosphoramidate, a phosphorodiamidate, a methylphosphonate, an alkyl phosphotriester, and a formacetal or analog thereof.

### B. Modulators of Aptamers

In one embodiment, the aptamer formulated according to the present invention is an aptamer having binding activity that can be reversed or neutralized by a second agent (known variously as a modulator, regulator, control agent or antidote) capable of specifically binding a portion of the aptamer or otherwise modulating its activity (Rusconi et al., 2004, Nat. Biotechnol. 22(11):1423-8; Rusconi et al., 2002, Nature 419(6902):90-4). Together, the aptamer and the modulator constitute a therapeutic system.

The aptamer may be an unmodified or modified aptamer as described herein. The modulator may be an oligonucleotide modulator or a non-oligonucleotide modulator, such as a small molecule, peptide, oligosaccharide, for example an aminoglycoside, or other molecule that can specifically bind to or otherwise modulate the activity of the aptamer, or a chimera or fusion or linked product of any of these.

In one embodiment, the modulator is an oligonucleotide having a sequence that is complementary to or hybridizes with at least a portion of the aptamer. The oligonucleotide modulator may be modified or unmodified, as taught herein.

In another embodiment, the modulator is a ribozyme, DNAzyme, peptide nucleic acid (PNA), morpholino nucleic acid (MNA), locked nucleic acid (LNA) or pseudocyclic oligonucleobases (PCO) having a sequence that is complementary to or hybridizes with at least a portion of the aptamer.

An aptamer possesses an active tertiary structure which is dependent on formation of the appropriate stable secondary structure. Therefore, while the mechanism of formation of a duplex between a complementary oligonucleotide modulator of the invention and an aptamer is the same as between two short linear oligoribonucleotides, both the rules for designing such interactions and the kinetics of formation of such a product are impacted by the intramolecular aptamer structure. The rate of nucleation is important for formation of the final stable duplex, and the rate of this step is greatly enhanced by targeting the oligonucleotide modulator to single-stranded loops and/or single-stranded 3′ or 5′ tails present in the aptamer. For the formation of the intermolecular duplex to occur, the free energy of formation of the intermolecular duplex has to be favorable with respect to formation of the existing intramolecular duplexes within the targeted aptamer.

Modulators can be designed so as to bind a particular aptamer with a high degree of specificity and a desired degree of affinity. Modulators can be also be designed so that, upon binding, the structure of the aptamer is modified to either a more or less active form. For example, the modulator can be designed so that upon binding to the targeted aptamer, the three-dimensional structure of that aptamer is altered such that the aptamer can no longer bind to its target molecule or binds to its target molecule with less affinity.

Alternatively, the modulator can be designed so that, upon binding, the three dimensional structure of the aptamer is altered so that the affinity of the aptamer for its target molecule is enhanced. That is, the modulator can be designed so that, upon binding, a structural motif is produced in the aptamer so that the aptamer can bind to its target molecule.

In an alternative embodiment of the invention, the modulator itself is an aptamer. In this embodiment, an aptamer is first generated that binds to the desired therapeutic target. In a second step, a second aptamer that binds to the first aptamer is generated using the SELEX process described herein or other process, and modulates the interaction between the therapeutic aptamer and the target. In one embodiment, the second aptamer deactivates the effect of the first aptamer.

In other alternative embodiments, the aptamer which binds to the target is selected from the group consisting of a PNA, MNA, LNA or PCO and the modulator is an aptamer. Alternatively, the aptamer which binds to the target is selected from the group consisting of a PNA, MNA, LNA or PCO, and the modulator is a PNA. Alternatively, the aptamer which binds to the target is selected from the group consisting of a PNA, MNA, LNA or PCO, and the modulator is an MNA. Alternatively, the aptamer which binds to the target is selected from the group consisting of a PNA, MNA, LNA or PCO, and the modulator is an LNA. Alternatively, the aptamer which binds to the target is selected from the group consisting of a PNA, MNA, LNA or PCO, and the modulator is a PCO. Any of these can be used, as desired, in the naturally occurring stereochemistry or in non-naturally occurring stereochemistry or a mixture thereof. For example, in a preferred embodiment, the aptamer is in the D configuration, and in an alternative embodiment, the aptamer is in the L configuration.

In one embodiment, the modulator is an oligonucleotide that comprises a sequence complementary to at least a portion of the targeted aptamer sequence. For example, the modulator oligonucleotide can comprise a sequence complementary to 6-25 nucleotides of the targeted aptamer, typically, 8-20 nucleotides, more typically, 10-15 nucleotides. Advantageously, the modulator oligonucleotide is complementary to 6-25 consecutive nucleotides of the aptamer, or 8-20 or 10-15 consecutive nucleotides. The length of the modulator oligonucleotide can be optimized taking into account the targeted aptamer and the effect sought. Typically the modulator oligonucleotide is 5-80 nucleotides in length, more typically, 10-30 and most typically 15-20 nucleotides (e.g., 15-17). The oligonucleotide can be made with nucleotides bearing D or L stereochemistry, or a mixture thereof. Naturally occurring nucleosides are in the D configuration.

Various strategies can be used to determine the optimal site for oligonucleotide binding to a targeted aptamer. An empirical strategy can be used in which complimentary oligonucleotides are “walked” around the aptamer. A walking experiment can involve two experiments performed sequentially. A new candidate mixture can be produced in which each of the members of the candidate mixture has a fixed nucleic acid-region that corresponds to a oligonucleotide modulator of interest. Each member of the candidate mixture also contains a randomized region of sequences. According to this method it is possible to identify what are referred to as “extended” aptamers, which contain regions that can bind to more than one binding domain of an aptamer. In accordance with this approach, 2′-O-methyl oligonucleotides (e.g., 2′-O-methyl oligonucleotides) about 15 nucleotides in length can be used that are staggered by about 5 nucleotides on the aptamer (e.g., oligonucleotides complementary to nucleotides 1-15, 6-20, 11-25, etc. of aptamer the aptamer). An empirical strategy can be particularly effective because the impact of the tertiary structure of the aptamer on the efficiency of hybridization can be difficult to predict. Assays known in the art can be used to assess the ability of the different oligonucleotides to hybridize to a specific aptamer, with particular emphasis on the molar excess of the oligonucleotide required to achieve complete binding of the aptamer. See, e.g., PCT/US2002/16555. The ability of the different oligonucleotide modulators to increase the rate of dissociation of the aptamer from, or association of the aptamer with, its target molecule can also be determined by conducting standard kinetic studies using, for example, BIACORE assays. Oligonucleotide modulators can be selected such that a 5-50 fold molar excess of oligonucleotide, or less, is required to modify the interaction between the aptamer and its target molecule in the desired manner.

Alternatively, the targeted aptamer can be modified so as to include a single-stranded tail (3′ or 5′) in order to promote association with an oligonucleotide modulator. Suitable tails can comprise 1 to 20 nucleotides, preferably, 1-10 nucleotides, more preferably, 1-5 nucleotides and, most preferably, 3-5 nucleotides (e.g., modified nucleotides such as 2′-O-methyl sequences). Tailed aptamers can be tested in binding and bioassays (e.g., as described in the Examples that follow) to verify that addition of the single-stranded tail does not disrupt the active structure of the aptamer. A series of oligonucleotides (for example, 2′-O-methyl oligonucleotides) that can form, for example, 1, 3 or 5 base pairs with the tail sequence can be designed and tested for their ability to associate with the tailed aptamer alone, as well as their ability to increase the rate of dissociation of the aptamer from, or association of the aptamer with, its target molecule. Scrambled sequence controls can be employed to verify that the effects are due to duplex formation and not non-specific effects.

The oligonucleotide modulators comprise a sequence complementary to at least a portion of an aptamer. However, absolute complementarity is not required. A sequence “complementary to at least a portion of an aptamer,” referred to herein, means a sequence having sufficient complementarity to be able to hybridize with the aptamer. The ability to hybridize can depend on both the degree of complementarity and the length of the antisense nucleic acid. Generally, the larger the hybridizing oligonucleotide, the more base mismatches with a target aptamer it can contain and still form a stable duplex (or triplex as the case may be). One skilled in the art can ascertain a tolerable degree of mismatch by use of standard procedures to determine the melting point of the hybridized complex. In specific aspects, the oligonucleotide can be at least 5 or at least 10 nucleotides, at least 15 or 17 nucleotides, at least 25 nucleotides or at least 50 nucleotides. The oligonucleotides of the invention can be DNA or RNA or chimeric mixtures or derivatives or modified versions thereof, single-stranded.

Modulators can be identified in general, through binding assays, molecular modeling, or in vivo or in vitro assays that measure the modification of biological function, as would be understood by those of skill in the art and as described in the patents cited in this application that describe certain aptamers for specific applications. Standard binding assays can be used to identify and select modulators of the invention. In vivo or in vitro assays that evaluate the effectiveness of a regulator in modifying the interaction between a aptamer and a target are specific for the disorder being treated. There are ample standard assays for biological properties that are well known and can be used.

In one embodiment, the modulator has the ability to substantially bind to an aptamer in solution at modulator concentrations of less than one (1.0) micromolar (uM), preferably less than 0.1 uM, and more preferably less than 0.01 uM. By “substantially” is meant that at least a 50 percent reduction in target biological activity is observed by modulation in the presence of the a target, and at 50% reduction is referred to herein as an IC₅₀ value.

In an exemplary embodiment, oligonucleotides and oligonucleotide conjugates formulated according to the present invention include those disclosed in U.S. Pat. Nos. 7,312,325, 7,812,001, 7,741,307, 7,776,836, 7,858,591, 7,300,922, 7,304,041, 7,723,315, 7,531,524 and U.S. Patent Publications US-2009-20090048193, US-2010-0311820 and US 2012-0095085, the contents of which are incorporated by reference.

### C. Pegnivacogin

In a particular embodiment, the oligonucleotide conjugate formulated according to the present invention is pegnivacogin, also known as RB006 (SEQ ID NO: 1). Pegnivacogin is the drug component of REG1, an aptamer-based, anticoagulation system as described in US-2009-20090048193 and other patent publications cited herein.

Structurally, RB006 is a modified RNA aptamer, 31 nucleotides in length, which is stabilized against endonuclease degradation by the presence of 2′-fluoro and 2′-O-methyl sugar-containing residues, and stabilized against exonuclease degradation by a 3′ inverted deoxythymidine cap. The nucleic acid portion of the aptamer is conjugated via a hexylamino linker to a PEG having a molecular weight of 40 kD. As such, pegnivacogin has a molecular weight of approximately 50 kD (i.e., 31 nucleotides plus 40 kD PEG).

Functionally, RB006 is a direct FIXa inhibitor that binds coagulation factor IXa with high affinity and specificity (Rusconi et al., 2004, Nat. Biotechnol. 22(11):1423-8; Rusconi et al., 2002, Nature 419(6902):90-4; see also WO05/106042 and U.S. Pat. No. 7,723,315). It elicits an anticoagulant effect by blocking the FVIIIa/FIXa-catalyzed conversion of FX to FXa. It has a long half life due to its nuclease-stabilized backbone and the PEG component. When delivered intravenously, its half life is of about 96 hours. When delivered subcutaneously, its half life is greater than about 7 days.

In one embodiment, the activity of pegnivacogin is modulated by anivamersen, also known as RB007 (SEQ ID NO: 2). Anivamersen is a 2′-O-methyl RNA oligonucleotide 15 nucleotides in length that is complementary to a portion of RB006. The 2′-O-methyl modification confers moderate nuclease resistance to the modulator, which provides sufficient in vivo stability to enable it to seek and bind RB006, but does not support extended in vivo persistence.

### D. RB571

In a particular embodiment, the oligonucleotide conjugate formulated according to the present invention is known as RB571 (SEQ ID NO: 3). RB571 is the drug component of REG3, an aptamer-based, anti-platelet pharmacologic system as described in U.S. Pat. No. 8,318,923 and other patent publications cited herein.

In one embodiment, RB571 comprises a secondary structure, wherein the secondary structure comprises, in a 5′ to 3′ direction, a first stem region, a first loop region, a second stem region, a second loop region, a third loop region, a third stem region and a fourth loop region. In another embodiment, the RB571 consists essentially of, in a 5′ to 3′ direction, a first stem region, a first loop region, a second stem region, a second loop region, a third loop region, a third stem region and a fourth loop region.

Structurally, RB571 is a modified RNA aptamer, 29 nucleotides in length, which is stabilized against endonuclease degradation by the presence of 2′-fluoro and 2′-O-methyl sugar-containing residues, and stabilized against exonuclease degradation by a 3′-inverted deoxythymidine cap. The nucleic acid portion of the aptamer is conjugated via a hexylamino linker to a PEG having a molecular weight of 40 kD. As such, RB571 has a molecular weight of approximately 50 kD (i.e., 29 nucleotides plus 40 kD PEG).

Functionally, RB571 binds glycoprotein VI with high affinity and specificity (U.S. Pat. No. 8,318,923). While not to be limited by any particular mechanism, it is believed that RB571 elicits an antiplatelet effect by binding to glycoprotein VI on the surface of platelet cells which plays a key role in platelet adhesion and aggregation. It has a longer half life than an unmodified oligonucleotide due to its nuclease-stabilized backbone and the PEG component.

In one embodiment, the activity of RB571 is modulated by RB515 (SEQ ID NO 4). RB515 is a 2′-O-methyl RNA oligonucleotide 12 nucleotides in length that is complementary to a portion of RB571. The 2′-O-methyl modification confers moderate nuclease resistance to the modulator, which provides sufficient in vivo stability to enable it to seek and bind RB571, but does not support extended in vivo persistence.

RB571
(SEQ ID NO: 3)
(PEG40KGL2-NOF)(C6L)
mGmGmAmGmGAfCG(s)G(s)mCmG(6GLY)mCmG
fCfCfUmGmGfCmAfUmAmAmGmCmCmUmCmCiT
RB515
(SEQ ID NO: 4)
mUmUmAmUmGmCmCmAmGmGmCmG

Where the sequence abbreviations are reflected as follows: rG=2′Ribo G; rA=2′Ribo A; mG=2′O-Methyl G; mA=2′O-Methyl A; mC=2′O-Methyl C; mU=2′O-Methyl U; fC=2′Fluoro C; fU=2′Fluoro U; G=2′Deoxy G; A=2′Deoxy A; iT-inverted deoxythymidine; (s)-phosphorothioate linkage; (C6L)=hexylamino linker; (6GLY)=hexaethylene glycol linker ((incorporated using 9-O-Dimethoxytrityl-triethylene glycol,1-[(2-cyanoethyl)-(N,N-diisopropyl)]-phosphoramidite); (PEG40KGL2-NOF)=40 kDa Branched PEG (SUNBRIGHT GL2-400GS2 product); (6FAM): 6-carboxyfluorescein

### III. Antioxidants

In one embodiment, the pharmaceutical formulation of the present invention comprises an oligonucleotide or conjugated oligonucleotide and one or more antioxidants. The antioxidants for use in these oligonucleotide formulations include substances that when present at low concentrations compared to that of an oxidizable substrate would significantly delay or prevent oxidation of that substrate. It is well known that antioxidants do not totally prevent oxidation and may offer negligible benefit in isolation.

Antioxidants may be broadly grouped according to their mechanism of action: primary or chain breaking antioxidants and secondary or preventive antioxidants. Primary antioxidants act as free radical acceptors/scavengers and delay or inhibit the initiation step or interrupt the propagation step of autoxidation. Most of the primary antioxidants that act as chain breakers or free radical interceptors are mono- or polyhydroxy phenols with various ring substitutions.

In a particular embodiment, the antioxidant is a primary antioxidant.

Secondary or preventive antioxidants differ from primary antioxidants as secondary antioxidants do not convert free radicals into stable molecules. Rather, they act as chelators for prooxidant or catalyst metal ions, provide hydrogen to primary antioxidants, decompose hydroperoxide to nonradical species, deactivate singlet oxygen, absorb ultraviolet radiation, or act as oxygen scavengers. They often enhance the antioxidant activity of primary antioxidants.

In a particular embodiment, the antioxidant is a secondary antioxidant.

Compounds that exhibit secondary antioxidant activity by metal chelation include, for example, citric, malic, succinic and tartaric acids, ethylenediaminetetraacetic acid (EDTA) and phosphoric acid derivatives (e.g., polyphosphates and phytic acid). Other non-limiting examples of chelating agents that can be present in the oligonucleotide formulations include dicarboxymethyl-glutamic acid, ethylenediaminedisuccinic acid (EDDS), hepta sodium salt of diethylene triamine penta (methylene phosphonic acid) (DTPMP.Na7), malic acid, nitrilotriacetic acid (NTA), oxalic acid, phosphoric acid, polar amino acids (e.g., arginine, asparagine, aspartic acid, glutamic acid, glutamine, lysine, and ornithine), siderophores (e.g., Desferrioxamine B), succinic acid and combinations thereof.

In a particular embodiment, the antioxidant is a chelator.

Compounds that exhibit secondary antioxidant activity by oxygen scavenging and reducing agents include, for example, ascorbic acid, acorbyl palmitate, erythorbic acid, sodium erythorbate and sulfites.

Compounds that exhibit secondary antioxidant activity by singlet oxygen quenching include, for example, carotenoids (e.g., b-carotene, lycopene and lutein).

In one embodiment, the antioxidant is a reducing agent.

In a particular embodiment the reducing agent is a free radical-scavenging amino acid. In another embodiment, the free radical-scavenging amino acid is histidine, cysteine, tryptophan, or methionine. The antioxidant may be, for example, selected from thiols (including but not limited to cysteine, glutathione, thioglycerol) or thioethers (including but not limited to methionine, D-methionine, L-methionine, D-ethionine, L-ethionine, 3-methylthio-1,2-propanediol, methyl-3-(methylthio)propionate, 2-(ethylthio)ethylamine, 2-(methylthio)-ethanol, buthionine, S-methyl-L-cysteine, S-methyl-D-cysteine, D-methioninol and L-methioninol, diallyl sulfides and methylthioethane). WO 97/14430 discloses use of hydrophilic thioethers as antioxidants to prolong storage stability of aqueous formulations of proteins and peptides.

In another embodiment, the antioxidant is a chain terminator. Monothioglycerol provides one non-limiting example of a chain terminator that can be present in the oligonucleotide formulation.

In a particular embodiment, the antioxidant is methionine.

In another particular embodiment, the antioxidant is not ascorbic acid.

Other suitable antioxidants for use in the pharmaceutical formulation of the present invention include, without limitation, acetone sodium bisulfite, ascorbyl palmitate bisulfite sodium, butylated hydroxy anisole, butylated hydroxy toluene, cystein, cysteinate HCl, dithionite sodium, gentisic acid, gentisic acid ethanolamine, glutamate monosodium, hydrophosphorous acid formaldehyde sulfoxylate sodium, metabisulfite potassium, metabisulfite sodium, monothioglycerol, propyl gallate, sulfite sodium, and thioglycolate sodium.

### IV. Other Components

The pharmaceutical formulation of the present invention may contain one or more additional components. Such additional ingredients, of course, should not adversely affect the overall stability of the pharmaceutical formulation of the present invention.

In one embodiment, the pharmaceutical formulation contains an isotonicity agent. Isotonicity agents are well known in the art and include, for example, mannitol, NaCl, glucose and sucrose.

The pharmaceutical formulation of the present invention may include one or more buffering agents, for example one or more pharmaceutically acceptable buffering agents.

“Buffering agent” is intended to mean a compound used to resist change in pH upon dilution or addition of acid or alkali. Such compounds include, by way of example and without limitation, acetic acid, sodium acetate, adipic acid, benzoic acid, sodium benzoate, citric acid, maleic acid, monobasic sodium phosphate, dibasic sodium phosphate, lactic acid, tartaric acid, glycine, potassium metaphosphate, potassium phosphate, monobasic sodium acetate, sodium bicarbonate, sodium tartrate and sodium citrate anhydrous and dihydrate and others known to those of ordinary skill in the art. As discussed further below, the formulation may also include dissolved oxygen. Dissolved oxygen is not added to the formulation, as such, but rather is present naturally unless removed.

### V. Formulation

The pharmaceutical formulation of the present invention is preferably an aqueous formulation containing the oligonucleotide or oligonucleotide conjugate and optionally, at least one antioxidant.

Although water (e.g. water for injection or WFI) is the preferred solvent, other solvents may be utilized including a mixture of water and of one or more other water-miscible solvent(s).

In a preferred embodiment, the pharmaceutical formulation of the present invention is a pharmaceutical formulation utilizing water or other solvents including, mixtures of water with other pharmaceutically acceptable solvents, such as pharmaceutically acceptable alcohols.

The concentration of the oligonucleotide or conjugated oligonucleotide (e.g., pegylated aptamer) in solution may vary. In one embodiment, the concentration of oligonucleotide or oligonucleotide conjugate (e.g., pegylated aptamer) is about 1 mg/mL to about 500 mg/mL. In one embodiment, the concentration of oligonucleotide or oligonucleotide conjugate (e.g., pegylated aptamer) is about 1 mg/mL to about 300 mg/mL. In one embodiment, the concentration of oligonucleotide or oligonucleotide conjugate is about 1 mg/mL to about 200 mg/mL (e.g., pegyated aptamer). In one embodiment, the concentration of oligonucleotide or oligonucleotide conjugate (e.g., pegylated aptamer) is about 1 mg/mL to about 100 mg/mL.

In a particular embodiment, the concentration of the oligonucleotide or oligonucleotide conjugate (e.g., pegylated aptamer) is about 1 to about 10, about 10 to about 20, about 20 to about 30, about 30 to about 40, about 50 to about 60, about 70 to about 80, about 90 to about 100, about 100 to about 110, about 110 to about 120, about 120 to about 130, about 130 to about 140, about 140 to about 150, about 150 to about 160, about 160 to about 170, about 170 to about 180, about 180 to about 190, about 190 to about 200, about 200 to about 210, about 210 to about 220, about 220 to about 230, about 230 to about 240, about 240 to about 250, about 250 to about 260, about 260 to about 270, about 270 to about 280, about 280 to about 290, about 290 to about 300 mg/mL.

In a specific embodiment, the concentration of the oligonucleotide or oligonucleotide conjugate (e.g., pegylated aptamer) is about 21, about 22, about 23, about 24, about 25, about 26, about 27, about 28, about 29 or about 30 mg/mL. In one embodiment, the concentration of the oligonucleotide or oligonucleotide conjugate is about 24 mg/mL.

In a particular embodiment, the conjugated oligonucleotide is a pegylated aptamer and its concentration in solution is from about 1 to about 100, about 10 to about 50, about 10 to about 30, about 20 to about 30, and more particularly, about 20 to about 22, about 22 to about 24, about 24 to about 26, about 26 to about 30 or about 30 mg/mL.

In a specific embodiment, the pegylated aptamer is pegnivacogin and its concentration in solution is about 20 to about 30, about 20 to about 22, about 22 to about 24, about 24 to about 26, about 26 to about 28, about 28 to about 30 or more specifically, about 24 mg/mL.

In a specific embodiment, the pegylated aptamer is RB571 and its concentration in solution is about 1 to about 300, about 1 to about 100, about 10 to about 50, about 10 to about 30, about 20 to about 40, about 10 mg/mL, about 15 mg/mL, about 20 mg/mL, about 25 mg/mL, about 30 mg/mL, about 35 mg/mL or about 40 mg/mL.

The formulation may contain additional components, as discussed herein. The concentration of the isotonicity agent, if present, may vary. The isotonic agents may be used in quantities which impart to the formulation the same osmotic pressure as body fluid. The precise amount necessary to achieve the desired effect may depend on factors familiar to those of skill in the art, such as the concentration of the oligonucleotide therapeutic agent in the formulation.

For example, the concentration of the isotonicity agent may be, for example, about 0.01% to 10% (w/v) or about 0.1 to about 100 mg/mL, for example about 0.5 to about 7 mg/mL, for example about 1 to about 5 mg/mL.

In a particular embodiment, mannitol is present in an amount from about 0.5% to about 7.5% (w/v), more preferably about 4.0% to about 5.5% (w/v), for example about 5.0% (w/v) or about 5 to about 75 mg/mL, for example about 40 to about 55 mg/mL.

In another particular embodiment, NaCL is present in an amount from about 0.05% to about 1.2% (w/v), more preferably from about 0.08% to about 1% (w/v), for example about 0.9% (w/v) or an amount of about 0.5 to about 12 mg/mL, for example about 8 to about 10 mg/mL, for example about 3.6 mg/mL.

The concentration of buffering agents, if present, may vary. In one embodiment, the concentration is about 50 mM to about 80 mM, about 55 mM to about 75 mM, or about 60 mM to about 70 mM. In another embodiment, the concentration is about 50 mM, about 55 mM, about 60 mM, about 65 mM, about 70 mM, about 75 mM, about 80 mM, about 85 mM about 90 mM or about 100 mM. In a particular embodiment, the concentration of buffering agent is about 66 mM.

### A. Modified or Reduced pH

In one embodiment, the pharmaceutical formulation of the present invention has a modified (e.g. reduced) pH relative to formulations of oligonucleotide conjugates known in the art.

In one embodiment, the pH of the pharmaceutical formulation is about 7 or less. In a particular embodiment, the pH of the formulation is between about 7.0 and 6.5 or about 6.5 and about 6.0. In another particular embodiment, the pH of the formulation is about 7.0, about 6.9 about 6.8, about 6.7, about 6.6, about 6.5, about 6.4, about 6.3, about 6.2, about 6.1 or about 6.0.

In a particular embodiment, the pH of the pharmaceutical formulation is about 6.8 or less. In another particular embodiment, the pH of the pharmaceutical formulation is about 6.8.

In one embodiment, the present invention is a pharmaceutical formulation comprising a conjugated oligonucleotide (e.g., a conjugated aptamer), wherein the formulation has a pH of about 7 or less.

In a preferred embodiment, the present invention is a pharmaceutical formulation comprising pegnivacogin, wherein the formulation has a pH of about 7.0 or less and more particularly, about 6.8.

In another preferred embodiment, the present invention is a pharmaceutical formulation comprising RB571, wherein the formulation has a pH or about 7.0 or less.

In an exemplary embodiment, the present invention is a pharmaceutical formulation comprising pegnivacogin, wherein the formulation has a pH of about 7.0 or less and more particularly, about 6.8, and wherein the formulation has a shelf life of at least about 24 months or more preferably, at least about 36 months under varied storage conditions. For example, the storage conditions may vary by temperature and include, without limitation, temperature ranges of about 2° C. to about 8° C., about 8° C. to about 24° C. or about 25° C. to about 30° C.

In other embodiments of the present invention, the pH of the formulation is not modified or reduced. In one embodiment, the pH is greater than about 7. In one embodiment, the pH is about 7.4 or greater.

### B. Dissolved Oxygen Levels

The dissolved oxygen levels (D0₂) of the pharmaceutical formulation of the present invention may be ambient or modified (e.g., reduced).

In one embodiment, the dissolved oxygen level is ambient. Without treatment to reduce dissolved oxygen, the dissolved oxygen level is between about 5 and about 10 ppm. In one embodiment, the dissolved oxygen level is between about 8 and about 10 ppm. In another embodiment, the dissolved oxygen level is about 10 ppm.

In a particular embodiment, the present invention is a pharmaceutical formulation comprising pegnivacogin, wherein the formulation has an ambient level of dissolved oxygen, or more particularly, between about 5 and about 10 ppm.

In another embodiment, the formulation of the present invention has a modified (e.g., reduced) level of dissolved oxygen. The dissolved oxygen level may be modified (e.g., reduced) to the desired level by any suitable method known in the art. For example, the level of dissolved oxygen may be reduced by inert gas purging, as described further herein or would be familiar to those of skill in the art.

The modified or reduced level of dissolved oxygen of the pharmaceutical formulation may be, for example, between about 5.0 and about 4.5, about 4.5 and about 4.0, about 4.0 and about 3.5, about 3.5 and about 3.0, about 3.0 and about 2.5, about 2.5 and about 2.0, about 2.0 and about 1.5 ppm, about 1.0 ppm and about 0.5 ppm, about 0.5 ppm and about 0.1 ppm, about 0.1 ppm and about 0 ppm. In a particular embodiment, the level of dissolved oxygen is less than about 2.0, about 1.9, about 1.8, about 1.7, about 1.6, about 1.5, about 1.4, about 1.3, about 1.2, about 1.1, about 1.0, about 0.9, about 0.8, about 0.7, about 0.6, about 0.5, about 0.4, about 0.3, about 0.2, about 0.1 or about 0 ppm. In a particular embodiment, the dissolved oxygen level is less than about 0 ppm or sub ppm.

In a particular embodiment, the dissolved oxygen level of the pharmaceutical formulation is less than about 5 ppm. In another particular embodiment, the dissolved oxygen level is less than about 4 ppm. In another particular embodiment, the dissolved oxygen level is less than about 3 ppm. In another particular embodiment, the dissolved oxygen level is less than about 2 ppm. In another particular embodiment, the dissolved oxygen level is less than about 1 ppm.

In one embodiment, the present invention is a pharmaceutical formulation comprising a conjugated oligonucleotide such as a pegylated aptamer, wherein the formulation has a level of dissolved oxygen that is less than about 5 ppm, about 4 ppm, about 3 ppm, about 2 ppm or about 1 ppm.

In one embodiment, the present invention is a pharmaceutical formulation comprising pegnivacogin, wherein the formulation has a level of dissolved oxygen that is less than about 5 ppm, about 4 ppm, about 3 ppm, about 2 ppm, about 1 ppm or about 0 ppm.

In one embodiment, the present invention is a pharmaceutical formulation comprising RB571, wherein the formulation has a level of dissolved oxygen that is less than about 5 ppm, about 4 ppm, about 3 ppm, about 2 ppm, about 1 ppm or about 0 ppm.

C. Reduced pH/Dissolved Oxygen

The pharmaceutical formulation of the present invention may have a reduced pH and an ambient level of dissolved oxygen or, alternatively, a modified or reduced level of dissolved oxygen.

In one embodiment, the present invention is a pharmaceutical formulation comprising an oligonucleotide or oligonucleotide conjugate (e.g., a pegylated aptamer), wherein the formulation has a reduced pH and an ambient level of dissolved oxygen.

In a particular embodiment, the pharmaceutical formulation comprises a pegylated aptamer, wherein the formulation has a pH of about 7 or less, or more particularly, about 6.8, and a level of dissolved oxygen between about 5 and about 10 ppm.

In a preferred embodiment, the pharmaceutical formulation of the present invention comprises pegnivacogin, wherein the formulation has a pH of about 7 or less, or more particularly, about 6.8, and an ambient level of dissolved oxygen, or more particularly, a dissolved oxygen level of between about 5 and about 10 ppm. This exemplary formulation has a shelf life, for example, of at least about 24 months, or more preferably, at least about 36 months under variable storage conditions, including for example, storage temperatures between about 2° C. to about 8° C., about 8° C. to about 24° C., or about 25° C. to about 30° C.

In a preferred embodiment, the pharmaceutical formulation of the present invention comprises RB571, wherein the formulation has a pH of about 7 or less, or more particularly, about 6.8, and an ambient level of dissolved oxygen, or more particularly, a dissolved oxygen level of between about 5 and about 10 ppm. This exemplary formulation has a shelf life, for example, of at least about 24 months, or more preferably, at least about 36 months under variable storage conditions, including for example, storage temperatures between about 2° C. to about 8° C., about 8° C. to about 24° C., or about 25° C. to about 30° C.

In another embodiment, the present invention is a pharmaceutical formulation comprising an oligonucleotide or oligonucleotide conjugate, e.g., a pegylated aptamer, having a reduced pH and a reduced level of dissolved oxygen.

In a particular embodiment, the pharmaceutical formulation comprises a pegylated aptamer and has a pH of about 7 or less and a dissolved oxygen level of less than about 5 ppm or about 2 ppm.

In a further particular embodiment, the pharmaceutical formulation comprises a pegylated aptamer and has a pH of about 6.8 or less and a dissolved oxygen level of about 5 ppm or about 2 ppm or less.

In yet another particular embodiment, the pharmaceutical formulation comprises a pegylated aptamer and has a pH of about 7 or less and a dissolved oxygen level of about 1 ppm or less.

In a specific embodiment, the present invention is a pharmaceutical formulation comprising an oligonucleotide or oligonucleotide conjugate, e.g., a pegylated aptamer, having a pH of about 7 or less and dissolved oxygen content of less than about 5 ppm and a shelf life of at least about 24 months, or, more preferably, at least about 36 months, under varied conditions including, for example, when stored at a temperature from about 2° C. to about 8° C., about 8° C. to about 24° C. or about 25° C. to about 30° C.

In a specific embodiment, the present invention is a pharmaceutical formulation comprising an oligonucleotide or oligonucleotide conjugate, e.g., a pegylated aptamer, having a pH or about 7 or less and dissolved oxygen content of about 2 ppm or less, with a shelf life of at least about 24 months, or, more preferably, at least about 36 months from about 2° C. to about 8° C.

### D. Antioxidants

In certain embodiments, the pharmaceutical formulation of the present invention comprises oligonucleotide or a conjugated oligonucleotide, such as pegylated oligonucleotide or pegylated aptamer, and at least one antioxidant.

Compounds that exhibit secondary antioxidant activity by metal chelation include, for example, citric, malic, succinic and tartaric acids, ethylenediaminetetraacetic acid (EDNA) and phosphoric acid derivatives (e.g., polyphosphates and phytic acid). Other non-limiting examples of chelating agents that can be present in the oligonucleotide formulations include dicarboxymethyl-glutamic acid, ethylenediaminedisuccinic acid (ADDS), hepta sodium salt of diethylene triamine penta (methylene phosphonic acid) (DTPMP.Na7), malic acid, nitrilotriacetic acid (NTA), oxalic acid, phosphoric acid, polar amino acids (e.g., arginine, asparagine, aspartic acid, glutamic acid, glutamine, lysine, and ornithine), siderophores (e.g., Desferrioxamine B), succinic acid and combinations thereof.

In a particular embodiment, the antioxidant is a chelator.

Compounds that exhibit secondary antioxidant activity by oxygen scavenging and reducing agents include, for example, ascorbic acid, acorbyl palmitate, erythorbic acid, sodium erythorbate and sulfites.

Compounds that exhibit secondary antioxidant activity by singlet oxygen quenching include, for example, carotenoids (e.g., b-carotene, lycopene and lutein).

In one embodiment, the antioxidant is a reducing agent.

In a particular embodiment the reducing agent is a free radical-scavenging amino acid. In another embodiment, the free radical-scavenging amino acid is histidine, cysteine, tryptophan, or methionine. The antioxidant may be, for example, selected from thiols (including but not limited to cysteine, glutathione, thioglycerol) or thioethers (including but not limited to methionine, D-methionine, L-methionine, D-ethionine, L-ethionine, 3-methylthio-1,2-propanediol, methyl-3-(methylthio)propionate, 2-(ethylthio)ethylamine, 2-(methylthio)-ethanol, buthionine, S-methyl-L-cysteine, S-methyl-D-cysteine, D-methioninol and L-methioninol, diallyl sulfides and methylthioethane). WO 97/14430 discloses use of hydrophilic thioethers as antioxidants to prolong storage stability of aqueous formulations of proteins and peptides.

In another embodiment, the antioxidant is a chain terminator. Monothioglycerol provides one non-limiting example of a chain terminator that can be present in the oligonucleotide formulation.

In a particular embodiment, the antioxidant is methionine.

In another particular embodiment, the antioxidant is not ascorbic acid.

Other suitable antioxidants include, without limitation, acetone sodium bisulfite, ascorbyl palmitate bisulfite sodium, butylated hydroxy anisole, butylated hydroxy toluene, cystein, cysteinate HCl, dithionite sodium, gentisic acid, gentisic acid ethanolamine, glutamate monosodium, hydrophosphorous acid formaldehyde sulfoxylate sodium, metabisulfite potassium, metabisulfite sodium, monothioglycerol, propyl gallate, sulfite sodium, and thioglycolate sodium.

In a particular embodiment, the present invention is a pharmaceutical formulation comprising a conjugated oligonucleotide and at least one antioxidant, which formulation has a shelf life of at least about 24 months, or, more preferably, at least about 36 months under varied storage conditions including, for example, at about 2° C. to about 8° C., about 8° C. to about 24° C. or about 25° C. to about 30° C.

The concentration of at least one antioxidant may vary. In a particular embodiment, the present invention is a pharmaceutical formulation comprising an oligonucleotide or a conjugated oligonucleotide (e.g., a pegylated aptamer) and from about 0.10 to about 0.15, about 0.15 to about 0.20, about 0.20 to about 0.25, about 0.30 to about 0.35, about 0.40 to about 0.45, or about 0.45 to about 0.50% w/w of an antioxidant.

The concentration of the antioxidant may be lower than 0.1% w/w. In a particular embodiment, the present invention is a pharmaceutical formulation comprising an oligonucleotide or a conjugated oligonucleotide and from about 0.01 to about 0.1, about 0.10 to about 0.15, about 0.15 to about 0.20, about 0.20 to about 0.25, about 0.30 to about 0.35, about 0.40 to about 0.45, or about 0.45 to about 0.50% w/w of an antioxidant.

In a particular embodiment, the present invention is a pharmaceutical formulation comprising an oligonucleotide or a conjugated oligonucleotide and from about 0.001 to about 0.1, about 0.10 to about 0.15, about 0.15 to about 0.20, about 0.20 to about 0.25, about 0.30 to about 0.35, about 0.40 to about 0.45, or about 0.45 to about 0.50% w/w of a reducing agent.

In a further particular embodiment, the present invention is a pharmaceutical formulation comprising an oligonucleotide or a conjugated oligonucleotide (e.g., a pegylated aptamer) and from about 0.001 to about 0.1, about 0.10 to about 0.15, about 0.15 to about 0.20, about 0.20 to about 0.25, about 0.30 to about 0.35, about 0.40 to about 0.45, or about 0.45 to about 0.50% w/w of methionine.

In yet another particular embodiment, the present invention is a pharmaceutical formulation comprising from about 1 to about 100 mg/mL oligonucleotide or a conjugated oligonucleotide (e.g., a pegylated aptamer) and from about 0.001 to about 0.1, about 0.10 to about 0.15, about 0.15 to about 0.20, about 0.20 to about 0.25, about 0.30 to about 0.35, about 0.40 to about 0.45, or about 0.45 to about 0.50% w/w of methionine.

In an exemplary embodiment, the present invention is a pharmaceutical formulation comprising from about 1 to about 100 mg/mL pegnivacogin and from about 0.001 to about 0.10, about 0.10 to about 0.15, about 0.15 to about 0.20, about 0.20 to about 0.25, about 0.30 to about 0.35, about 0.40 to about 0.45, or about 0.45 to about 0.50% w/w of methionine.

In another exemplary embodiment, the present invention is a pharmaceutical formulation comprising from about 20 to about 30 mg/mL pegnivacogin and from about 0.001 to about 0.10, about 0.10 to about 0.15, about 0.15 to about 0.20, about 0.20 to about 0.25, about 0.30 to about 0.35, about 0.40 to about 0.45, or about 0.45 to about 0.50% w/w of methionine.

In a still further specific embodiment, the present invention is a pharmaceutical formulation comprising about 24 mg/mL pegnivacogin and from about 0.001 to about 0.10, about 0.10 to about 0.15, about 0.15 to about 0.20, about 0.20 to about 0.25, about 0.30 to about 0.35, about 0.40 to about 0.45, or about 0.45 to about 0.50% w/w of methionine.

In a preferred embodiment, the present invention is a pharmaceutical formulation comprising about 24 mg/mL pegnivicogin and about 0.10% w/w methionione.

In a still further specific embodiment, the present invention is a pharmaceutical formulation comprising about 24 mg/mL RB571 and from about 0.001 to about 0.10, about 0.10 to about 0.15, about 0.15 to about 0.20, about 0.20 to about 0.25, about 0.30 to about 0.35, about 0.40 to about 0.45, or about 0.45 to about 0.50% w/w of methionine.

In a preferred embodiment, the present invention is a pharmaceutical formulation comprising about 24 mg/mL RB571 and about 0.10% w/w methionione.

E. Formulations Containing Antioxidants and Modified or Reduced pH and/or Ambient or Modified Dissolved Oxygen Levels

In certain embodiments, the pharmaceutical formulation comprises an oligonucleotide or oligonucleotide conjugate (e.g., a pegylated aptamer) and at least one antioxidant (e.g., methionine), wherein the formulation has a modified or reduced pH and/or an ambient or modified (e.g., reduced) dissolved oxygen level.

In one embodiment, the present invention is a pharmaceutical formulation comprising an oligonucleotide or conjugated oligonucleotide (e.g., a pegylated aptamer) and at least one antioxidant (e.g., methionine), wherein the formulation has a modified or reduced pH.

In another embodiment, the present invention is a pharmaceutical formulation comprising an oligonucleotide or conjugated oligonucleotide (e.g., a pegylated aptamer) and at least one antioxidant (e.g., methionine) wherein the formulation has an ambient dissolved oxygen level or a modified or reduced dissolved oxygen level, e.g., less than about 5 ppm.

In yet another embodiment, the present invention is a pharmaceutical formulation comprising an oligonucleotide or conjugated oligonucleotide (e.g., a pegylated aptamer), wherein the formulation has a pH of about 7 or less and/or an ambient dissolved oxygen level or a modified or reduced level of dissolved oxygen, e.g., less than about 5 ppm.

In a particular embodiment, the present invention is a pharmaceutical formulation comprising an oligonucleotide or a conjugated oligonucleotide (e.g., a pegylated aptamer) and from about 0.10 to about 0.15, about 0.15 to about 0.20, about 0.20 to about 0.25, about 0.30 to about 0.35, about 0.40 to about 0.45, or about 0.45 to about 0.50% w/w of at least one antioxidant (e.g., methionine), wherein the formulation has a pH or about 7 or less and/or a level of dissolved oxygen that is either ambient or reduced, wherein the reduced dissolved oxygen level is less than about 5 ppm, about 4 ppm, about 3 ppm about 2 ppm, between about 2.0 and about 1.5 ppm, about 1.0 ppm and about 0.5 ppm, about 0.5 ppm and about 0.1 ppm, about 0.1 ppm and about 0 ppm.

In another particular embodiment, the present invention is a pharmaceutical formulation comprising an oligonucleotide or a conjugated oligonucleotide (e.g., a pegylated aptamer) and from about 0.001 to about 0.1, about 0.005 to about 0.05, about 0.01 to about 0.05% w/w or about 0.001, 0.01, 0.025, 0.05 or 0.1% w/w of at least one antioxidant (e.g., methionine); wherein the formulation has a pH of about 7 or less and/or a level of dissolved oxygen that is ambient or reduced, wherein the reduced dissolved oxygen level is less than about 5.0, about 3.0, about 2.0, about 1.9, about 1.8, about 1.7, about 1.6, about 1.5, about 1.4, about 1.3, about 1.2, about 1.1, about 1.0, about 0.9, about 0.8, about 0.7, about 0.6, about 0.5, about 0.4, about 0.3, about 0.2, about 0.1 or about 0 ppm.

In a further particular embodiment, the present invention is a pharmaceutical formulation comprising a conjugated aptamer (e.g., a pegylated aptamer) and from about 0.001 to about 0.1, about 0.005 to about 0.05, about 0.01 to about 0.05% wt/wt or about 0.001, 0.01, 0.025, 0.05 or 0.1% w/w of at least one antioxidant (e.g., methionine); wherein the formulation has a pH of about 7 or less and/or a level of dissolved oxygen that is ambient or reduced, wherein the reduced dissolved oxygen level is less than about 5.0, about 4.0, about 3.0, about 2.0, about 1.9, about 1.8, about 1.7, about 1.6, about 1.5, about 1.4, about 1.3, about 1.2, about 1.1, about 1.0, about 0.9, about 0.8, about 0.7, about 0.6, about 0.5, about 0.4, about 0.3, about 0.2, about 0.1 or about 0 ppm.

In another particular embodiment, the present invention is a pharmaceutical formulation comprising pegnivacogin and from about 0.001 to about 0.1, about 0.005 to about 0.05, about 0.01 to about 0.05 w/w or about 0.001, 0.01, 0.025, 0.05 or 0.1% wt./wt. of at least one antioxidant (e.g., methionine), and wherein the formulation has a pH of about 7.0 or less and/or a level of dissolved oxygen that is ambient or reduced, wherein the reduced dissolved oxygen level is less than about 5.0, about 4.0, about 3.0, about 2.0, about 1.9, about 1.8, about 1.7, about 1.6, about 1.5, about 1.4, about 1.3, about 1.2, about 1.1, about 1.0, about 0.9, about 0.8, about 0.7, about 0.6, about 0.5, about 0.4, about 0.3, about 0.2, about 0.1 or about 0 ppm.

In yet another particular embodiment, the present invention is a pharmaceutical formulation comprising pegnivacogin and from about 0.001 to about 0.1, about 0.005 to about 0.05, about 0.01 to about 0.05 w/w or about 0.001, 0.01, 0.025, 0.05 or 0.1% wt./wt. of at least one antioxidant (e.g., methionine), and wherein the formulation has a pH of about 7.0 or less and a level of dissolved oxygen that is ambient or more particularly, between about 5 and about 10 ppm.

In an exemplary embodiment, the present invention is a pharmaceutical formulation comprising from about 1 to about 100 mg/mL, or more particularly about 20 to about 30 mg/mL pegnivacogin and from about 0.001 to about 0.1, about 0.005 to about 0.05, about 0.01 to about 0.05 w/w or about 0.001, 0.01, 0.025, 0.05 or 0.1% wt./wt. of methionine, wherein the formulation has a pH of 7.0 or less and a dissolved oxygen level that is ambient or more particularly, about 5 to about 10 ppm.

In another exemplary embodiment, the present invention is a pharmaceutical formulation comprising from about 1 to about 100 mg/mL, or more particularly, about 20 to about 30 mg/mL pegnivacogin and from about 0.001 to about 0.1, about 0.005 to about 0.05, about 0.01 to about 0.05 w/w or about 0.001, 0.01, 0.025, 0.05 or 0.1% wt./wt. of methionine, wherein the formulation has a pH of 7.0 or less and a dissolved oxygen level that is reduced, or more particularly, less than about 5.0, about 4.0, about 3.0, about 2.0, about 2.0, about 1.9, about 1.8, about 1.7, about 1.6, about 1.5, about 1.4, about 1.3, about 1.2, about 1.1, about 1.0, about 0.9, about 0.8, about 0.7, about 0.6, about 0.5, about 0.4, about 0.3, about 0.2, about 0.1 or about 0 ppm.

In a specific embodiment, the present invention is a pharmaceutical formulation comprising about 24 mg/mL of pegnivacogin and from about 0.025 to about 0.1% w/w of methionine, wherein the formulation has a pH of about 7.0 or less and a level of dissolved oxygen that is ambient or reduced, wherein the reduced dissolved oxygen level is less than about 5.0 ppm.

In a preferred embodiment, the present invention is a pharmaceutical formulation In a preferred embodiment, the present invention is a pharmaceutical formulation comprising about 24 mg/mL of pegnivacogin and about 0.1% w/w of methionine, wherein the formulation has a pH of about 7.0 or less, or more particularly, about 6.8, and a level of dissolved oxygen that is ambient or more particularly, a level of dissolved oxygen between about 5 and about 10 ppm.

In another preferred embodiment, the present invention is a pharmaceutical formulation. In a preferred embodiment, the present invention is a pharmaceutical formulation comprising about 24 mg/mL of pegnivacogin and about 0.1% w/w of methionine, wherein the pH is about 7.0 or less, or more particularly about 6.8, and the level of dissolved oxygen is ambient or between about 5 and about 10 ppm and wherein the formulation has a shelf life of at least about 24 months and preferably, at least about 36 months under variable storage conditions including, for example, temperature of about 2° C. to about 8° C., about 8° C. to about 24° C. or about 25° C. to about 30° C.

In a still further preferred embodiment, the present invention is a pharmaceutical formulation In a preferred embodiment, the present invention is a pharmaceutical formulation comprising about 24 mg/mL of pegnivacogin and about 0.1% w/w of methionine, wherein the pH is about 7.0 or less, or more particularly about 6.8, and the level of dissolved oxygen is ambient or between about 5 and about 10 ppm and wherein the formulation has a shelf life that is enhanced over conjugated oligonucleotide formulations known in the art or the formulation disclosed in Example 1 by about 5, about 10, about 15, about 20, about 25, about 30, about 35, about 40, about 45, about 50, about 55, about 60, about 65, about 70, or about 75% or greater under variable storage conditions including, but not limited to, storage at a temperature of about 2° C. to about 8° C., about 8° C. to about 24° C. or about 25° C. to about 30° C.

In yet another preferred embodiment, the present invention is a pharmaceutical formulation comprising about 24 mg/mL of pegnivacogin and about 0.1% w/w of methionine, wherein the formulation has a pH of about 7.0 or less, or more particularly about 6.8, and a level of dissolved oxygen that is reduced and more particularly, the level of dissolved oxygen less than about 5.0, about 4.0, about 3.0, about 2.0, about 1.0 or about 0 ppm, and wherein the formulation has a shelf life of at least about 24 months or, more preferably, at least about 36 months under variable storage conditions including, but not limited to, storage at a temperature of about 2° C. to about 8° C., about 8° C. to about 24° C. or about 25° C. to about 30° C.

In a further preferred embodiment, the present invention is a pharmaceutical formulation comprising about 24 mg/mL of pegnivacogin and about 0.1% w/w of methionine, wherein the formulation has a pH of about 7.0 or less, or more particularly about 6.8, and a level of dissolved oxygen that is reduced and more particularly, a level of dissolved oxygen is less than about 5.0, about 4.0, about 3.0, about 2.0, about 1.0 or about 0 ppm, and wherein the formulation has shelf life enhanced over the formulation of Example 1 or oligonucleotide formulations known in the art by about 5, about 10, about 15, about 20, about 25, about 30, about 35, about 40, about 45, about 50, about 55, about 60, about 65, about 70, or about 75% or greater under variable storage conditions including, but not limited to, storage at a temperature of about 2° C. to about 8° C., about 8° C. to about 24° C. or about 25° C. to about 30° C.

In another particular embodiment, the present invention is a pharmaceutical formulation comprising RB571 and from about 0.001 to about 0.1, about 0.005 to about 0.05, about 0.01 to about 0.05 w/w or about 0.001, 0.01, 0.025, 0.05 or 0.1% wt./wt. of at least one antioxidant (e.g., methionine), and wherein the formulation has a pH of about 7.0 or less and/or a level of dissolved oxygen that is ambient or reduced, wherein the reduced dissolved oxygen level is less than about 5.0, about 4.0, about 3.0, about 2.0, about 1.9, about 1.8, about 1.7, about 1.6, about 1.5, about 1.4, about 1.3, about 1.2, about 1.1, about 1.0, about 0.9, about 0.8, about 0.7, about 0.6, about 0.5, about 0.4, about 0.3, about 0.2, about 0.1 or about 0 ppm.

In yet another particular embodiment, the present invention is a pharmaceutical formulation comprising RB571 and from about 0.001 to about 0.1, about 0.005 to about 0.05, about 0.01 to about 0.05 w/w or about 0.001, 0.01, 0.025, 0.05 or 0.1% wt./wt. of at least one antioxidant (e.g., methionine), and wherein the formulation has a pH of about 7.0 or less and a level of dissolved oxygen that is ambient or, more particularly, between about 5 and about 10 ppm.

In an exemplary embodiment, the present invention is a pharmaceutical formulation comprising from about 1 to about 100 mg/mL, or more particularly about 20 to about 30 mg/mL RB571 and from about 0.001 to about 0.1, about 0.005 to about 0.05, about 0.01 to about 0.05 w/w or about 0.001, 0.01, 0.025, 0.05 or 0.1% wt./wt. of methionine, wherein the formulation has a pH of 7.0 or less and a dissolved oxygen level that is ambient or more particularly, about 5 to about 10 ppm.

In another exemplary embodiment, the present invention is a pharmaceutical formulation comprising from about 1 to about 100 mg/mL, or more particularly, about 20 to about 30 mg/mL RB571 and from about 0.001 to about 0.1, about 0.005 to about 0.05, about 0.01 to about 0.05 w/w or about 0.001, 0.01, 0.025, 0.05 or 0.1% wt./wt. of methionine, wherein the formulation has a pH of 7.0 or less and a dissolved oxygen level that is reduced, or more particularly, less than about 5.0, about 4.0, about 3.0, about 2.0, about 2.0, about 1.9, about 1.8, about 1.7, about 1.6, about 1.5, about 1.4, about 1.3, about 1.2, about 1.1, about 1.0, about 0.9, about 0.8, about 0.7, about 0.6, about 0.5, about 0.4, about 0.3, about 0.2, about 0.1 or about 0 ppm.

In a specific embodiment, the present invention is a pharmaceutical formulation comprising about 24 mg/mL of RB571 and from about 0.025 to about 0.1% w/w of methionine, wherein the formulation has a pH of about 7.0 or less and a level of dissolved oxygen that is ambient or reduced, wherein the reduced dissolved oxygen level is less than about 5.0 ppm.

In a preferred embodiment, the present invention is a pharmaceutical formulation In a preferred embodiment, the present invention is a pharmaceutical formulation comprising about 24 mg/mL of RB571 and about 0.1% w/w of methionine, wherein the formulation has a pH of about 7.0 or less, or more particularly, about 6.8, and a level of dissolved oxygen that is ambient or more particularly, between about 5 and about 10 ppm.

In another preferred embodiment, the present invention is a pharmaceutical formulation. In a preferred embodiment, the present invention is a pharmaceutical formulation comprising about 24 mg/mL of RB571 and about 0.1% w/w of methionine, wherein the pH is about 7.0 or less, or more particularly about 6.8, and the level of dissolved oxygen is ambient or between about 5 and about 10 ppm and wherein the formulation has a shelf life of at least about 24 months and preferably, at least about 36 months under variable storage conditions including, for example, temperature of about 2° C. to about 8° C., about 8° C. to about 24° C. or about 25° C. to about 30° C.

In a still further preferred embodiment, the present invention is a pharmaceutical formulation In a preferred embodiment, the present invention is a pharmaceutical formulation comprising about 24 mg/mL of RB571 and about 0.1% w/w of methionine, wherein the pH is about 7.0 or less, or more particularly about 6.8, and the level of dissolved oxygen is ambient or between about 5 and about 10 ppm and wherein the formulation has a shelf life that is enhanced over conjugated oligonucleotide formulations known in the art or the formulation disclosed in Example 1 by about 5, about 10, about 15, about 20, about 25, about 30, about 35, about 40, about 45, about 50, about 55, about 60, about 65, about 70, or about 75% or greater under variable storage conditions including, but not limited to, storage at a temperature of about 2° C. to about 8° C., about 8° C. to about 24° C. or about 25° C. to about 30° C.

In yet another preferred embodiment, the present invention is a pharmaceutical formulation comprising about 24 mg/mL of RB571 and about 0.1% w/w of methionine, wherein the formulation has a pH of about 7.0 or less, or more particularly about 6.8, and a level of dissolved oxygen that is reduced and more particularly, the level of dissolved oxygen less than about 5.0, about 4.0, about 3.0, about 2.0, about 1.0 or about 0 ppm, and wherein the formulation has a shelf life of at least about 24 months and preferably, at least about 36 months under variable storage conditions including, but not limited to, storage at a temperature of about 2° C. to about 8° C., about 8° C. to about 24° C. or about 25° C. to about 30° C.

In a further preferred embodiment, the present invention is a pharmaceutical formulation comprising about 24 mg/mL of RB571 and about 0.1% w/w of methionine, wherein the formulation has a pH of about 7.0 or less, or more particularly about 6.8, and a level of dissolved oxygen that is reduced and more particularly, a level of dissolved oxygen is less than about 5.0, about 4.0, about 3.0, about 2.0, about 1.0 or about 0 ppm, and wherein the formulation has shelf life enhanced over the formulation of Example 1 or oligonucleotide formulations known in the art by about 5, about 10, about 15, about 20, about 25, about 30, about 35, about 40, about 45, about 50, about 55, about 60, about 65, about 70, or about 75% or greater under variable storage conditions including, but not limited to, storage at a temperature of about 2° C. to about 8° C., about 8° C. to about 24° C. or about 25° C. to about 30° C.

### VI. Methods of Preparation

The present invention includes methods for preparing the pharmaceutical formulation of the present invention.

According to one method, an aqueous solvent (or first aqueous solution) is provided. The aqueous solvent may be, for example, an osmotically adjusted buffer. In certain embodiments, the aqueous solvent as provided contains methionine. As discussed further below, the dissolved oxygen content and/or pH of the aqueous solvent may optionally be modified.

According to this first method, the oligonucleotide or conjugated oligonucleotide is provided in the form of a second aqueous solution, in which the oligonucleotide or oligonucleotide conjugate has been previously dissolved. The second aqueous solution may be, for example, a prepared buffer. The concentration of the oligonucleotide or oligonucleotide conjugate in the aqueous solution can be adjusted by adding more solution, as necessary.

According to this first method, the aqueous solvent and the second aqueous solution are mixed to form the aqueous formulation. The first and second solutions can independently comprise other excipients and agents described herein.

According to a second method of preparation, the oligonucleotide or oligonucleotide conjugate is added directly to the aqueous solvent as a solid (i.e., without the prior formation of the second aqueous solution by dissolving the oligonucleotide or oligonucleotide conjugate in a dissolving solvent), thereby forming an aqueous formulation. In a particular embodiment of the second method, the aqueous solvent (first solution) contains methionine. Methionine may be added to the aqueous solvent simultaneously with the oligonucleotide, or may be added to the aqueous formulation, i.e., after the oligonucleotide has been added to the aqueous solvent. Optionally, as described further below, the pH or the dissolved oxygen level of the aqueous solvent and/or the aqueous formulation may be modified.

Optionally, buffers can be added to the first or second solution and/or to the aqueous formulation.

In a first embodiment, the present invention is a method of preparing a pharmaceutical formulation, comprising: (a) providing an aqueous solvent; (b) adding an oligonucleotide or conjugated oligonucleotide (e.g., a pegylated aptamer) to the aqueous solvent to form an aqueous formulation; (c) filtering the aqueous formulation to form a filtered aqueous formulation; (d) filling a storage container with the filtered aqueous formulation; and (e) sealing the storage container, to provide a pharmaceutical formulation having a pH of about 7 or less and an ambient dissolved oxygen level or more particularly, about 5 to about 10 ppm.

In a particular embodiment, the oligonucleotide or oligonucleotide conjugate is added to the aqueous solvent in the form of an aqueous solution, prepared for example by adding the oligonucleotide or oligonucleotide conjugate to a dissolving solvent.

In another particular embodiment, the oligonucleotide or oligonucleotide conjugate is added to the aqueous solvent in the form of a solid.

The pharmaceutical formulation provided by this first embodiment of the method has a desirable shelf life under varied storage conditions. For example, the pharmaceutical formulation prepared by this first embodiment of the method advantageously provides a shelf life of at least 24 months or at least 36 months under varied storage conditions which may include, for example, storage at temperatures of about 2° C. to about 8° C., about 8° C. to about 24° C. or about 25° C. to about 30° C.

Optionally, the method involves one or more additional steps. In one embodiment, the method further involves modifying or reducing the pH of the solvent (including the dissolving solvent), the aqueous formulation or the filtered aqueous formulation. Methods of modifying pH of a solution are known in the art, and appropriate methods will be used to preserve the physiologically compatible characteristics of the solution. The pH of the composition may be modified, for example, using a buffering agent, for example a pharmaceutically acceptable buffering agent. Buffering agents, as indicated above, include, by way of example and without limitation, acetic acid, sodium acetate, adipic acid, benzoic acid, sodium benzoate, citric acid, maleic acid, monobasic sodium phosphate, dibasic sodium phosphate, lactic acid, tartaric acid, glycine, potassium metaphosphate, potassium phosphate, monobasic sodium acetate, sodium bicarbonate, sodium tartrate and sodium citrate anhydrous and dihydrate and others known to those of ordinary skill in the art. Phosphate buffers include, for example, sodium phosphate buffers and sodium phosphate dibasic dihydrate buffers. In a particular embodiment, the pH is modified using HCl, NaOH and/or other acids and bases.

The pH may be modified one or more times during the method. In a particular embodiment, the pH of one or more of the solvents (including the dissolving solvent), the aqueous formulation and/or the filtered aqueous formulation is modified.

In a particular embodiment, the pH is modified before, during and/or after one or more of steps (a)-(d). In a particular embodiment, the pH is modified after the oligonucleotide and buffer are mixed together, prior to any filtration or subsequent processing.

The pH can optionally be measured, before, during, and/or after any pH adjustment step using methods known in the art.

In a second embodiment, the method of the present invention further comprises modifying the dissolved oxygen content of the solvent (including the dissolving solvent), the aqueous formulation or the filtered aqueous formulation or combinations thereof. The dissolved oxygen level can be modified or reduced until a desired level is reached. Methods for modifying dissolved oxygen levels in a solution are known to those of skill in the art. For example, dissolved oxygen may be removed from solutions by an inert gas purging procedure, such as nitrogen gas purging, or by vacuum degassing.

In a particular embodiment, the level of dissolved oxygen is reduced by nitrogen gas purging.

The aqueous formulation and/or the components, e.g., the bulk buffers, may be purged once or more than once. For example, the aqueous solution can be purged just prior to filtration and/or during filling, including continuously during filling. The headspace may also be purged. In a particular embodiment, the dissolved oxygen level is modified or reduced before or during one or more of the steps (a)-(d). In one embodiment, the dissolved oxygen level is controlled such that all the vials in a production batch have substantially the same target dissolved oxygen levels. In one embodiment, the dissolved oxygen of all vials of a production batch is less than about 2 ppm, about 3-5 ppm, about 4-6 ppm, about 6-8 ppm, or about 8-10 ppm. Dissolved oxygen may be reduced or controlled by purging bulk buffers, head space, filtration lines. Dissolved oxygen may also be reduced or controlled by controlling the dissolved oxygen in the vial filling and capping environment.

In a particular embodiment, the dissolved oxygen level of the aqueous solvent is modified after is it provided in (a).

In another particular embodiment, the dissolved oxygen level of the dissolving solvent is modified prior to or after the second aqueous solution is provided to form the aqueous formulation

In yet another particular embodiment, the dissolved oxygen level of the aqueous formulation is modified prior to, during or after filtration.

In a still further embodiment, the dissolved oxygen level of the filtered aqueous formulation is modified prior to, during or after filling. In a particular embodiment, the dissolved oxygen level of the filtered aqueous formulation is modified during filling via overlay and/or by employing a nitrogen sparged isolator for filling.

In a particular embodiment, the aqueous formulation is purged just prior to filtration with nitrogen gas, followed by purging of the headspace.

Dissolved oxygen measurements can be obtained by methods familiar to those of ordinary skill in the art, including the method outlined in Example 2.

According to this second embodiment, the present invention is a method of preparing a pharmaceutical formulation, comprising: (a) providing a aqueous solvent; (b) adding an oligonucleotide or conjugated oligonucleotide (e.g., a pegylated aptamer) to the aqueous solvent to form an aqueous formulation; (c) filtering the aqueous formulation to form a filtered aqueous formulation; (d) filling a storage container with the filtered aqueous formulation; and (e) sealing the storage container to provide a pharmaceutical formulation having level of dissolved oxygen that is reduced and more particularly, a level of dissolved oxygen of less than about 5 ppm, about 4 ppm, about 3 ppm, about 2 ppm, about 1 ppm or about 0 ppm.

Optionally, the second embodiment of the method involves one or more additional steps, including for example, modification of pH as described above with respect to the first embodiment.

The pharmaceutical formulation provided by this second embodiment of the method has a desirable shelf life under varied conditions. For example, the pharmaceutical formulation prepared by this first embodiment of the method advantageously provides a shelf life of at least about 24 months or, more preferably, at least about 36 months under varied storied conditions which may include, for example, storage at temperatures of about 2° C. to about 8° C., about 8° C. to about 24° C. or about 25° C. to about 30° C.

In a third embodiment, the present invention is a method of preparing the pharmaceutical formulation, comprising: (a) providing an aqueous solvent comprising at least one antioxidant; (b) adding an oligonucleotide or oligonucleotide conjugate (e.g., a pegylated oligonucleotide such as a pegylated aptamer) to the aqueous solvent to form an aqueous formulation; (c) filtering the aqueous formulation to form a filtered aqueous formulation; (d) filling a storage container with the filtered aqueous formulation; and (e) sealing the storage container.

In one embodiment, the antioxidant is a reducing agent.

In a particular embodiment, the antioxidant is methionine.

In a fourth embodiment, the present invention is a method of preparing the pharmaceutical formulation, comprising: (a) providing an aqueous solvent; (b) adding an oligonucleotide or conjugated oligonucleotide and at least one antioxidant to the aqueous solvent to form an aqueous formulation; (c) filtering the aqueous formulation to form a filtered aqueous formulation; (d) filling a storage container with the filtered aqueous formulation; and (e) sealing the storage container, to provide a pharmaceutical formulation having a shelf life of at least about 24 months, or, more preferably, at least about 36 months at about 2° C. to about 8° C.

In a particular embodiment, the antioxidant is a reducing agent.

In another particular embodiment, the antioxidant is methionine.

In a particular embodiment, the oligonucleotide or conjugated oligonucleotide and the at least one antioxidant are added to the aqueous solvent simultaneously.

In another particular embodiment, the at least one antioxidant is added to the aqueous formulation, i.e., after the oligonucleotide or conjugated oligonucleotide has been added to the aqueous solvent.

The pharmaceutical formulation provided by this third and fourth embodiment of the method has a desirable shelf life under varied storage conditions. For example, the pharmaceutical formulation prepared by this first embodiment of the method advantageously provides a shelf life of at least about 24 months or, more preferably, at least about 36 months under varied storied conditions which may include, for example, storage at temperatures of about 2° C. to about 8° C., about 8° C. to about 24° C. or about 25° C. to about 30° C.

In a fifth embodiment, the present invention is a method of preparing the pharmaceutical formulation, comprising: (a) providing an aqueous solvent and at least one antioxidant (e.g., methiononine); (b) adding an oligonucleotide or conjugated oligonucleotide to the aqueous solvent to form an aqueous formulation; (c) filtering the aqueous formulation to form a filtered aqueous formulation; (d) filling a storage container with the filtered aqueous formulation; and (e) sealing the storage container, to provide a pharmaceutical formulation having pH of about 7 or less and/or a dissolved oxygen content that is reduced.

In particular embodiment, the dissolved oxygen content is reduced one or more times by sparging or other suitable method. Methods of sparging the composition are known in the art of pharmaceutical formulation and the formulations described herein may be sparged with an acceptable medical gas such as, but not limited to, nitrogen. Other acceptable medical gases are known to those skilled in the art. The formulations may be sparged at appropriate times during the manufacturing process such as sparging the buffer during manufacture, or sparging the liquid before filling and sealing vials for storage and/or shipping. The formulation may be sparged continuously throughout the manufacturing process or intermittently as needed to keep oxygen levels at appropriate levels for production and storage. Sparging may also be used for overlay or blanket during filling or in a nitrogen-filled isolator encasing the filling line. In one embodiment, sparging is used to maintain a consistent level of dissolved oxygen in vials throughout the filling step. In another embodiment, vials are sealed sufficiently to maintain substantially the same dissolved oxygen level until the vial is unsealed.

Optionally, the method further comprises adjusting the pH one or more times, as described above with respect to the first and second embodiments.

The pharmaceutical formulation provided by this sixth embodiment of the method has a desirable shelf life under varied storage conditions. For example, the pharmaceutical formulation prepared by this first embodiment of the method advantageously provides a shelf life of at least 24 months or at least 36 months under varied storied conditions which may include, for example, storage at temperatures of about 2° C. to about 8° C., about 8° C. to about 24° C. or about 25° C. to about 30° C.

The oligonucleotide or oligonucleotide conjugate of the method may vary. In one embodiment of the method, the oligonucleotide conjugate is a pegylated aptamer.

In an exemplary embodiment, the present invention is a method of preparing the pharmaceutical formulation, comprising: (a) providing an aqueous solvent; (b) adding pegylated apatmer and at least one antioxidant to the aqueous solvent to form an aqueous formulation; (c) filtering the aqueous formulation to form a filtered aqueous formulation; (d) filling a storage container with the filtered aqueous formulation; and (e) sealing the storage container, to provide a pharmaceutical formulation having a pH of about 7 or less and a dissolved oxygen content that is ambient or reduced (e.g., less than about 5 ppm), wherein the formulation has a shelf life of at least about 24 or at least about 36 months under varied storage conditions including, for example, storage at about 2° C. to about 8° C., about 8° C. to about 24° C. or about 24° C. to about 30° C.

In another exemplary embodiment, the present invention is a method of preparing the pharmaceutical formulation, comprising: (a) providing an aqueous solvent comprising at least one antioxidant; (b) adding an oligonucleotide or conjugated oligonucleotide and at least one antioxidant to the aqueous solvent to form an aqueous formulation; (c) filtering the aqueous formulation to form a filtered aqueous formulation; (d) filling a storage container with the filtered aqueous formulation; and (e) sealing the storage container, to provide a pharmaceutical formulation having a pH of about 7 or less and a dissolved oxygen content that is ambient or reduced (e.g., less than about 5 ppm), wherein the formulation has a shelf life of at least about 24 or, more preferably, at least about 36 months under varied storage conditions including, for example, storage at about 2° C. to about 8° C., about 8 to about 24° C. or about 24° C. to about 30° C.

In a specific embodiment, the oligonucleotide conjugate is pegnivacogin.

In an exemplary embodiment, the present invention is a method of preparing the pharmaceutical formulation, comprising: (a) providing an aqueous solvent; (b) adding pegnivacogin and at least one antioxidant (e.g., methionine) to the aqueous solvent to form an aqueous formulation, wherein pegnivacogin can be added to the aqueous solvent as a solid or in the form of an aqueous solution and wherein the antioxidant can be added before or after the pegnivacogin; (c) filtering the aqueous formulation to form a filtered aqueous formulation; (d) filling a storage container with the filtered aqueous formulation; and (e) sealing the storage container, to provide a pharmaceutical formulation having a pH of about 7 or less and a dissolved oxygen content that is ambient or reduced (e.g., less than about 5 ppm), wherein the formulation has a shelf life of at least about 24 or, more preferably, at least about 36 months under varied storage conditions including, for example, storage at about 2° C. to about 8° C., about 8° C. to about 24° C. or about 24° C. to about 30° C.

In another exemplary embodiment, the present invention is a method of preparing the pharmaceutical formulation, comprising: (a) providing an aqueous solvent comprising at least one antioxidant (e.g., methionine); (b) adding pegnivacogin to the aqueous solvent to form an aqueous formulation wherein pegnivacogin can be added to the aqueous solvent as a solid or in the form of an aqueous solution and wherein the antioxidant can be added before or after the pegnivacogin; (c) filtering the aqueous formulation to form a filtered aqueous formulation; (d) filling a storage container with the filtered aqueous formulation; and (e) sealing the storage container, to provide a pharmaceutical formulation having a pH of about 7 or less and a dissolved oxygen content that is ambient or reduced (e.g., less than about 5 ppm), wherein the formulation has a shelf life of at least about 24 or, more preferably, at least about 36 months under varied storage conditions including, for example, storage at about 2° C. to about 8° C., about 8° C. to about 24° C. or about 24° C. to about 30° C.

In a specific embodiment, the oligonucleotide conjugate is RB571.

In an exemplary embodiment, the present invention is a method of preparing the pharmaceutical formulation, comprising: (a) providing an aqueous solvent; (b) adding RB571 and at least one antioxidant (e.g., methionine) to the aqueous solvent to form an aqueous formulation, wherein RB571 can be added to the aqueous solvent as a solid or in the form of an aqueous solution and wherein the antioxidant can be added before or after the RB571; (c) filtering the aqueous formulation to form a filtered aqueous formulation; (d) filling a storage container with the filtered aqueous formulation; and (e) sealing the storage container, to provide a pharmaceutical formulation having a pH of about 7 or less and a dissolved oxygen content that is ambient or reduced (e.g., less than about 5 ppm), wherein the formulation has a shelf life of at least about 24 or, more preferably, at least about 36 months under varied storage conditions including, for example, storage at about 2° C. to about 8° C., about 8° C. to about 24° C. or about 24° C. to about 30° C.

In another exemplary embodiment, the present invention is a method of preparing the pharmaceutical formulation, comprising: (a) providing an aqueous solvent comprising at least one antioxidant (e.g., methionine); (b) adding RB571 to the aqueous solvent to form an aqueous formulation wherein RB571 can be added to the aqueous solvent as a solid or in the form of an aqueous solution and wherein the antioxidant can be added before or after the RB571; (c) filtering the aqueous formulation to form a filtered aqueous formulation; (d) filling a storage container with the filtered aqueous formulation; and (e) sealing the storage container, to provide a pharmaceutical formulation having a pH of about 7 or less and a dissolved oxygen content that is ambient or reduced (e.g., less than about 5 ppm), wherein the formulation has a shelf life of at least about 24 or, more preferably, at least about 36 months under varied storage conditions including, for example, storage at about 2° C. to about 8° C., about 8° C. to about 24° C. or about 24° C. to about 30° C.

The amount of the oligonucleotide or oligonucleotide conjugate may also vary. In one embodiment of the method, the concentration of the oligonucleotide or oligonucleotide conjugate is from about 1 to about 100, about 20 to about 30 or about 24 mg/mL.

If present, the amount of the antioxidant may also vary. In one embodiment of the method, the amount of antioxidant is from about 0.001 to about 1.0% w/w, or about 0.01 to about 0.5% w/w. In a specific embodiment, the antioxidant is methionine and it is present from about 0.001 to about 1.0%% w/w, about 0.01 to about 0.5% w/w, about 0.5 to about 0.2% w/w, or about 0.1% w/w.

One or more additional steps may take place prior to filling the storage container with the aqueous formulation. For example, the formulation may be brought to isotonicity using an appropriate amount of an isotonicity agent. Also prior to filling, the pH and/or oligonucleotide content may be determined. The pH may be determined as described above. The oligonucleotide content can be determined according to methods known to those of skill in the art, for example as described in Example 1 with respect to pegnivacogin content.

The pharmaceutical formulation of the present invention can be stored in containers commonly used in the pharmaceutical industry, including containers typically used for parenteral formulations, which can include plastic containers or glass containers such as standard USP/EP

Type I borosilicate glass containers. For example, the container used can be a syringe or vial.

In one embodiment, the vial is sealed under nitrogen purge using a nitrogen purge system. A nitrogen purge system can greatly benefit a packaging system in several different ways. One way is by removing oxygen from the headspace of a container just before the container is capped or otherwise sealed. By replacing the oxygen in the headspace with a nitrogen gas, the product being packaged gains several advantages. As bottles pass under the nitrogen purge system, a blast of nitrogen gas is shot into the container opening. The containers then immediately enter the capping machine to be sealed, the headspace full of nitrogen rather than oxygen. In another embodiment, the containers are filled in an isolator containing nitrogen to fill the headspace full of nitrogen rather than oxygen.

In one embodiment, the storage container is a vial having a stopper and overseal. In a particular embodiment, the vial is a 6 mL glass vial. The stopper may be a stopper compatible with the oligonucleotide as well as the other components of the formulation, for example, a rubber stopper. Non-limiting examples include pre-sterilized, siliconized 4432/50 gray butyl rubber stoppers and Teflon® coated 4432/50 gray butyl rubber stoppers. The overseal may vary and include, for example, a Standard Flip-Off® overseal constructed of an aluminum cap and a blue plastic Flip-Off® tamper evident overseal.

In one embodiment, the present invention is an article of manufacture comprising a container containing the pharmaceutical formulation of the invention. Suitable containers are discussed above.

The aqueous formulation of the invention can be provided in a kit.

### VII. Methods of Administration and Use

The pharmaceutical formulations of the present invention are administered to a host in need thereof. The host may be, for example, a mammal and more specifically, a human. Optionally, the pharmaceutical formulation may be diluted prior to use (e.g., using normal saline or sterile water for injection).

In one embodiment, the present invention is a method for treating a host in need thereof, comprising administering to the host a pharmaceutical formulation comprising an oligonucleotide or an oligonucleotide conjugate (e.g., a pegylated aptamer) and at least one antioxidant, including but not limited to one or more reducing agents such as methionine.

In another embodiment, the present invention is a method for treating a host in need thereof, comprising administering to the host a pharmaceutical formulation comprising an oligonucleotide or an oligonucleotide conjugate (e.g., a pegylated aptamer), wherein the formulation has a pH of about 7 or less.

In a particular embodiment, the present invention is a method for treating a host in need thereof, comprising administering to the host a pharmaceutical formulation comprising an oligonucleotide or oligonucleotide conjugate (e.g., a pegylated aptamer), wherein the formulation has a pH of about 6.8.

In a further embodiment, the present invention is a method for treating a host in need thereof, comprising administering to the host a pharmaceutical formulation comprising an oligonucleotide or oligonucleotide conjugate, wherein the formulation has dissolved oxygen level that is ambient or reduced, wherein the reduced dissolved oxygen level is less than about 5 ppm

In a still further embodiment, the present invention is a method for treating a host in need thereof, comprising administering to the host a pharmaceutical formulation comprising an oligonucleotide or an oligonucleotide conjugate, wherein the formulation has a pH of about 7 or less and a dissolved oxygen level that is ambient or reduced, wherein the reduced dissolved oxygen less is less than about 5 ppm.

In a still further embodiment, the present invention is a method for treating a host in need thereof, comprising administering to the host a pharmaceutical formulation comprising an oligonucleotide or oligonucleotide conjugate and at least one antioxidant (e.g., a reducing agent such as methionine), wherein the formulation has a pH of about 7 or less or more preferably, about 6.8.

In another embodiment, the present invention is a method for treating a host in need thereof, comprising administering to the host a pharmaceutical formulation comprising an oligonucleotide or oligonucleotide conjugate and at least one antioxidant (e.g., a reducing agent such as methionine), wherein the formulation has dissolved oxygen level that is ambient or reduced, wherein the reduced dissolved oxygen level is less than about 5 ppm.

In yet another embodiment, the present invention is a method for treating a host in need thereof, comprising administering to the host a pharmaceutical formulation comprising an oligonucleotide or oligonucleotide conjugate and at least one antioxidant (e.g., a reducing agent such as methionine), wherein the formulation has a pH of about 7 or less, and more preferably about 6.8, and a dissolved oxygen level that is ambient or between about 5 and about 10 ppm.

In a preferred embodiment, the present invention is a method for treating a host in need thereof, comprising administering to the host a pharmaceutical formulation comprising pegnivacogin and methionine, wherein the formulation has a pH of about 7 or less, and more preferably about 6.8, and a dissolved oxygen level that is ambient or between about 5 and about 10 ppm.

In a preferred embodiment, the present invention is a method for treating a host in need thereof, comprising administering to the host a pharmaceutical formulation comprising RB571 and methionine, wherein the formulation has a pH of about 7 or less, and more preferably about 6.8, and a dissolved oxygen level that is ambient or between about 5 and about 10 ppm.

Optionally, a modulator is administered to the host subsequent to the administration of the pharmaceutical formulation of the present invention.

The host may be in need of treatment as the result of a disease or disorder impacting the host. The disease or disorder may be infectious or non-infectious in nature. Representative non-infectious diseases and disorders include cardiovascular diseases, cancer, inflammatory diseases and neurological disorders.

In one embodiment, the host is in need of anticoagulation. In a particular embodiment, the host is suffering from an acute coronary syndrome, such as unstable angina or myocardial infarction. In another particular embodiment, the host is undergoing a coronary revascularization procedure. The procedure may be, for example, coronary artery bypass graft (CABG) surgery or percutaneous coronary interventions (PCI).

Specially, the formulation of the present invention can be used as an antidote-reversible anticoagulant in a host undergoing CABG surgery or PCI, as an antidote-reversible anticoagulant for use in patients suffering from acute coronary syndromes, and as an anticoagulant for other indications in which it would be advantageous to employ an antidote-reversible agent for anticoagulant or antithrombotic therapy. Disorders and procedures for which the pharmaceutical formulations of the invention may be used include, but are not limited to, peripheral vessel graft procedures, including those associated with the iliac, carotid, brachial, aorta, renal, mesenteric, femoral, popliteal, tibial, and peritoneal vessels; the prevention or treatment of deep vein thrombosis; the prevention or treatment of pulmonary embolism following orthopedic surgery or in patients with cancer; the prevention of atrial fibrillation; the prevention of thrombotic stroke; and in indications requiring extracorporeal circulation of blood including but not limited to hemodialysis and extracorporeal membrane oxygenation. Additional examples of potential disorders and procedures for which the formulations of the present invention can be used include, but are not limited to, patients undergoing intracardiac surgery on cardiopulmonary bypass; patients with intracardiac clot formation or peripheral embolization; and patients that are in other hypercoagulable states. The formulations of the present invention may also be useful for prevention of DVT and pulmonary embolization on immobilized patients and for maintenance of potency of indwelling intravenous catheters and arterial or in venous lines.

In one embodiment, the host is suffering from a structural heart disease like valvular heart disease, and the present invention can be used as an antidote-reversible anticoagulant in a host undergoing transcatheter aortic valve replacement or implantation (TAVR/TAVI).

When the oligonucleotide conjugate is pegnivacogin, the range of doses will be dependent upon the indication and will be an effective amount of RB006 sufficient to produce a desired physiological effect such as anticoagulation. For example, the RB006 dose can be in humans from about 0.1 mg/kg to about 10 mg/kg. In certain indications, the dose range will be about from 0.5 mg/kg to about 9 mg/kg, from about 0.75 mg/kg to about 8 mg/kg, from about 1 mg/kg to about 7 mg/kg, from about 1.5 mg/kg to about 6.0 mg/kg, from about 2.0 mg/kg to about 5.0 mg/kg, from about 2.5 mg/kg to about 4.0 mg/kg. In a particular embodiment, the RB006 dose is about 1.0 mg/kg. In certain indications, the drug component will be administered at a dose necessary to maintain the patency of the procedure. In certain indications, RB006 will be administered alone, without subsequent administration of a neutralizing antidote.

The corresponding dose of the antidote component of REG1, RB007, is a therapeutically effective amount required to neutralize or partially neutralize RB006 and is dependent upon the amount of RB006 administered. Generally, a therapeutically effective amount is an amount of the antidote sufficient to produce a measurable modulation of the effects of the nucleic acid ligand, including but not limited to a coagulation-modulating amount or an inflammation-modulating amount. The antidote dose can range, in a antidote:drug weight ratio (mgs of antidote:mgs of drug), from about 0.1:1 to about 20:1, from about 0.25:1 to about 15:1, from about 0.5:1 to about 12:1, from about 0.75 to about 10:1, from about 1:1 to about 9:1, from about 1.5:1 to about 8:1, from about 2:1 to about 7.5:1, from about 2.5:1 to about 6:1, from about 3:1 to about 5:1. In a particular embodiment, the antidote dose is about 0.5:1.0.

In one embodiment, the present invention includes a method of administration of an aptamer anticoagulant system comprising: 1) measuring the body mass index (BMI) of a host; 2) identifying a desired pharmacodynamic response; and 3) administering to the host a dose of a pharmaceutical formulation of a pegylated aptamer to achieve a desired pharmacodynamic response based on a comparison of the dose per BMI to pharmacodynamic response. In certain embodiments, an antidote to the aptamer is subsequently administered to the host where the dose of antidote is provided based on a ratio with the dose of aptamer previously administered adjusted for a desired reduction in aptamer activity. In certain instances, this dose of antidote is adjusted based on the time after administration of the aptamer. In certain instances, the ratio of antidote to aptamer is halved if the aptamer has been administered more than 24 hours previously.

In certain embodiments, a maximal level of anti-coagulation effect is desired. In these instances, an aptamer can be provided at a level of 4 mg/BMI or greater. In other instances, a level of anticoagulation of about 75% maximal is desired. In those instances, a dose of about between 0.75.0-1.5 mg/BMI is provided to the host. In other instances, a level of anticoagulation of about 50% maximal is desired. In these instances, a dose of about 0.25-0.5 mg/BMI is provided.

In certain general embodiments, the dosage of anticoagulant used is between 0.1 and 10 mg/BMI. In another embodiment, the dosage is between 0.2 and 8 mg/BMI, or between 0.2 and 6 mg/BMI, between 0.2 and 5 mg/BMI, between 0.2 and 4 mg/BMI, between 0.2 and 3 mg/BMI, between 0.2 and 2 mg/BMI, or between 0.2 and 1 mg/BMI. In some embodiments, the dose of anticoagulant is about 0.1 mg/BMI, or about 0.2 mg/BMI, or about 0.5 mg/BMI, or about 0.75 mg/BMI, or about 1 mg/BMI, or about 2 mg/BMI, or about 3 mg/BMI, or about 4 mg/BMI, or about 5 mg/BMI, or about 6 mg/BMI, or about 7 mg/BMI, or about 8 mg/BMI, or about 9 mg/BMI, or about 10 mg/BMI.

In another embodiment, the present invention provides an improved method of administration of an aptamer anticoagulant system comprising: 1) measuring the weight of a host; 2) identifying a desired pharmacodynamic response; and 3) administering to the host a dose of an aptamer anticoagulant to achieve a desired pharmacodynamic response based on a comparison of the dose per kilogram of host weight to pharmacodynamic response. In certain embodiments, an antidote to the aptamer is subsequently administered to the host where the dose of antidote is provided based on a ratio with the dose of aptamer previously administered adjusted for a desired reduction in aptamer activity. In certain instances, this dose of antidote is adjusted based on the time after administration of the aptamer. In certain instances, the ratio of antidote to aptamer is halved if the aptamer has been administered more than 24 hours previously.

In certain embodiments, a maximal level of anti-coagulation effect is desired. In these instances, an aptamer can be provided at a level of ≧0.75 mg/kg or greater. In other instances, a level of anticoagulation of about 75% maximal is desired. In those instances, a dose of between 0.4 and 0.6 mg/kg is provided to the host. In other instances, a level of anticoagulation of about 50% maximal is desired. In these instances, a dose of about 0.2-0.3 mg/kg is provided.

In certain general embodiments, the dose used is between 0.1 and 2 mg/kg, between 0.1 and 1.8 mg/kg, between 0.1 and 1.6 mg/kg, between 0.1 and 1.5 mg/kg, between 0.1 and 1.4 mg/kg, between 0.1 and 1.3 mg/kg, between 0.1 and 1.2 mg/kg, between 0.1 and 1.1 mg/kg, between 0.1 and 1.0 mg/kg, between 0.1 and 0.9 mg/kg, between 0.1 and 0.8 mg/kg, between 0.1 and 0.7 mg/kg, between 0.1 and 0.6 mg/kg, between 0.1 and 0.5 mg/kg, between 0.1 and 0.4 mg/kg, between 0.1 and 0.3 mg/kg, or between 0.1 and 0.2 mg/kg. In other embodiments, the dose is between 1 and 20 mg/kg, between 1 and 18 mg/kg, between 1 and 15 mg/kg, between 2 and 15 mg/kg, between 3 and 15 mg/kg, between 4 and 15 mg/kg, between 5 and 20 mg/kg, between 5 and 15 mg/kg, or between 1 and 10 mg/kg, or between 5 and 10 mg/kg, or is about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, or about 10 mg/kg. In a principle embodiment, the aptamer anticoagulant system is the REG1 system, which comprises an aptamer anticoagulant and an oligonucleotide antidote. In certain, non-limiting embodiments, the aptamer is RB006 (SEQ ID NO: 1) and the antidote is RB007 (SEQ ID NO: 2). In one embodiment, the pharmacodynamic response is measured in coagulation assays such as the aPTT (plasma or whole blood) or the Activated Clotting Time (ACT), and can be reported as the absolute value, the effect relative to or normalized to baseline measures, the percent effect, percent change, time weighted average or area under the curve over a defined time period.

In a preferred embodiment, the present invention is a method for treating a host in need thereof, comprising administering to the host a pharmaceutical formulation comprising pegnivacogin and methionine, wherein the formulation has a pH of about 7 or less, and more preferably about 6.8, and a dissolved oxygen level that is ambient or between about 5 and about 10 ppm, wherein the dose of pegnivacogin is 1 mg/kg.

The level of pharmacodynamic response can be at any level desired for a particular application. For example, in certain instances when a patient is at low risk for a thrombotic event, a low level of response may be desired. In particular instances, it may not be desirable to maximize clotting factor inhibition, and in particular FIX or FIXa inhibition by using a saturating amount of anticoagulant, particularly an aptamer to FIXa such as RB006. In other instances, when a patient is at a high risk for a thrombotic event or is having a thrombotic episode, a high level of response may be desired. In such instances, it may be desirable to maximize clotting factor inhibition, and in particular, FIX or FIXa inhibition by using a saturating amount of anticoagulant, particularly an aptamer to FIXa such as RB006.

Preferred modes of administration of the materials of the present invention to a mammalian host are parenteral, intravenous or subcutaneous.

In one embodiment, the oligonucleotide therapeutic is provided by intravenous delivery. In a particular embodiment, the oligonucleotide therapeutic is provided by bolus intravenous injection.

The procedures described herein allow for a step wise delivery of both anticoagulant and antidote to allow titration of either or both compounds to a desired level of target inhibition and reversal.

In another embodiment, formulations of GPVI aptamer ligands such as RB571 and the corresponding antidote RB515 can be used to treat a variety of platelet-mediated disorders such as those platelet-mediated disorders commonly associated with diabetes. In one embodiment, a method of treating a subject suffering from diabetes is provided, comprising administration of a GPVI ligand. Treating high-risk diabetic patients with GPVI ligands can reduce or prevent diabetes-associated disorders in these patients. These disorders include, but are not limited to, diabetic retinopathy, diabetic vasculopathy, atherosclerosis, ischemic stroke, and chronic renal failure. Treatment of diabetics with GPVI ligands can also reduce or inhibit microthrombus formation in these patients.

Also provided is a method for treating subjects suffering from platelet-mediated inflammatory disorders such as rheumatoid arthritis (RA) or other inflammatory arthritis disorders. Recent studies have shown that people who suffer from RA and other forms of inflammatory arthritis have increased levels of platelet microparticles in their joint fluids (Boilard et al., Science, 2010, 327:580-583). Platelet microparticles are pro-inflammatory and elicit an inflammatory response from surrounding cells (e.g. synovial fibroblasts). For example, binding of collagen type IV to GPVI results in the release of IL-1 and IL-8.

GPVI ligand formulations can also be used to treat subjects suffering from scleroderma, or systemic sclerosis. Scleroderma appears to occur as an autoimmune response that produces swelling (inflammation) in the muscles and joints, associated with overproduction of collagen. Microvascular injury is one of the major pathogenic processes involved in systemic sclerosis or scleroderma. Interaction of the platelet type I and III collagen receptor (GPVI) with its respective ligand in the exposed subendothelial stroma as a result of ongoing microvascular injury in systemic sclerosis patients results in platelet activation and aggregation with the release of pro-inflammatory mediators, which contribute to vascular damage and inflammation (Chiang et al., Thrombosis, 2006, 117:299-306). In systemic sclerosis, vascular lesions are characterized by an arteriolar-capillary perivasculitis with mononuclear cell infiltration that leads to arterial intimal proliferation and obliteration of arterioles and capillaries with attrition of endothelial cells and basal lamina. A recurring pattern of injury to the endothelial cells or basal lamina, or both, is characteristic of systemic sclerosis. Additionally, these events are driven by the overproduction and accumulation of collagen in body tissues, leading to extensive hardening and scarring of tissues throughout the body. Accordingly, the use of GPVI ligands can provide therapeutic relief from a disease such as scleroderma or systemic sclerosis which is associated with increased levels of collagen and platelet-mediated microvascular injury. A method of treating a subject suffering from scleroderma by administering a therapeutically effective amount of a GPVI ligand is provided herein.

The GPVI ligand formulations disclosed herein can also be used to treat subjects diagnosed with cancer. Recent studies suggest that GPVI mediates tumor metastasis (see, e.g., Jain et al., J. Thromb. Haemostasis, 2009, 7:1713-1717). Using an in vivo experimental metastasis assay, Jain et al., show that GPVI knockout mice exhibited a significant decrease in tumor metastasis as compared to wildtype control mice. Accordingly, in one embodiment, a method for inhibiting, reducing or preventing metastasis in a subject diagnosed as having a primary cancerous tumor is provided, wherein the subject is administered a therapeutically effective amount of a GPVI ligand.

The GPVI nucleic acid ligand formulations are also provided as modulatable anti-platelet agents for use in disorders or treatment regimes requiring anti-platelet therapy. In certain embodiments, the treatment is a surgical intervention. The methods of use can include administering the GPVI nucleic acid ligand to a host in need thereof, wherein the host is suffering from, or at risk of suffering from, an occlusive thrombotic disease or disorder of the coronary, cerebral or peripheral vascular system.

In one embodiment, the GPVI ligand inhibits initiation of platelet activation. In other embodiments, the GPVI ligand inhibits platelet activation and the resultant platelet pro-inflammatory response. In other embodiments, the GPVI ligand inhibits platelet adhesion. In other embodiments, the GPVI ligand inhibits platelet aggregation. In yet a further embodiment, the GPVI ligand inhibits thrombin generation.

In one embodiment, the host has or is at risk of having an occlusive thrombotic disease of the coronary, cerebral and peripheral vascular systems. In certain other embodiments, the host is preparing to undergo or undergoing a surgical intervention, or has undergone a surgical intervention that puts the host at risk of an occlusive thrombotic event. In other embodiments, the host has received a vessel graft to enable hemodialysis, which is at risk of occluding due to interactions between the vessel and platelets.

In certain embodiments a method of treating or preventing formation of a vascular event, in particular a thrombotic or thromboembolitic event is provided including administering a GPVI nucleic acid ligand of the invention to a host in need thereof.

In one embodiment, the GPVI nucleic acid ligand formulation is provided for extended periods of time. In this instance, a GPVI ligand modulator may only be used in emergency situations, for example, if treatment leads to hemorrhage, including intracranial or gastrointestinal hemorrhage. In another embodiment, the modulator is administered when emergency surgery is required for patients who have received GPVI nucleic acid ligand treatment. In another embodiment, the modulator is administered to control the concentration of the GPVI nucleic acid ligand and thereby the duration and intensity of treatment. In another embodiment, the GPVI nucleic acid ligand formulation is provided as a platelet anesthetic during a cardiopulmonary bypass procedure. In another embodiment, the GPVI nucleic acid ligand formulation is administered to provide a period of transition off of or on to oral anti-platelet medications, and the modulator is used to reverse the GPVI nucleic acid ligand once therapeutic levels of the oral anti-platelet agent are established.

In another embodiment, the oligonucleotide therapeutic is provided by intravenous delivery and the modulator is provided by intravenous delivery. In a particular embodiment, both the oligonucleotide therapeutic and the modulator is provided by bolus intravenous injection.

In another embodiment, the oligonucleotide therapeutic and the modulator are provided by different administration modes. In a particular embodiment, the oligonucleotide therapeutic is administered subcutaneously and the modulator is administered intravenously.

The ratio of antidote to aptamer is adjusted based on the desired level of inhibition of the aptamer. It was found that the antidote dose need only correlate to the dose of aptamer, and need not be additionally adjusted based on factors relating to the host. In one embodiment, the ratio of aptamer to antidote is 1:1. In other embodiments, the ratio of aptamer to antidote is greater than about 1:1 such as about 2:1, about 3:1, about 4:1, about 5:1, about 6:1, about 7:1, about 8:1, about 9:1, about 10:1 or more. These ratios can also be calculated based on antidote to aptamer ratio, which can, for example, be less than about 1:1 such as about 0.9:1 or about 0.9:1, 0.8:1 or about 0.8:1, 0.7:1 or about 0.7:1, 0.6:1 or about 0.6:1, 0.5:1 or about 0.5:1, 0.45:1 or about 0.45:1, 0.4:1 or about 0.4:1, 0.35:1 or about 0.35:1, 0.3:1 or about 0.3:1, 0.25:1 or about 0.25:1, about 0.2:1 or about 0.2:1, 0.15:1 or about 0.15:1, 0.1:1 or about 0.1:1 or less than 0.1:1 such as about 0.005:1 or less. In some embodiments, the ratio is between about 0.5:1 and about 0.1:1, or between about 0.5:1 and about 0.2:1, or between about 0.5:1 and about 0.3:1. In other embodiments, the ratio is between about 1:1 and about 5:1, or between about 1:1 and about 10:1, or between about 1:1 and about 20:1.

In some embodiments, only a partial reversal of aptamer activity occurs. For example, in some embodiments, aptamer activity is reversed by about 90%, or less than about 90% such as about 80%, about 70%, about 60%, about 50%, about 40%, about 30%, about 20%, about 10% or less. The ratio of antidote to aptamer can be calculated either by comparing weight to weight or on a molar basis.

In a preferred embodiment, the present invention is a method for treating a host in need of anticoagulation, comprising administering to the host a pharmaceutical formulation comprising pegnivacogin and methionine, wherein the formulation has a pH of about 7 or less, and more preferably about 6.8, and a dissolved oxygen level that is ambient or between about 5 and about 10 ppm.

All patents and patent publications referred to herein are hereby incorporated by reference.

The following examples are used to further illustrate the present invention and should not be considered limiting.

### EXAMPLES

### Example 1

### Formulation of Injection Pegnivacogin Drug Substance

Pegnivacogin (RB006) for injection (“pegnivacogin injection” or “injection pegnivacogin”) was aseptically manufactured under GMP conditions as follows. Formulation buffer of pH of 7.4 was prepared with sodium phosphate buffer and brought to isotonicity using an appropriate amount of sodium chloride to yield a target osmolality of 290±30 mOsm/Kg. The formulation was then filtered through a 0.22 um filter. RB006 was added to the formulation buffer to achieve a nominal concentration of 21 mg/mL. During the formulation of the bulk drug product solution, the solution was purged with N₂ gas. After the bulk solution was prepared and assay values met in-process specifications for pH, osmolality and RB006 content, the product was aseptically filtered and filled into pre-sterilized components

The vials, stoppers and overseals for the container closure were selected from standard commercially available components based upon the neutral pH of pegnivacogin injection. The glass vials utilized for all batches were treated Type I borosilicate glass, and a 6 mL vial was made to reduce head space. The closure was a pre-sterilized, siliconized 4432/50 gray butyl rubber stopper. Standard Flip-Off® overseals constructed of an aluminum cap and a blue plastic Flip-Of® tamper evident overseal were utilized.

An exemplary formulation of pegnivacogen is provided below:

| Pegnivacogin Injection, 24 mg/mL, | | | | ~Osmolality |
|---|---|---|---|---|
| pH 6.8, Methionine = 0.10% | mg/mL | | mM | (mOsmol/kg) |
| Pegnivacogin | 24 | | 0.47 | 29.7 |
| Monobasic Sodium Phosphate, | 4.65 | | 33.70 | 67.39 |
| Monohydrate | | | | |
| Dibasic Sodium Phosphate, | 8.67 | | 32.35 | 64.70 |
| Heptahydrate | | | | |
| Methionine | 1.00 | | 6.70 | 6.70 |
| Sodium Chloride | 3.60 | | 61.64 | 123.29 |
| Water for Injection, USP | q.s. to 1 | mL | | |
| Total | | | | 291.79 |

In total, seven batches were made and stability data was gathered at the storage condition of 2-8° C. for up to 36 months and for accelerated storage at 25° C. for at least 6 months. Dissolved oxygen (DO₂) levels measured in the bulk drug product solution (measured as described in Example 2) fell within the range of 1.7 to 4.7 ppm as measured in the bulk drug product solution.

### Example 2

### Analytical Methods

### A. UV Assay

Assay measurements during formulation development were performed using an Agilent UV spectrophotometer to measure the absorbance at 259 nm of diluted (0.1 mg/mL) formulation samples. A previously determined extinction coefficient and purity correction for the API was used to convert absorbance at 259 nm to RB006 concentration (mg/mL).

### B. Anion Exchange (AX-HPLC) Purity

Anion Exchange (AX-HPLC) of pegnivacogin was performed using a Dionex DNAPac PA-100 (4.0×250 mm) column using an increasing salt gradient of NaCl in Tris acetonitrile buffer. UV detection is achieved at 259 nm using an injection volume of 20 μL, flow rate of 1.2 mL/min and a run time of 20 minutes.

The method is specific for pegnivacogin and can discriminate between pegnivacogin, closely related substances, unknown impurities and degradants. The method has been shown to resolve several synthetically prepared N+1 and N−1 derivatives, degradation products and pegylated pegnivacogin species with molecular weights of 20 kDa, 30 kDa, and 60 kDa.

Pegnivacogin API Lot, R01AZ08001 or R01AZ08002 was utilized as control for the HPLC analysis. Where noted in the data tables, dissolved oxygen was measured from the stability sample prior to collecting a sample for dilution and HPLC analysis. Formulations were diluted 200-fold in PBS to approximately 0.1 mg/mL for purity analysis of pegnivacogin according to the AX-HPLC.

### C. Ion Pair HPLC Purity Analysis

Ion-pairing HPLC (IP-HPLC) was performed using a Waters Acquity BEH300 C4 column (4.6×100 mm, 3.5 μm). Detection is by UV at 259 nm. The method is equivalent to QC-FPM-0204.

### D. Methionine Assay

HPLC was performed using a Waters OST C18 2.1×50 mm 1.7 μm column at 35° C., 1 μL injection, and a gradient elution over 3 minutes Detection is by UV at 210 nm. The method is capable of detecting reduced and oxidized methionine.

### E. Activity Assay

This assay was performed using a Diagnostica Stago Start 4 manual coagulometer, pooled normal human plasma as a test substrate (e.g. BioMerieux MDA Verify 1 plasma) and BioMerieux MDA AutoaPTT reagent. Pegnivacogin was diluted and added to the test plasma, the aPTT reagent was then added, and the time required for the plasma to clot measured. Over the concentration range of pegnivacogin tested, pegnivacogin yielded a well-defined, saturated dose-response curve.

### F. Dissolved Oxygen Measurement

Dissolved oxygen measurements were obtained according to vendor instructions using a Microx TX3 oxygen meter connected to an external data logger and equipped with a 140 lam silica fiber-optic oxygen microsensor (PreSens Precision Sensing GmbH, Instruction Manual Micron TX3, Josef-Engert-Str. 11, D-93053 Regensburg, Germany, 2006). The oxygen microsensors are capable of measuring in the range of 0 to 22.5 ppm with a limit of detection of 20 ppb. Accuracy is ±1% at 100% air-saturation and ±0.1% at 1% air-saturation. The sensors were calibrated using calibration standards that were freshly prepared.

### Example 3

### Forced Degradation Studies of Pegnivacogin Injection

Forced degradation studies conducted in the presence of 1% H₂O₂, simulating oxidative stress conditions, demonstrated reduction in peak area of the main peak along with significant chromatographic tailing of the RB006. Similar results were obtained after simulated UV stress with an exposure of 3.6 M Lux hours (3×ICH Q1B recommendation). Pegnivacogin was also susceptible to thermal stress as was observed when exposed to 60° C. for 16 hours as a decreased main peak area and broadening on both sides resulted in increased impurity amounts of approximately 5%. The degradation was consistent with a decreased molecular weight due to degraded PEG.

In the presence of peroxide, oxidative damage was presumed to have occurred to the PEG moiety first, followed by damage to the oligonucleotide portion as evidenced by the reduced area (94.46% area vs. 98.16% area for the control) and peak broadening on the front side of the main peak.

In the presence of either UV Stress (3.6 million Lux-hours) or thermal stress (24 hours, 60° C., protected from light), the chromatograms showed similar patterns to the oxidative stressed samples with broadening of the main peak, but at a much slower rate, as expected.

### Example 4

### Formulation Development: Study 1

### A. Buffer Preparation

Phosphate buffers, pH 6.0, 6.5, 7.0 and 7.4 were prepared using sterile water for injection, at a concentration of 66 mM phosphate (equivalent to the clinical formulation), purged with filtered nitrogen gas then filtered separately through a 0.2 micron PVDF Durapore filter. The bulk buffers were stored with an optional nitrogen gas overlay in the headspace until it was time for formulation manufacture.

### B. Factorial Design Formulations

The following variables were explored through the design of eleven (11) different formulations: pegnivacogin concentration, pH, methionine, and EDTA as shown in Table 1. Additionally, use of monothioglycerol, three different stoppers, two vial types and level of dissolved oxygen were evaluated in supplemental formulations as shown in Table 2:

TABLE 1
Pegnivacogin Factorial Design Formulations - Study No. 1

| | | | EDTA | | | |
|---|---|---|---|---|---|---|
| Development | RB006 | Methionine | (% | | | |
| Formulation | (mg/mL) | (% w/w) | w/w) | Stopper¹ | Vial² | pH |
| 040-100 R1 | 25.5 | 0.25 | 0.05 | 1 | 2 | 6.5 |
| 040-100 R2 | 21 | 0.5 | 0 | 1 | 2 | 7.0 |
| 040-100 R3 | 21 | 0 | 0.1 | 1 | 2 | 7.0 |
| 040-100 R4 | 25.5 | 0.25 | 0.05 | 1 | 2 | 6.5 |
| 040-100 R5 | 30 | 0.5 | 0.1 | 1 | 2 | 7.0 |
| 040-100 R6 | 21 | 0.5 | 0.1 | 1 | 2 | 6.0 |
| 040-100 R7 | 30 | 0 | 0 | 1 | 2 | 7.0 |
| 040-100 R8 | 21 | 0 | 0 | 1 | 2 | 6.0 |
| 040-100 R9 | 30 | 0.5 | 0 | 1 | 2 | 6.0 |
| 040-100 R10 | 25.5 | 0.25 | 0.05 | 1 | 1 | 6.5 |
| 040-100 R11 | 30 | 0 | 0.1 | 1 | 2 | 6.0 |

¹West, Stopper 4432/50 Gray Silicone level 3 Westar RS (uncoated)
²Vial 1 = Treated, Type I borosilicate glass, Vial 2 = Untreated, Type I borosilicate glass

TABLE 2
Pegnivacogin Supplemental Formulations - Study No. 1

| Development | RB006 (21 mg/mL) | | | |
|---|---|---|---|---|
| Formulations | Description | Stopper¹ | Vial² | pH |
| 040-100 R12 | Control for CTM | 1 | 1 | 7.4 |
| 040-100 R13 | 1% w/w monothioglycerol | 1 | 1 | 6.0 |
| 040-100 R14 | Teflon ® coated stopper | 2 | 1 | 6.0 |
| 040-100 R15 | FluroTec ® coated stopper | 3 | 1 | 6.0 |
| 040-100 R16 | uncoated stopper | 1 | 1 | 6.0 |
| 040-100 R17 | uncoated stopper, untreated glass | 1 | 2 | 6.5 |

¹Stopper 1 = West, Stopper 4432/50 Gray Silicone level 3 Westar RS (uncoated), Stopper 2 = West, Teflon ® faced stopper 4432/50 Gray Teflon ® 2 silicone level 3, Westar RS, Stopper 3 = Daikyo FluroTec ® closure (S2-F451) D777-1 B2-40 Coat Top surface plug laminated
²Glass 1 = Treated, Type I borosilicate glass, Glass 2 = Untreated, Type I borosilicate glass

### C. Formulation Preparation

Formulation excipients such as sodium chloride and various antioxidants were weighed, added to and mixed with a pre-determined amount of the designated buffer into a previously washed, autoclaved and labeled jar. Nitrogen purging of the bulk solution was carried out where possible throughout manufacture. In-process assays of pegnivacogin content were determined by measuring the UV absorbance at 259 nm and using the following calculation correcting for purity of the Pegnivacogin API lot used.

$$\frac{\text{?}}{Absorbance @ 259 nm} O . D \times \frac{\text{?}}{Purity} \times \frac{\text{?}}{Dilution Factor} \div \frac{\text{?}}{Absorptivity Factor} O . D / mg = \underline{\text{?}} mg / mL$$

$$\text{?} \text{indicates text missing or illegible when filed}$$

If the in-process assay value was acceptable, the pH and osmolality were recorded. If the assay value was too high, the required amount of buffer to add to dilute to target was calculated according to the following equation:

Net wt solution (g)×[(assay−target)/target]=amount of buffer to add to q.s.

After final assay determination, the bulk solution was purged with nitrogen gas for approximately 10 minutes and the headspace for at least an additional 5 minutes. Times may be adjusted for different batch sizes to obtain similar levels of purging with nitrogen gas. The solutions were filtered through a 0.2 μm syringe filter into a sterilized receiving jar for filling. For both studies, the filtered formulation bulk was purged with nitrogen gas during filling. While purging the bulk, each empty vial was pre-purged with filtered nitrogen gas, followed by filling, nitrogen overlay, stopper insertion and crimping of an aluminum flip-off over seal. This process generated the dissolved oxygen target ≦2 ppm. For study 1, a small number of filled nitrogen purged vials from each formulation were unstoppered and purged with 60 mL of air to bring the dissolved oxygen back to ambient conditions. The air-purged vials were then re-stoppered and new crimps applied. This process generated the dissolved oxygen target of ˜10 ppm, which is consistent with the ambient level of dissolved oxygen. For Study 2, as described herein, the dissolved oxygen target of ˜10 ppm was achieved by a direct air purge of the bulk prior to filling and stoppering the vials. The finished vials were then labeled and stored inverted in controlled temperature stability chambers.

### D. Stability Protocol

The filled, stoppered and capped vials of the 17 formulations of Study 1 were stored inverted in controlled temperature stability chambers and tested as outlined in Table 3.

TABLE 3
Stability Protocol for Study 1

| Storage Condition | 0 M | 12 M | 24 M | 36 M |
|---|---|---|---|---|
| Initial | X, Y | | | |
| 5° C. ± 3° C. N2 purged | | X, Y | X, Y | X, Y |
| 5° C. ± 3° C. Air purged | | X, Y | X, Y | X, Y |
| 25° C. ± 2° C./60% ± | | X, Y | X, Y | X, Y |
| 5RH, N2 purged | | | | |
| 40° C. ± 2° C./75% ± | | X, Y | X, Y | X, Y |
| 5RH, N2 purged | | | | |

X= Appearance, pH, DO₂
Y= Assay by UV, Purity by anion exchange chromatography
Purity by ion-pairing chromatography

### Example 5

### Stability Data: Study 1

### A. Stability Study Design

The 11 formulations produced as described in Example 4 were studied on stability under the following four different conditions: (1) 5° C. (<2 ppm dissolved oxygen); (2) 5° C. (ca. 10 ppm dissolved oxygen); (3) 25° C. (<2 ppm dissolved oxygen); and (4) 40° C. (<2 ppm dissolved oxygen). The four-factor, two-level, fractional factorial design with 3 center points (total of 11 runs) is presented in Table 4.

TABLE 4
Four-factor, two-level fractional factorial design

| Factor | Low | High |
|---|---|---|
| [RB006] | 21 mg/mL | 30 mg/mL |
| pH | 6.0 | 7.0 |
| [EDTA] | 0 | 0.1% w/w (1 mg/mL) |
| [Methionine] | 0 | 0.5% w/w (5 mg/mL) |

### B. Overlays of AX-HPLC Stability Data for 040-100-R1-R17 at 12 Months

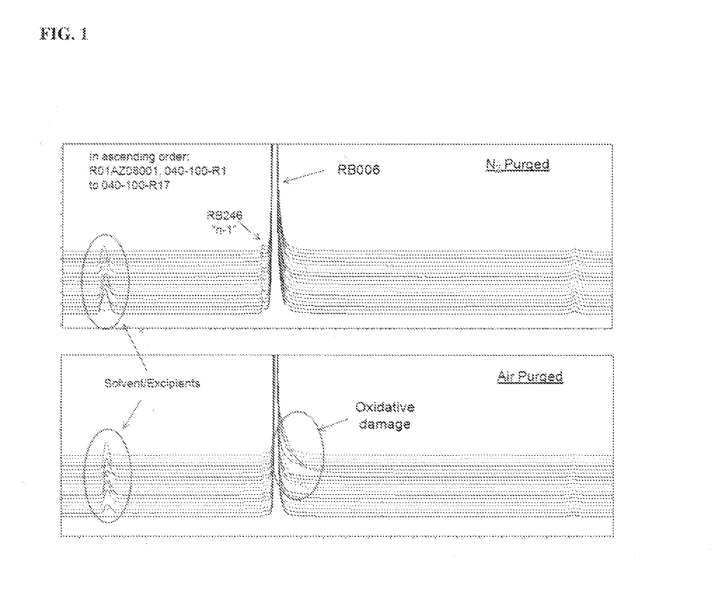

Overlays of the AX-HPLC chromatograms for 12 months storage at 5° C. are presented in FIG. 1. The chromatograms showed virtually no indication of oxidation in the nitrogen purged formulations (top), but clear evidence of oxidative degradation in many of the air purged samples (bottom). In particular, samples from runs 7 & 8 (no antioxidants) and 12-17, showed reduced main peak area and significant tailing.

### C. AX-HPLC Purity Data for 040-100-R1-12 at 12 Months

The corresponding purity data from the AX-HPLC analysis of 040-100-R1-12 after storage for twelve months, grouped in order of increasing methionine concentration, is presented in Table 5.

TABLE 5
AX-HPLC Purity and DO₂ Measurements of Pegnivacogin Formulations 040-100 R1-12; N₂ vs. Air Purged, 5° C.,
25° C. and 40° C for 12 Months

| | | | | | 5° C., | | 5° C., | | 25° C., | | 40° C., | |
|---|---|---|---|---|---|---|---|---|---|---|---|---|
| Pegnivacogin Injection | | | | | N₂ Purged | | Air Purged | | N₂ Purged | | N₂ Purged | |
| Fml# | | | | | AX- | | AX- | DO₂ | AX- | DO₂ | AX- | DO₂ |
| 040- | | | | | HPLC | DO₂ | HPLC | different | HPLC | same | HPLC | same |
| 100- | RB006 | Methionine | EDTA | | (area- | different | (area- | vial | (area- | vial | (area- | vial |
| R-X | (mg/mL) | (% w/w) | (% w/w) | pH | %) | vial (ppm) | %) | (ppm) | %) | (ppm) | %) | (ppm) |
| 8 | 21 | 0 | 0 | 6.0 | 94.89 | 0.488 | 88.80 | 9.88 | 92.95 | 1.37 | 51.21 | 1.29 |
| 7 | 30 | 0 | 0 | 7.0 | 95.11 | 0.454 | 68.39 | 7.95 | 84.64 | 0.852 | 51.37 | 1.18 |
| 11 | 30 | 0 | 0.1 | 6.0 | 95.17 | 0.530 | 93.84 | 9.76 | 93.66 | 1.45 | 43.30 | 0.890 |
| 3 | 21 | 0 | 0.1 | 7.0 | 95.23 | 0.460 | 93.90 | 9.51 | 91.63 | 1.20 | 38.85 | 0.788 |
| 1 | 25.5 | 0.25 | 0.05 | 6.5 | 95.12 | 1.00 | 94.84 | 9.85 | 92.95 | 1.11 | 90.30 | 2.18 |
| 4 | 25.5 | 0.25 | 0.05 | 6.5 | 95.11 | 0.429 | 94.20 | 9.79 | 92.79 | 1.43 | 89.59 | 2.62 |
| 10 | 25.5 | 0.25 | 0.05 | 6.5 | 95.22 | 0.541 | 94.04 | 10.28 | 91.71 | 1.41 | 89.94 | 2.37 |
| 9 | 30 | 0.5 | 0 | 6.0 | 95.42 | 0.442 | 94.22 | 9.84 | 93.91 | 1.43 | 90.59 | 2.45 |
| 2 | 21 | 0.5 | 0 | 7.0 | 94.89 | 1.23 | 94.89 | 9.5 | 92.80 | 1.10 | 86.77 | 2.19 |
| 6 | 21 | 0.5 | 0.1 | 6.0 | 95.67 | 0.414 | 94.33 | 10.55 | 93.30 | 1.16 | 89.74 | 2.31 |
| 5 | 30 | 0.5 | 0.1 | 7.0 | 95.26 | 0.476 | 94.34 | 10.02 | 93.43 | 1.37 | 85.99 | 2.41 |
| 12 | Control | n/a | n/a | 7.4 | 95.16 | 0.518 | 51.8 | 8.22 | 92.43 | 1.30 | 52.94 | 1.46 |
| | for CTM | | | | | | | | | | | |

The AX-HPLC purity results of the nitrogen purged (average DO2˜0.58 ppm) samples stored at 5° C. for 12 months demonstrated insignificant change from the control standard (94% purity) across the range of pegnivacogin concentration (21-30 mg/mL), methionine concentration (0-0.5% w/w), pH (6-7), and EDTA concentration (0-0.1% w/w). From the 12 month purity data at 5° C. it is difficult to distinguish the effect of any given parameter on the stability of pegnivacogin formulations when the formulations were nitrogen purged and stored at 5° C.

It was observed that the purity decreased for the formulations purged with air (DO₂˜8-10 ppm) and stored at 5° C. The AX-HPLC purity decreased by approximately 1% for samples containing methionine and more significantly for samples without methionine as compared to samples purged with N₂. A comparison of air purged samples number 8, 7, and 12 (highlighted in bold in Table 5) representing no methionine or EDTA, demonstrated that pegnivacogin stability in these formulations is pH dependent with increased stability at lower pH: pH 6 (88.80%)>>>pH 7.0 (68.39%)>>pH 7.4 (51.8%). Air purged samples number 11 (93.84%) and 3 (93.90%), containing only 0.1% EDTA demonstrated nearly comparable stability to those samples containing 0.25% or 0.5% methionine (both approximately 95%). This effect of EDTA is evident at the temperature conditions of 5° C. and 25° C.

### D. AX-HPLC Purity Data for 040-100-R12-17 at 12 Months

This group of formulations compared the stability of pegnivacogin under conditions of different container closure components, monothioglycerol, and pH, with results shown in Table 6.

TABLE 6
AX-HPLC Purity and DO₂ Measurements of Pegnivacogin Formulations 040-100 R12-17;
N₂ vs. Air Purged, 5° C., 25° C. and 40° C. for 12 Months

| Pegnivacogin Injection, 21 mg/mL | | | 5° C., N₂ Purged | | 5° C., Air Purged | | 25° C., N₂ Purged | | 40° C., N₂ Purged | |
|---|---|---|---|---|---|---|---|---|---|---|
| Fml# | | | | DO₂ | | DO₂ | | DO₂ | | DO₂ |
| 040- | | | AX-HPLC | same | AX-HPLC | same | AX-HPLC | same | AX-HPLC | same |
| 100- | | Stopper, | (area- | vial | (area- | vial | (area- | vial | (area- | vial |
| R-X | pH | Glass, other | %) | (ppm) | %) | (ppm) | %) | (ppm) | %) | (ppm) |
| 12 | 7.4 | Uncoated, Treated | 95.16 | 0.518 | 51.8 | 8.22 | 92.43 | 1.3 | 52.9 | 1.5 |
| 13 | 6.0 | Uncoated, Treated 1% | 94.53 | 0.06 | 71.94 | 4.6 | 93.99 | 0.02 | 87.2 | n/a |
| | | monothioglycerol | | | | | | | | |
| 14 | 6.0 | Teflon®, Treated | 94.95 | 0.53 | 88.05 | 10.15 | 92.21 | 1.27 | 53.9 | 0.93 |
| 15 | 6.0 | FluroTec®, Treated | 94.96 | 0.75 | 93.69 | 9.87 | 92.01 | 1.15 | 88.7 | 0.09 |
| 16 | 6.0 | Uncoated, Treated | 94.91 | 0.56 | 86.08 | n/a | 94.96 | 1.37 | 50.5 | 1.31 |
| 17 | 6.5 | Uncoated, Untreated | 94.72 | 0.50 | 85.75 | n/a | 89.75 | 1.26 | 53.0 | 1.18 |

The purity data demonstrated that when stored at 5° C. and nitrogen purged, there was no appreciable difference in stability across the formulations at 12 months. Monothioglycerol (1% in formulation 040-100-R13) was shown to be ineffective and somewhat detrimental under the air purged conditions at 5° C., which was limited to only one formulation at pH 6.

The samples with the best measured stability were clearly those with lower pH, low dissolved oxygen and those which included the FluoroTec® coated stopper (regardless if nitrogen or air purged). In the case of the air purged vials (˜10 ppm) 5° C. storage, the FluoroTec coated stopper outperformed the Teflon coated stopper.

### E. Overlays of AX-HPLC Stability Data for 040-100-R1-R17 at 24 Months

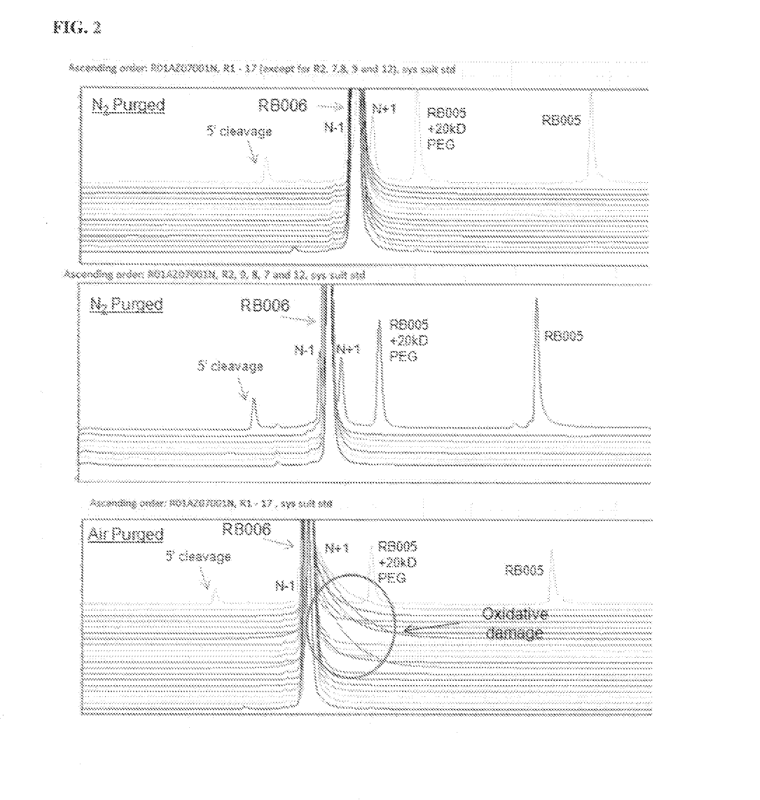

Overlays of the AX-HPLC chromatograms at 24 months, 5° C. storage are presented in FIG. 2. The 24 month chromatograms showed virtually no indication of oxidation in the nitrogen purged formulations (top and middle) demonstrating a 2 year shelf life under these conditions is readily achievable when a sufficient nitrogen purge is employed during manufacturing. As expected, the air purged samples, representing ambient dissolved oxygen levels, (bottom) demonstrated extensive oxidation in a predominance of the samples tested. The middle graph highlights the comparison of R01AZ07001N reference standard to formulations R2 (21 mg/mL, 0.5% Methionine, pH 7), R9 (30 mg/mL, 0.5% methionine, pH 6.0), R8 (21 mg/mL, pH 6.0), R7 (30 mg/mL pH 7.0), and R12 (21 mg/mL, pH 7.4).

### F. AX-HPLC Purity Data for 040-100-R1-12 at 24 Months

AX-HPLC purity data for 040-100-R1-12 after 24 months storage is presented in Table 7.

TABLE 7
AX-HPLC Purity and DO₂ Measurements of Pegnivacogin Formulations 040-100 R1-12; N₂ vs. Air Purged, 5° C.,
25° C. and 40° C. for 24 Months

| Pegnivacogin Injection | | | | | 5° C., N₂ Purged | | 5° C., Air Purged | | 25° C., N₂ Purged | | 40° C., N₂Purged | |
|---|---|---|---|---|---|---|---|---|---|---|---|---|
| Fml# | | | | | | | | DO₂ | | DO₂ | AX- | DO₂ |
| 040- | | | | | AX-HPLC | DO₂ | AX-HPLC | different | AX-HPLC | same | HPLC | same |
| 100- | RB006 | Methionine | EDTA | | (area- | different | (area- | vial | area- | vial | (area- | vial |
| R-X | (mg/mL) | (% w/w) | (% w/w) | pH | %) | vial (ppm) | %) | (ppm) | %) | (ppm) | %) | (ppm) |
| 8 | 21 | 0 | 0 | 6.0 | 93.96 | Not tested | 84.58 | 9.15 | 85.79 | 1.58 | No sample | |
| | | | | | | | | | | | remaining | |
| 7 | 30 | 0 | 0 | 7.0 | 93.50 | Not tested | 47.40 | 7.10 | 75.38 | 1.30 | 35.05 | 1.71 |
| 11 | 30 | 0 | 0.1 | 6.0 | 94.25 | 0.721 | 94.93 | 9.25 | 93.75 | 2.11 | 22.82 | 1.26 |
| 3 | 21 | 0 | 0.1 | 7.0 | 94.62 | 0.593 | 94.53 | 9.63 | 92.50 | 1.52 | 20.69 | 1.14 |

| | | | | | | | | | | | |
|---|---|---|---|---|---|---|---|---|---|---|---|
| 1 | 25.5 | 0.25 | 0.05 | 6.5 | 93.76 | 0.624 | 93.97 | 9.58 | 93.72 | 1.63 | No sample |
| | | | | | | | | | | | remaining |
| 4 | 25.5 | 0.25 | 0.05 | 6.5 | 94.78 | 0.751 | 94.55 | 9.47 | 93.64 | 1.95 | No sample |
| | | | | | | | | | | | remaining |
| 10 | 25.5 | 0.25 | 0.05 | 6.5 | 94.68 | 0.742 | 93.74 | 9.38 | 93.88 | 1.87 | No sample |
| | | | | | | | | | | | remaining |
| 9 | 30 | 0.5 | 0 | 6.0 | 94.57 | 0.745 | 94.57 | 9.36 | 94.17 | 1.85 | No sample |
| | | | | | | | | | | | remaining |
| 2 | 21 | 0.5 | 0 | 7.0 | 93.09 | 0.443 | 94.48 | 9.67 | 92.78 | 1.89 | No sample |
| | | | | | | | | | | | remaining |
| 6 | 21 | 0.5 | 0.1 | 6.0 | 94.80 | 0.694 | 95.16 | 9.49 | 93.61 | 1.70 | No sample |
| | | | | | | | | | | | remaining |
| 5 | 30 | 0.5 | 0.1 | 7.0 | 94.50 | 0.715 | 94.33 | 9.26 | 93.47 | 1.84 | No sample |
| | | | | | | | | | | | remaining |
| 12 | Control | n/a | n/a | 7.4 | 93.81 | Not tested | 40.79 | 6.88 | 74.35 | 1.32 | No sample |
| | for CTM | | | | | | | | | | remaining |

Consistent with the data generated after 12 months storage, the 24 month samples stored at 5° C. and nitrogen purged demonstrated very little degradation, even for formulations 040-100-R7 and R8 that contained no antioxidants. At 5° C./air purged or 25° C./nitrogen purged, formulations containing methionine and/or EDTA remained within current AX-HPLC purity specification of 92%. Formulation 040-100-R7 and R8 without antioxidant were the only formulations that fell below the purity specification under those conditions. Comparison of formulation 040-100-7 (pH 7) and formulation R8 (pH 6) at 5° C./air purged at the accelerated conditions showed that the lower pH 6 formulation consistently demonstrated better stability.

### G. AX-HPLC Purity Data for 040-100-R12-17 at 24 Months

AX-HPLC purity data for 040-100-R12-17 after 24 months storage is presented in Table 8. As indicated above, this group of formulations compared the stability of pegnivacogin under conditions of different container closure components, monothioglycerol, and pH.

TABLE 8
AX-HPLC Purity and DO₂ Measurements of Pegnivacogin Formulations
040-100 R12-17; N₂ vs. Air Purged, 5° C., 25° C. and 40° C. for 24 Months

| Pegnivacogin Injection, | | | 5° C., | | 5° C., | | 25° C., | | 40° C., | |
|---|---|---|---|---|---|---|---|---|---|---|
| 21 mg/mL | | | N₂ Purged | | Air Purged | | N₂ Purged | | N₂ Purged | |
| Fml# | | | AX- | DO₂ | AX- | DO₂ | AX- | DO₂ | AX- | DO₂ |
| 040- | | | HPLC | same | HPLC | same | HPLC | same | HPLC | same |
| 100- | | Stopper, | (area- | vial | (area- | vial | (area- | vial | (area- | vial |
| R-X | pH | Glass, other | %) | (ppm) | %) | (ppm) | %) | (ppm) | %) | (ppm) |
| 12 | 7.4 | Uncoated, Treated | 93.81 | NT | 40.79 | 6.88 | 74.35 | 1.32 | No sample | |
| | | | | | | | | | remaining | |
| 13 | 6.0 | Uncoated, Treated | 93.64 | 0.038 | 64.43 | 3.56 | 94.04 | 0.052 | 83.89 | 0.042 |
| | | 1% monothioglycerol | | | | | | | | |
| 14 | 6.0 | Teflon®, Treated | 94.48 | 0.655 | 77.99 | 8.75 | 93.92 | 1.50 | No sample | |
| | | | | | | | | | remaining | |
| 15 | 6.0 | FluroTec®, Treated | 94.22 | 0.683 | 93.30 | 8.80 | 93.62 | 0.177 | 89.08 | 0.075 |

| | | | | | | | | | |
|---|---|---|---|---|---|---|---|---|---|
| 16 | 6.0 | Uncoated, Treated | 94.28 | 0.845 | 76.15 | 8.74 | 69.78 | 1.26 | No sample |
| | | | | | | | | | remaining |
| 17 | 6.5 | Uncoated, Untreated | 94.50 | 0.704 | 56.26 | 7.87 | 76.77 | 1.44 | No sample |
| | | | | | | | | | remaining |

Consistent with the 12 month data, the purity data demonstrated that when stored at 5° C. and nitrogen purged, there was no appreciable difference in stability across the formulations at 24 months.

### H. Overlays of AX-HPLC Stability Data for 040-100-R12 at 12 Months

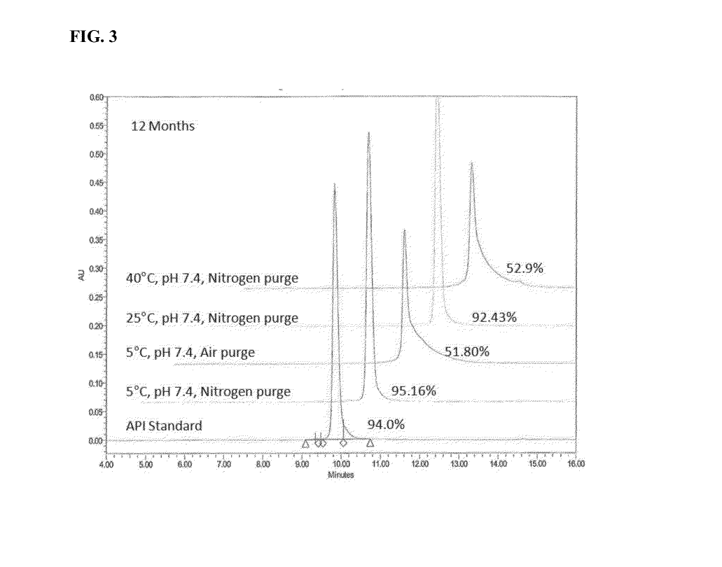

Overlays of representative AX-HPLC chromatograms formulation 040-100-R12 (Clinical control at pH 7.4) after storage for 12 months at various conditions are presented in FIG. 3. The benefit of reducing the dissolved oxygen to less than 2 ppm in the bulk solution prior to filtration and vial filling is readily observed by comparing the results of the AX-HPLC analysis of samples stored for 12 months at 5° C. processed with and without nitrogen purge. Chromatograms of samples processed without nitrogen purge but stored at 5° C. demonstrated reduced purity (51.8%) and broadened area of the main peak, along with a greater degree of late eluting or co-eluting peaks. This chromatographic behavior is consistent with PEG oxidative degradation observed in forced degradation studies using 1% H₂O₂ discussed earlier in this report. While nitrogen purging of formulations stored at 25° C. provided almost comparable stability to 5° C. storage, nitrogen purging was not effective to the same extent for pegnivacogin formulations stored at 40° C.

### I. IP-HPLC Stability Data for 040-100-R1-17 at 12 and 24 Months

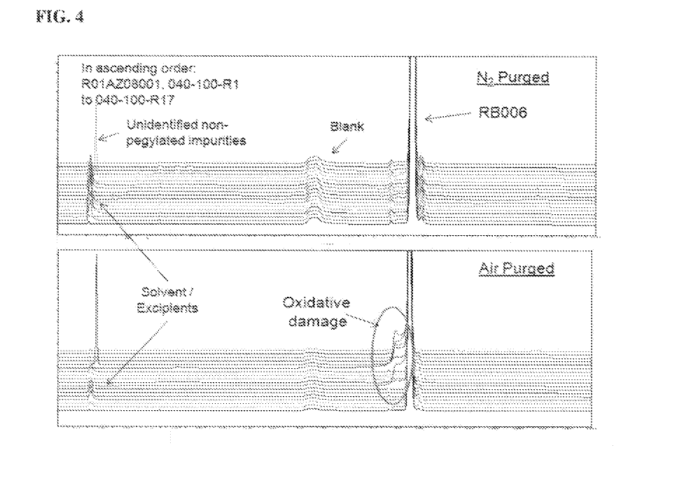

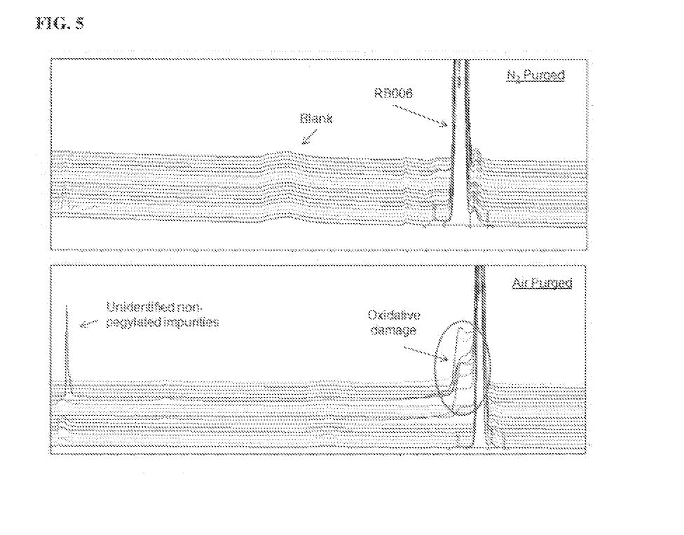

Overlay IP-HPLC chromatograms of the same formulations for the 5° C. storage samples for 12 and 24 months, are presented in FIGS. 4 and 5, respectively. The IP-HPLC chromatograms also show similar trends as the AX-HPLC analysis with respect to enhanced stability in all the nitrogen purged samples while oxidative damage is evident in many of the air purged samples.

At 36 months, all 5° C. N2 purged formulations were within purity and impurity specifications. 5° C. Air purged formulations R1 through R6, R7 through R10 and R15 met specifications while degradation was evident in the remaining formulations. At the 25° C. N2 purged condition, only formulations R1, R2, R4 through R6, R9 through R11, R13 and R15 met specifications. Formulations of interest, R2 and R9 met current IP-HPLC specifications when stored for 36 months at 5° C. N2 purged, 5° C. air purged, and 25° C. N2 purged conditions.

### J. AX-HPLC Stability Data at 36 Months

AX-HPLC purity measurements and dissolved oxygen measurements for the 36 month time point are presented in Table 9 and Table 10. Purity values for formulations having one or more impurity measurements out of specification are designated with a footnote in the data tables. After 36 months at the storage condition 5° C. N₂ purged, the following eight formulations remain within specifications for AX-HPLC purity and impurities: R1-R5, R7, R9, and R11. Of these formulations, all include methionine and/or EDTA with the exception of R7 (30 mg/mL, pH 7.0). Of the formulations still within specifications, R1-R4 are the most stable. Formulations most pertinent to discussion are those with concentration and pH values that bracket the Phase 3 clinical formulation and contain methionine without EDTA. Those formulations are R2 (21 mg/mL, pH 7.0, 0.5% methionine) and R9 (30 mg/mL, pH 6.0, 0.5% methionine). R2 has good stability and meets current AX-HPLC specifications when stored for 36 months at 5° C. N₂ purged, 5° C. air purged, and 25° C. N₂ purged. R9 meets AX-HPLC specifications for 5° C. N₂ purged (although near the limit sum RRT≧1.01≦6.0 and no individual >5.5 area-%) and for 25° C. N₂ purged condition, but not surprisingly failed this same specification at the 5° C. air purged condition.

TABLE 9
AX-HPLC Purity and DO2 Measurements of Pegnivacogin Formulations 040-100 R1-12;
N2 vs. Air Purged, 5° C. and 25° C. for 36 Months

| Pegnivacogin Injection | | | | | 5° C. N₂ Purged | | 5° C., Air Purged | | 25° C., N₂ Purged | |
|---|---|---|---|---|---|---|---|---|---|---|
| Fml# | | | | | AX- | DO₂ same | AX- | DO₂ same | AX- | DO₂ same |
| 040- | RB006 | Methionine | EDTA | | HPLC^{a)} | vial | HPLC^{a)} | vial | HPLC^{a)} | vial |
| 100-R-X | (mg/mL) | (% w/w) | (% w/w) | pH | (area-%) | (ppm) | (area-%) | (ppm) | (area-%) | (ppm) |
| 8 | 21 | 0 | 0 | 6.0 | 92.69^{b)} | 0.997 | 67.32^{b)c)} | 8.48 | 64.03^{b)c)} | 2.28 |
| 7 | 30 | 0 | 0 | 7.0 | 94.32 | 1.08 | 38.02^{b)c)} | 7.28 | 63.30^{b)c)} | 1.78 |
| 11 | 30 | 0 | 0.1 | 6.0 | 94.24 | 1.09 | 94.83 | 9.82 | 94.26 | 2.97 |
| 3 | 21 | 0 | 0.1 | 7.0 | 94.93 | 0.861 | 94.20 | 9.73 | 92.82 | 2.11 |
| 1 | 25.5 | 0.25 | 0.05 | 6.5 | 95.02 | 0.769 | 94.11 | 10.44 | 93.01^{b)} | 2.52 |
| 4 | 25.5 | 0.25 | 0.05 | 6.5 | 94.71 | 0.978 | 94.24 | 9.58 | 93.96 | 2.85 |
| 10 | 25.5 | 0.25 | 0.05 | 6.5 | 93.93^{b)} | 0.954 | 94.58 | 10.07 | 93.79 | 2.94 |
| 9 | 30 | 0.5 | 0 | 6.0 | 94.05 | 1.05 | 93.70 | 9.62 | 93.88 | 3.33 |
| 2 | 21 | 0.5 | 0 | 7.0 | 94.92 | 0.414 | 94.28 | 10.04 | 94.55 | 2.77 |
| 6 | 21 | 0.5 | 0.1 | 6.0 | 94.04^{b)} | 0.916 | 93.62^{b)} | 9.97 | 94.83 | 2.54 |
| 5 | 30 | 0.5 | 0.1 | 7.0 | 94.43 | 1.110 | 94.23 | 9.92 | 92.95 | 3.07 |
| 12 | Control | n/a | n/a | 7.4 | 92.99^{b)} | 1.03 | 36.10^{b)c)} | 7.56 | 65.35^{b)c)} | 1.91 |
| | for CTM | | | | | | | | | |

^{a)}Control API purity was 94.60 area-%
^{b)}designates sum RRT ≧1.01 ≦6.0 and no individual >5.5 is out of specification.
^{c)}designates overall purity and/or one or more individual impurities is out of specification.

TABLE 10
AX-HPLC Purity and DO2 Measurements of Pegnivacogin Formulations 040-100 R12-17;
N2 vs. Air Purged, 5° C. and 25° C. for 36 Months

| Pegnivacogin Injection, 21 mg/mL | | | 5° C., N₂ Purged | | 5° C., Air Purged | | 25° C., N₂ Purged | |
|---|---|---|---|---|---|---|---|---|
| Fml# | | | AX- | DO₂ same | AX- | DO₂ same | AX- | DO₂ same |
| 040-100- | | Stopper, | HPLC^{a,b} | vial | HPLC^{a,c} | vial | HPLC^{a} | vial |
| R-X | pH | Glass, other | (area-%) | (ppm) | (area-%) | (ppm) | (area-%) | (ppm) |
| 12 | 7.4 | Uncoated, Treated | 92.99 | 1.03 | 36.10 | 7.56 | 65.35^{c} | 1.91 |
| 13 | 6.0 | Uncoated, Treated | 92.21 | 0.464 | 67.19 | 3.63 | 89.10^{c} | 0.352 |
| | | 1% monothioglycerol | | | | | | |
| 14 | 6.0 | Teflon®, Treated | 92.72 | 0.935 | 57.65 | 8.66 | 65.56^{c} | 1.58 |
| 15 | 6.0 | FluroTec®, Treated | 92.75 | 1.01 | 86.88 | 8.62 | 92.85^{b} | 0.053 |
| 16 | 6.0 | Uncoated, Treated | 91.85^{c} | 1.15 | 59.64 | 8.60 | 68.83^{c} | 1.99 |
| 17 | 6.5 | Uncoated, Untreated | 91.85^{c} | 0.953 | 37.97 | 7.56 | 67.10^{c} | 1.81 |

^{a}Control API purity was 94.60 area-%
^{b}designates sum RRT ≧1.01 ≦6.0 and no individual >5.5 is out of specification for all values
^{c}designates overall purity and/or one or more individual impurities is out of specification

Collectively, these data suggest that the formulation conditions selected for phase 3 should produce shelf-life of at least 36 months at 5° C. or 25° C., if adequately N₂ purged. The presence of methionine or EDTA is critical to achieving adequate shelf-life at 5° C. or 25° C., regardless of the level of dissolved oxygen, however the lower the dissolved oxygen, the greater the stability enhancement and the longer the expected shelf-life.

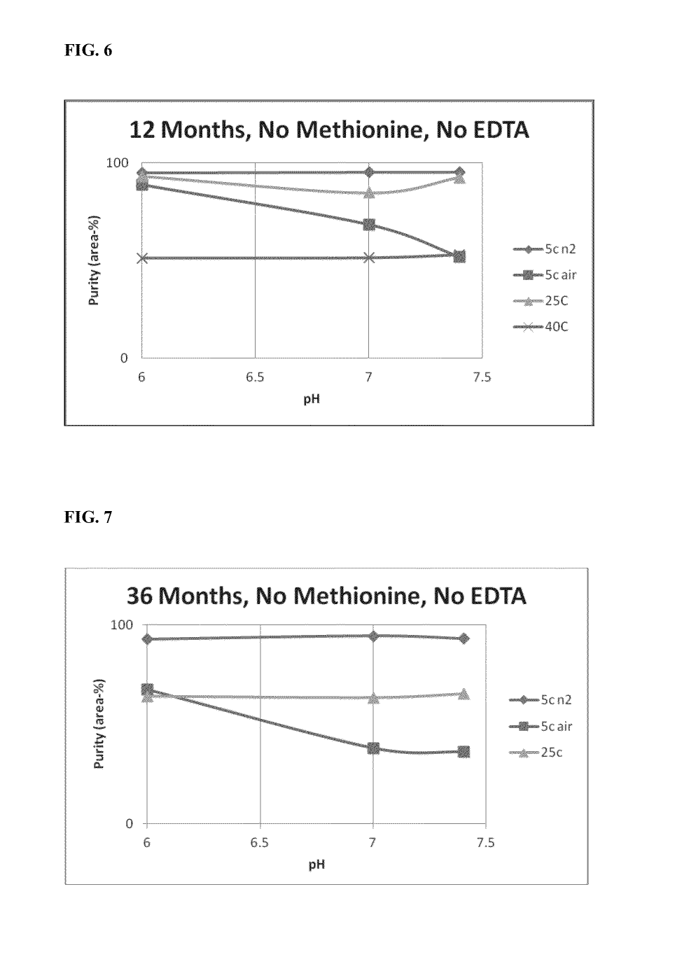

FIGS. 6 and 7 demonstrate the destabilizing effect of temperature and air (dissolved oxygen ˜10 ppm) in the absence of an antioxidant on RB006 formulation purity over 12 and 36 months, respectively. This destabilization was pH dependent, whereby the stability increased as pH decreased. This pH effect was most observable under the 5° C./air purged condition (at 12 and 36 months), and to a lesser extent at 25° C./nitrogen purged (36 months). This pH effect was also evident at the 12 month time point, as the increased purity of the pH 7.4 formulation was believed to be an anomalous result.

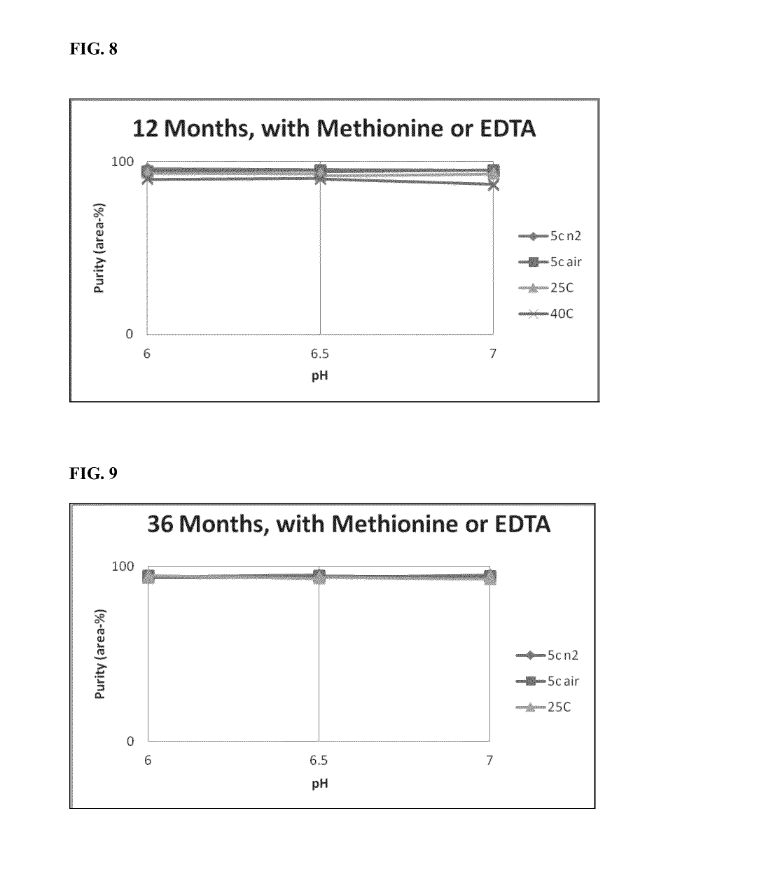

In the presence of either EDTA or methionine at 12 months (FIG. 8) and 36 months (FIG. 9) there was little difference in stability and the stability was independent of pH and dissolved oxygen for the 5° C. storage condition.

Methionine content was measured in those formulations containing methionine (R1, R4, R5, R6, and R10). Methionine content after 36 months at all conditions was consistent with initial values within the analytical variability of the method. The relatively unchanged methionine content suggests sufficient overage of methionine is present in the formulations even when air purged which is equivalent to ambient levels of dissolved oxygen. Methionine is present at 0.25% w/w and 0.5% w/w in the Study 1 formulations while the formulation at ambient dissolved O₂ was established at 0.1% w/w.

### Example 6

### Oligonucleotide Binding Activity

Formulations of pegnivacogin injection, with and without methionine, across the concentration, pH and storage conditions evaluated in Example 4 as well as a sample from the clinical batch 006AT1109CPS as a control, were evaluated for anticoagulant activity.

The anticoagulant activity of pegnivacogin is dependent upon the primary sequence, secondary and tertiary structure of the oligonucleotide and was measured using a standard aPTT plasma-based assay used for both the drug substance and drug product release.

The calculated results from the generated aPTT dose response curves for reference standard (Lot# R01AZ04004N) as compared to the respective formulation samples are presented in Table 11.

TABLE 11
Anticoagulant Activity Results for Pegnivacogin Formulations

| | | | | | |
|---|---|---|---|---|---|
| | | | | | Anticoagulant |
| | Methionine | Storage | N₂ | API ID | Activity |
| Formulation ID | (% w/w) | Condition | Purged | R01AZ-X | Spec = 0.6-1.4 |

21 mg/mL, pH 7.0

| | | | | | |
|---|---|---|---|---|---|
| 040-100R2 | 0.5 | 36 months, 5° C. | Yes | 8001 | 1.043 |
| | 0.5 | 36 months, 5° C. | No | 8001 | 1.000 |
| | 0.5 | 36 months, 25° C. | Yes | 8001 | 1.067 |
| 086-043A | 0.5 | Initial, 5° C. | Yes | 8001 | 1.000 |

30 mg/mL, pH 6.0

| | | | | | |
|---|---|---|---|---|---|
| 040-100R9 | 0.5 | 36 months, 5° C. | Yes | 8001 | 0.964 |
| | 0.5 | 36 months, 5° C. | No | 8001 | 0.970 |
| | 0.5 | 36 months, 25° C. | Yes | 8001 | 0.970 |
| 086-043A | 0.5 | Initial, 5° C. | Yes | 8001 | 1.000 |

Stability samples of formulations R2 and R9 after 36 months storage at the three stability conditions mentioned above, along with freshly prepared controls of each formulation were analyzed using the bioactivity assay for pegnivacogin. The results of the bioactivity assay (Table 11) compared favorably with controls for both formulations at all conditions, demonstrating that all formulations retained their full anticoagulant activity throughout 36 months of storage at the three storage conditions. Retention of full anticoagulant activity confirms that the integrity of the oligonucleotide portion of pegnivacogin was maintained during these storage conditions.

### Example 7

### Formulation Development—Study 2

### A. Experimental Design

Two groups of formulations were prepared. Formulations in the first group were intended to evaluate the effect of the container closure system at different pH and dissolved oxygen levels. Formulations in the second group were designed to evaluate the effect of methionine at different concentrations using the same container closure system.

Group 1 included two formulations: Formulation A, buffered at pH 7.4 and Formulation B, pH 6.5. The two formulations were prepared in bulk, filtered, and filled into the container closure system, and processed with the designated target DO₂ according to Table 12 and 13.

TABLE 12
Pegnivacogin Injection, 21 mg/mL, Study 2, Group 1A

| Formulations | | Meth- | | | | Target |
|---|---|---|---|---|---|---|
| (073-125 R1-12) | | ionine | Stopper | | Target | DO₂ |
| Formulation | Run # | (% w/w) | Type¹ | Glass² | pH | (ppm) |
| A | 1 | 0 | 1 | 1 | 7.4 | ≦1 |
| | 2 | 0 | 2 | 1 | 7.4 | ≦1 |
| | 3 | 0 | 3 | 1 | 7.4 | ≦1 |
| | 4 | 0 | 1 | 2 | 7.4 | ≦1 |
| | 5 | 0 | 2 | 2 | 7.4 | ≦1 |
| | 6 | 0 | 3 | 2 | 7.4 | ≦1 |
| | 7 | 0 | 1 | 1 | 7.4 | 10.0 |
| | 8 | 0 | 2 | 1 | 7.4 | 10.0 |
| | 9 | 0 | 3 | 1 | 7.4 | 10.0 |
| | 10 | 0 | 1 | 2 | 7.4 | 10.0 |
| | 11 | 0 | 2 | 2 | 7.4 | 10.0 |
| | 12 | 0 | 3 | 2 | 7.4 | 10.0 |

¹Stopper 1 = West, Stopper 4432/50 Gray Silicone level 3 Westar RS (uncoated), Stopper 2 = West Stopper w/ B2 crosslinked silicon on FluroTec ® coated West 4432/50 Stopper 3 = West Stopper (4432/50) with B2 crosslinked silicon
²Glass 1 = Treated, Type I borosilicate glass, Glass 2 = Untreated, Type I borosilicate glass

TABLE 13
Pegnivacogin Injection, 21 mg/mL, Study 2, Group 1B

| Formulations | | Meth- | | | | Target |
|---|---|---|---|---|---|---|
| (073-125 R13-24) | | ionine | Stopper | | Target | DO₂ |
| Formulation | Run # | (% w/w) | Type¹ | Glass² | pH | (ppm) |
| B | 13 | 0 | 1 | 1 | 6.5 | ≦1 |
| | 14 | 0 | 2 | 1 | 6.5 | ≦1 |
| | 15 | 0 | 3 | 1 | 6.5 | ≦1 |
| | 16 | 0 | 1 | 2 | 6.5 | ≦1 |
| | 17 | 0 | 2 | 2 | 6.5 | ≦1 |
| | 18 | 0 | 3 | 2 | 6.5 | ≦1 |
| | 19 | 0 | 1 | 1 | 6.5 | 10.0 |
| | 20 | 0 | 2 | 1 | 6.5 | 10.0 |
| | 21 | 0 | 3 | 1 | 6.5 | 10.0 |
| | 22 | 0 | 1 | 2 | 6.5 | 10.0 |
| | 23 | 0 | 2 | 2 | 6.5 | 10.0 |
| | 24 | 0 | 3 | 2 | 6.5 | 10.0 |

¹Stopper 1 = West, Stopper 4432/50 Gray Silicone level 3 Westar RS (uncoated), Stopper 2 = West Stopper w/ B2 crosslinked silicon on FluroTec ® coated West 4432/50 Stopper 3 = West Stopper (4432/50) with B2 crosslinked silicon
²Glass 1 = Treated, Type I borosilicate glass, Glass 2 = Untreated, Type I borosilicate glass

Formulation samples from Study 2, Group 1A and 1B were stored inverted.

Formulations in the second group were prepared as outlined in outlined in Table 14.

TABLE 14
Pegnivacogin Injection, 21 mg/mL, Study 2, Group 2

| Formulations | | | | | | |
|---|---|---|---|---|---|---|
| (073-125 R25-31) | | | | | | Target |
| | Run | Methionine | Stopper | | Target | DO₂ |
| Formulation | # | (% w/w) | Type¹ | Glass² | pH | (ppm) |
| A | 25 | 0 | 1 | 2 | 7.4 | ≦1 |
| C | 26 | 0.1 | 1 | 2 | 6.5 | ≦1 |
| D | 27 | 0.25 | 1 | 2 | 6.5 | ≦1 |
| C | 28 | 0.1 | 1 | 2 | 6.5 | 10 |
| D | 29 | 0.25 | 1 | 2 | 6.5 | 10 |
| E | 30 | 0.1 | 1 | 2 | 7.4 | ≦1 |
| F | 31 | 0.1 | 1 | 2 | 7.0 | ≦1 |

¹1 = West, Stopper 4432/50 Gray Silicone level 3 Westar RS (uncoated)
²2 = Untreated, Type I borosilicate glass

### B. Stability Protocols

Group 2 were stored inverted. A 50° C. condition was added to this group to facilitate differentiation of the various formulations in a shorter duration study.

The stability protocols for the two formulations of Group 1 and the formulations of Group 2, respectively, are shown in Tables 15 and 16:

TABLE 15
Stability Protocol for Study 2, Group 1A and 1B

| Storage Condition | 0 M | 10.5 M | 12 M | 24 M | 36 M |
|---|---|---|---|---|---|
| Initial | X | | | | |
| 5° C. ± 3° C. | | | X | X | X |
| 25° C. ± 2° C./60% ± | | | X | X | |
| 5RH | | | | | |
| 40° C. ± 2° C./75% ± | | X | X | | |
| 5RH | | | | | |

TABLE 16
Stability Protocol for Study 2, Group 2

| Storage Condition | 0 M | 3 M | 6 M | 12 M | 24 M | 36 M |
|---|---|---|---|---|---|---|
| Initial | X | | | | | |
| 5° C. ± 3° C. | | | | | X | X |
| 40° C. ± 2° C./75% ± | | X | X | X | | |
| 5RH | | | | | | |
| 50° C. ± 2° C. | | X | X | X | | |

Stability data from the three and six month time points were generated.

The analysis of stability samples from Study 2 formulations designed to compare the effects of different container closure configurations and stored under the conditions of pH 6.5 vs. 7.4, N₂ purged vs. air purged, stored for 12, and 24 months at 5° C. and 25° C., or 10.5 months (replaced 9 month pull) and 12 months at 40° C. is presented. More interestingly, the auxiliary formulations (R25-R31) containing 0.1% vs. 0.25% methionine, pH 6.5 vs. 7.4 and N₂ purged vs. air purged and stored in untreated glass, stored for 10.5 and 12 months at 40° C., and 24 months at 5° C., is reviewed. Stability samples were analyzed for AX-HPLC purity and impurities, dissolved oxygen, and methionine content where relevant.

### Example 8

### Stability Data

### A. AX-HPLC Stability Data for 040-125 R25-31 at 3 Months

Pegnivacogin purity analysis by AX-HPLC, % methionine and dissolved oxygen content after storage at 40° C. and 50° C. for 3 months are presented in Table 17:

TABLE 17
AX-HPLC Purity, % Methionine, and DO₂ Measurements of Pegnivacogin
Formulations 040-125 R25-31; 40° C. and 50° C. for 3 Months.

| | | | | | | | 40° C./75% RH | | | 50° C. | | |
|---|---|---|---|---|---|---|---|---|---|---|---|---|
| Formulations | | | | | | Target | AX- | | | AX- | | |
| (073-125 R25-31) | | Methionine | Stopper | | Target | DO₂ | HPLC | Methionine | DO₂ | HPLC | Methionine | DO₂ |
| Fml ID | Run # | (% w/w) | Type¹ | Glass² | pH | (ppm) | (area-%) | (%) | (ppm) | (area-%) | (%) | (ppm) |
| A | 25 | 0 | 1 | 2 | 7.4 | ≦1 | 93.37 | n/a | 0.6177 | 43.40 | n/a | 1.76 |
| C | 26 | 0.1 | 1 | 2 | 6.5 | ≦1 | 94.76 | 105 | 0.7390 | 77.10 | 107.00 | 1.91 |
| D | 27 | 0.25 | 1 | 2 | 6.5 | ≦1 | 94.09 | 103.6 | 0.7068 | 73.67 | 104.40 | 1.74 |
| C | 28 | 0.1 | 1 | 2 | 6.5 | 10 | 93.53 | 104 | 10.69 | 49.91 | 65.00 | 8.14 |
| D | 29 | 0.25 | 1 | 2 | 6.5 | 10 | 93.58 | 104.8 | 10.80 | 54.50 | 83.60 | 7.79 |
| E | 30 | 0.1 | 1 | 2 | 7.4 | ≦1 | 94.36 | 97.0 | 0.7464 | 69.45 | 91.00 | 1.82 |
| F | 31 | 0.1 | 1 | 2 | 7.0 | ≦1 | 94.54 | 102 | 0.7290 | 81.33 | 104.00 | 1.72 |

¹= West, Stopper 4432/50 Gray Silicone level 3 Westar RS (uncoated)
²= Untreated, Type I borosilicate glass

After 3 months under these conditions, the only discriminating changes were observable at 50° C. Specifically, the effect of lower pH on improved pegnivacogin stability in absence of antioxidants or nitrogen purge was noted from the results in Example 5. Here, this trend is generally noted in the AX-HPLC analysis of pegnivacogin formulations, whereby formulations at 21 mg/mL containing 0.1% methionine at different pH values, 6.5, 7.0 and 7.4, at 50° C., have purity values of 77.1%, 81.33%, and 69.5%.

Under these stressed study conditions, the RRT of the observed degradation peaks were consistent with peaks for the 3′ cleavage, 5′ cleavage, and the RB005+20 kD PEG product. Notably, these degradants are different from those observed as tails in the AX-HPLC analysis, and these degradants are not a result of oxidation of PEG. Pegnivacogin formulations appeared most stable at pH 7 with the following rank order of stability in terms of main peak purity: pH 7 (81.33%)>pH 6.5 (77.1%)>pH 7.4 (69.5%). The pH effect is relatively flat for the degradation band of peaks Sum RRT≦0.99, however the degradation Sum RRT≧1.01, representing degradants related to oxidative degradation of PEG, rises sharply at pH 7.4.

Also noteworthy, the methionine content in the formulations remained relatively unchanged, in samples stored for 3 months at 40° C./75% RH regardless if nitrogen purged, and at 50° C. if nitrogen purged. For air purged samples stored at 50° C. for 3 months, there was approximately 16% and 35% decreased in the methionine content in samples containing 0.25% and 0.1% methionine.

### B. AX-HPLC Stability Data for 040-125 R25-31 at 6 Months

At the 6 months time point, only the 40° C./75% RH (Table 18) samples were evaluated.

TABLE 18
AX-HPLC Purity, % Methionine, and DO₂ Measurements of Pegnivacogin
Formulations 040-125 R25-31; 40° C. for 6 Months.

| Formulations | | | | | | | | | |
|---|---|---|---|---|---|---|---|---|---|
| (073-125 R25-31) | | | | | | Target | 40° C./75% RH | | |
| Fml | | Methionine | Stopper | | Target | DO₂ | AX-HPLC | Methionine | DO₂ |
| ID | Run # | (% w/w) | Type¹ | Glass² | pH | (ppm) | (area-%) | (% w/w) | (ppm) |
| A | 25 | 0 | 1 | 2 | 7.4 | ≦1 | 78.70 | N/A | 0.618 |
| C | 26 | 0.1 | 1 | 2 | 6.5 | ≦1 | 92.20 | 110 | 0.739 |
| D | 27 | 0.25 | 1 | 2 | 6.5 | ≦1 | 92.30 | 104 | 0.707 |
| C | 28 | 0.1 | 1 | 2 | 6.5 | 10 | 88.87 | 100 | 10.69 |
| D | 29 | 0.25 | 1 | 2 | 6.5 | 10 | 86.33 | 104 | 10.80 |
| E | 30 | 0.1 | 1 | 2 | 7.4 | ≦1 | 92.58 | 100 | 0.746 |
| F | 31 | 0.1 | 1 | 2 | 7.0 | ≦1 | 93.51 | 110 | 0.729 |

¹= West, Stopper 4432/50 Gray Silicone level 3 Westar RS (uncoated)
²= Untreated, Type I borosilicate glass

Formulations containing methionine and a reduced pH are protective as compared to the existing formulation at the accelerated study condition of 40° C. and at 6 months regardless if the samples had low or high dissolved oxygen levels. Clearly, a reduction of oxygen improved the stability of the formulations containing methionine at pH 6.5 under stressed conditions. It is important to note that without a reduction in oxygen under stressed conditions, there is evidence of methionine oxidation and consumption under the highly stressed condition of 50° C.

A reduced level of 0.1% methionine, i.e., 0.1%, was shown to be effective. As shown in Table 17, at an elevated temperature of 50° C., with the presence of 0.1% methionine, pegnivacogin stability at 3 months is improved from 43.4% to 69.45%, demonstrating the protective effect of methionine.

### C. AX-HPLC Stability Data for 040-125 R25-31 at 12 Months

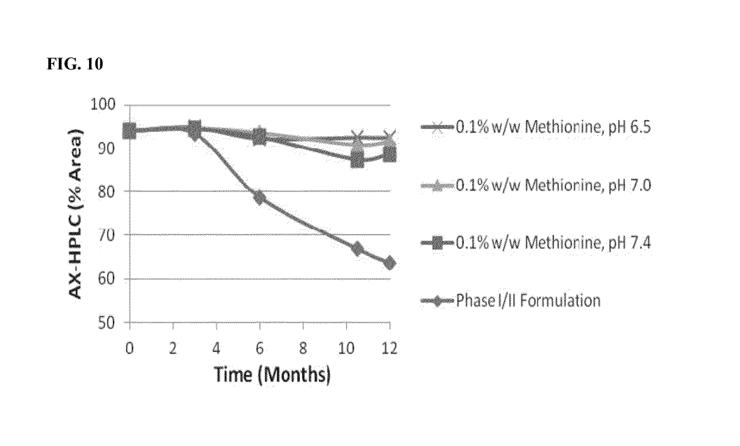

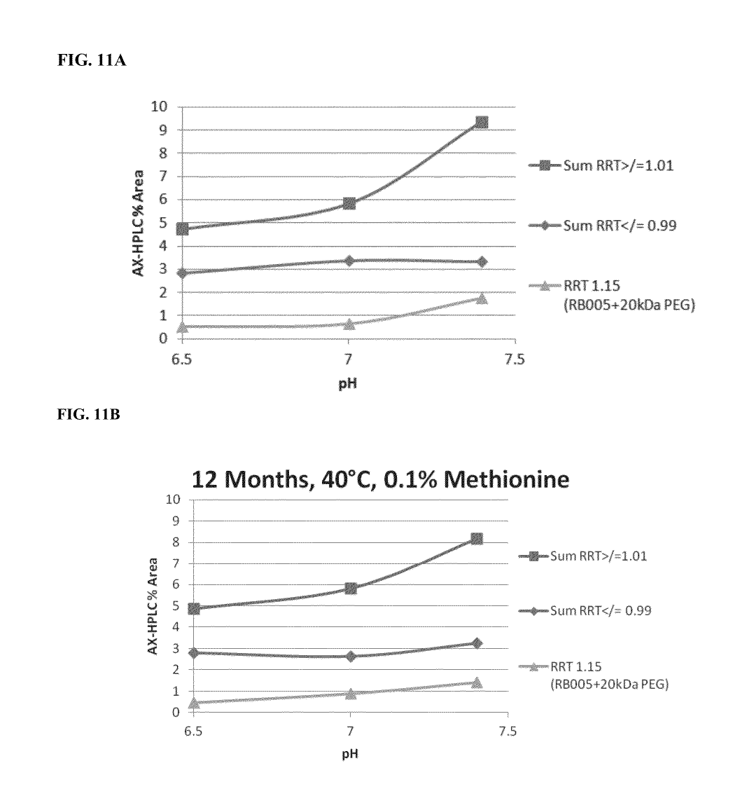

The AX-HPLC purity values, % methionine, and dissolved oxygen measurements for the accelerated samples containing methionine (R26-R31) and the control formulation (R25) at 10½ and 12 months at 40° C. storage are presented in Table 19 and Table 20. The best stability, approximately 92% purity, was observed for R26 and R27 samples, formulated at pH 6.5 with methionine at 0.1 and 0.25%, respectively. A slightly lower purity of approximately 91% was obtained for formulation R31, pH 7.0 and 0.1% w/w methionine. The overall effect of the addition of 0.1% w/w methionine and pH on the stability of pegnivacogin in the drug product at 40° C. is depicted in FIG. 10. The data presented shows that in the addition of 0.1% w/w methionine across the pH range of 6.5 to 7.4 significantly improved the stability of pegnivacogin in the drug product when stored at accelerated conditions of 40° C./75% RH for up to 12 months. A comparison of the effect of pH on the formation of impurities at 10½ and 12 months, 40° C. for formulations containing 0.1% w/w methionine is shown in FIG. 11A and FIG. 11B, At both time points, the effect of pH was the comparable across the pH range from 6.5 to 7.0 for both sum RRT≦0.99 and sum RRT≧1.01, however the degradation sum RRT≧1.01 rises sharply at pH 7.4, very similar to the relationship observed at 3 months, 50° C. A slight pH effect is observed for RRT 1.15 (RB005+20 kDa PEG), consistent with base stressed samples. The methionine content remained relatively unchanged for samples stored for up to 12 months at 40° C./75% which suggested sufficient overage of methionine was present in the formulations even at the 0.1% w/w methionine concentration.

TABLE 19
AX-HPLC Purity, % Methionine, and DO2 Measurements of Pegnivacogin
Formulations 040-125 R25-31; 40° C. for ~10.5 Months.

| Formulations | | | | | | Target | 40° C./75% RH | | |
|---|---|---|---|---|---|---|---|---|---|
| (073-125 R25-31) | | Methionine | Stopper | | Target | DO₂ | AX-HPLC^{c)} | Methionine | DO₂ |
| Fml ID | Run # | (% w/w) | Type^{a)} | Glass^{b)} | pH | (ppm) | (area-%) | (% w/w) | (ppm) |
| A | 25 | 0 | 1 | 2 | 7.4 | ≦1 | 66.87 | 0 | 0.928 |
| C | 26 | 0.1 | 1 | 2 | 6.5 | ≦1 | 92.41^{d)} | 0.11 | 1.96 |
| D | 27 | 0.25 | 1 | 2 | 6.5 | ≦1 | 91.77 | 0.26 | 1.86 |
| C | 28 | 0.1 | 1 | 2 | 6.5 | ~10 | 84.07 | 0.10 | 8.91 |
| D | 29 | 0.25 | 1 | 2 | 6.5 | ~10 | 78.04 | 0.24 | 8.51 |
| E | 30 | 0.1 | 1 | 2 | 7.4 | ≦1 | 87.32 | 0.09 | 1.84 |
| F | 31 | 0.1 | 1 | 2 | 7.0 | ≦1 | 90.81 | 0.10 | 2.05 |

^{a)}West, Stopper 4432/50 Gray Silicone level 3 Westar RS (uncoated)
^{b)}Untreated, Type I borosilicate glass
^{c)}API control purity is 95.43 area-%, overall purity is out of specification unless otherwise noted
^{d)}Only sum RRT <0.99 ≦3.5, no individual >1.0 is out of specification

TABLE 20
AX-HPLC Purity, % Methionine, and DO2 Measurements of Pegnivacogin
Formulations 040-125, R25-31; 40° C. for 12 Months.

| Formulations | | | | | | Target | 40° C. | | |
|---|---|---|---|---|---|---|---|---|---|
| (073-125 R25-31) | | Methionine | Stopper | | Target | DO₂ | AX-HPLC^{c)} | Methionine | DO₂ |
| Fml ID | Run # | (% w/w) | Type^{a)} | Glass^{b)} | pH | (ppm) | (area-%) | (% w/w) | (ppm) |
| A | 25 | 0 | 1 | 2 | 7.4 | ≦1 | 63.65 | N/A | 1.11 |
| C | 26 | 0.1 | 1 | 2 | 6.5 | ≦1 | 92.36^{d)} | 0.11 | 2.14 |
| D | 27 | 0.25 | 1 | 2 | 6.5 | ≦1 | 92.15^{d)} | 0.26 | 2.22 |
| C | 28 | 0.1 | 1 | 2 | 6.5 | 10 | 82.20 | 0.09 | 9.05 |
| D | 29 | 0.25 | 1 | 2 | 6.5 | 10 | 75.26 | 0.23 | 8.56 |
| E | 30 | 0.1 | 1 | 2 | 7.4 | ≦1 | 88.59 | 0.09 | 2.23 |
| F | 31 | 0.1 | 1 | 2 | 7.0 | ≦1 | 91.56 | 0.10 | 2.19 |

^{a)}West, Stopper 4432/50 Gray Silicone level 3 Westar RS (uncoated)
^{b)}Untreated, Type I borosilicate glass
^{c)}API control purity = 94.38 area-%, overall purity is out of specification unless otherwise noted
^{d)}only sum RRT < 0.99 ≦ 3.5, no individual >1.0 is out of specification

This accelerated data provides significant additional confidence for 3 years stability at 5° C. and that likely 3 years stability at 25° C. or 30° C. is expected to be readily achievable.

### D. AX-HPLC Purity −5° C. Storage Condition up to 24 Months

The AX-HPLC purity values, % methionine, and dissolved oxygen measurements for the samples containing methionine (R26-R31) and the control formulation (R25) stored at 5° C. condition for 24 months are shown in Table 21. Analysis of the samples stored at the 5° C. condition was the only analysis scheduled for these formulations at 24 months. All formulations in this group demonstrated good stability with purity measurements approximately 94-96 area-%, demonstrating virtually identical impurity profiles as compared to the control API (purity is approximately 95 area-%). This data strongly supports the need for an antioxidant in the pegnivacogin formulation to afford the minimum required shelf life of 24 months, but likely 36 months or longer will be achieved. Further, the data clearly demonstrate that even in the presence of ambient DO₂, methionine affords protection of pegnivacogin from degradation at 5° C., thus greatly improving the robustness of the formulation to oxidative stress conditions as compared to the prior formulation.

TABLE 21
AX-HPLC Purity, % Methionine, and DO2 Measurements of Pegnivacogin
Formulations 040-125, R25-31; 5° C. for 24 Months.

| Formulations | | | | | | Target | 5° C. | | |
|---|---|---|---|---|---|---|---|---|---|
| (073-125 R25-31) | | Methionine | Stopper | | Target | DO₂ | AX-HPLCⁱⁱⁱ⁾ | Methionine | DO₂ |
| Fml ID | Run # | (% w/w) | Typeⁱ⁾ | Glassⁱⁱ⁾ | pH | (ppm) | (area-%) | (% w/w) | (ppm) |
| A | 25 | 0 | 1 | 2 | 7.4 | ≦1 | 95.69 | N/A | 0.558 |
| C | 26 | 0.1 | 1 | 2 | 6.5 | ≦1 | 96.39 | 0.11 | 0.455 |
| D | 27 | 0.25 | 1 | 2 | 6.5 | ≦1 | 95.89 | 0.27 | 0.530 |
| C | 28 | 0.1 | 1 | 2 | 6.5 | 10 | 95.72 | 0.11 | 9.57 |
| D | 29 | 0.25 | 1 | 2 | 6.5 | 10 | 96.00 | 0.27 | 9.62 |
| E | 30 | 0.1 | 1 | 2 | 7.4 | ≦1 | 95.55 | 0.10 | 0.510 |
| F | 31 | 0.1 | 1 | 2 | 7.0 | ≦1 | 94.76 | 0.10 | 0.527 |

ⁱ⁾1 = West, Stopper 4432/50 Gray Silicone level 3 Westar RS (uncoated)
ⁱⁱ⁾2 = Untreated, Type I borosilicate glass
ⁱⁱⁱ⁾API control purity = 94.87 area-%

### Example 9

### Ascorbate Formulations

The use of ascorbate as an antioxidant surprisingly had a detrimental effect on purity of the formulation as measured by AX-HPLC. In the absence of ascorbate, purity was measured at 91.78% where in the presence of ascorbate the purity was 72.06%. These results are summarized in Table 22.

TABLE 22
Decreased stability exhibited with known antioxidant (12 months, 5 C.)

| | | RB006_pH 7.4 Na |
|---|---|---|
| Test | RB006_pH 7.4 | Ascorbate |
| Appearance | Clear colorless solution | Clear pale yellow |
| | | solution |
| Purity by AX-HPLC | 91.78% | 72.06% |

Certain modifications and improvements will occur to those skilled in the art upon a reading of the foregoing description. It should be understood that all such modifications and improvements have been deleted herein for the sake of conciseness and readability but are properly within the scope of the general inventive concepts described herein.

1. A pharmaceutical formulation comprising a pegylated aptamer and at least one antioxidant, wherein the formulation has a pH of about 7 or less, an ambient or reduced level of dissolved oxygen and a shelf life of at least about 24 months or at least about 36 months.

2. The pharmaceutical formulation of claim 1, wherein the formulation has an ambient level of dissolved oxygen.

3. The pharmaceutical formulation of claim 1, wherein the level of dissolved oxygen is from about 5 to about 10 ppm.

4. The pharmaceutical formulation of claim 1, wherein the formulation has a reduced level of dissolved oxygen.

5. The pharmaceutical formulation of claim 1, wherein the level of dissolved oxygen is less than about 5 ppm.

6. The pharmaceutical formulation of claim 1, wherein the concentration of the pegylated aptamer is from about 1 to about 100 mg/mL.

7. The pharmaceutical formulation of claim 1, wherein the concentration of the pegylated aptamer is from about 20 to about 30 mg/mL.

8. The pharmaceutical formulation of claim 1, wherein the concentration of the pegylated aptamer is from 24 mg/mL.

9. The pharmaceutical formulation of claim 1, wherein methionine is present from about 0.001 to about 0.50% w/w, about 0.10 to about 0.25 or about 0.10% w/w.

10. The pharmaceutical formulation of claim 9, wherein, methionine is present at about 0.10% w/w.

11. The pharmaceutical formulation of claim 1, wherein the pegylated aptamer is pegnivacogin.

12. The pharmaceutical formulation of claim 1, wherein the pegylated apatamer is RB571.

13. The pharmaceutical formulation of claim 1, wherein shelf life is determined at about 2° C. to about 8° C.

14. The pharmaceutical formulation of claim 1, wherein shelf life is determined at about 25° C. to about 30° C.

15. A pharmaceutical formulation comprising about 24 mg/mL of pegnivacogin and about 0.1% methionine, wherein the formulation has a pH of about 6.8, an ambient or reduced level of dissolved oxygen and a shelf life of at least about 24 months or at least about thirty six (36) months.

16. The pharmaceutical formulation of claim 15, wherein the level of dissolved oxygen is ambient.

17. The pharmaceutical formulation of claim 15, wherein the level of dissolved oxygen is between about 5 and about 10 ppm.

18. The pharmaceutical formulation of claim 15, wherein the level of dissolved oxygen is less than about 5 ppm.

19. The pharmaceutical formulation of claim 15, wherein shelf life is determined at about 2 to about 8° C.

20. The pharmaceutical formulation of claim 15, wherein the shelf life is determined at about 25 to about 30° C.

21. A method of preparing a pharmaceutical formulation, comprising: (i) providing an aqueous solvent comprising methionine; (b) adding a pegylated aptamer to the aqueous solvent to form an aqueous formulation; (c) filtering the aqueous formulation to form a filtered aqueous formulation; (d) filling a storage container with the filtered aqueous formulation; and (e) sealing the storage container to provide a pharmaceutical formulation having a pH of about 7 or less, an ambient or reduced level of dissolved oxygen and a shelf life of at least about 24 months or at least about thirty six (36) months.

22. The method of claim 21, further comprising reducing the pH one or more times prior to (b), (c), (d) or (e).

23. The method of claim 21, wherein the pharmaceutical formulation has a reduced level of dissolved oxygen.

24. The method of claim 23, wherein the level of dissolved oxygen is reduced one or more times prior to (a), (b), (c), (d) or (e).

25-29. (canceled)

30. The method of claim 21, wherein the pegylated aptamer is pegnivacogin.

31. The method of claim 21, wherein the pegylated aptamer is RB571.

32. The method of claim 21, wherein shelf life is determined at about 2° C. to about 8° C.

33. The methods of claim 21, wherein the shelf life is determined at about 25 to about 30° C.

34-51. (canceled)

52. A method of treating a host in need thereof by administering a therapeutically effective amount of the pharmaceutical formulation of claim 1.

53-72. (canceled)
